(19) United States
(12) Patent Application Publication
(10) Pub. No.: US 2024/0279315 A1
(43) Pub. Date: Aug. 22, 2024

(54) UNGULATE-DERIVED POLYCLONAL IMMUNOGLOBULIN SPECIFIC FOR INFLUENZA VIRUS AND USES THEREOF

(71) Applicant: SAB, LLC, Sioux Falls, SD (US)

(72) Inventors: Eddie J. SULLIVAN, Sioux Falls, SD (US); Hua WU, Sioux Falls, SD (US); Christoph L. BAUSCH, Sioux Falls, SD (US); Thomas LUKE, Sioux Falls, SD (US)

(73) Assignee: SAB, LLC, Sioux Falls, SD (US)

(21) Appl. No.: 18/444,239

(22) Filed: Feb. 16, 2024

Related U.S. Application Data
(60) Provisional application No. 63/485,688, filed on Feb. 17, 2023.
(60) Provisional application No. 63/505,580, filed on Jun. 1, 2023.

(51) Int. Cl. C07K 16/10; A61K 39/00; A61P 31/16; C07K 1/107

(52) U.S. Cl. CPC C07K 16/1018; A61P 31/16; C07K 1/1077; A61K 39/00; A61K 2039/505; C07K 2317/21; C07K 2317/76

(57) ABSTRACT

Provided are human polyclonal immunoglobulin products specific for influenza hemagglutinin (HA) protein for use in treating or preventing disease associated with an influenza infection. Further provided herein are methods for making such human polyclonal immunoglobulins in a transgenic ungulate, such as, but not limited to, using a transchromosomic bovine (TcB) system.

### CROSS-REFERENCE TO RELATED APPLICATIONS

This application claims priority to and benefit of U.S. Provisional Application No. 63/485,688, filed Feb. 17, 2023, and U.S. Provisional Application No. 63/505,580, filed Jun. 1, 2023, the disclosures of each of which are hereby incorporated by reference in their entirety.

### TECHNICAL FIELD

The invention relates to ungulate-derived polyclonal human immunoglobulin compositions for treatment of disease associated with influenza virus.

### BACKGROUND

Influenza causes substantial morbidity and mortality worldwide despite available antivirals and vaccines. Influenza is responsible for 226,000 excess hospitalizations and 30,000 to 50,000 deaths each year in the United States alone. Effective therapeutics are needed to prevent mortality or morbidity in those afflicted with severe influenza. Human plasma (delivered as Fresh Frozen Plasma units) or human intravenous immunoglobulin (hIVIg) with anti-influenza antibodies have been proposed as treatments for severe influenza.

A limitation of human convalescent influenza plasma or anti-Flu hIVIg (prepared from large numbers of human plasma units) is that they must be screened to identify those few with a higher-than-average hemagglutination inhibition (HAI) titers to two or more strains of influenza. Additionally, recent clinical trials have not shown a benefit to hospitalized patients with severe Type A influenza infections treated with human-derived anti-influenza plasma or anti-Flu hIVIg.

Influenza is also known to mutate rapidly with small drifts over time and occasional shifts resulting in novel strains arising frequently to infect humans.

There exists a need for immunoglobulin compositions for therapeutic use in patients suffering from or at risk for influenza infection that can keep up with the ever-changing influenza virus.

### SUMMARY

The present inventors have developed an ungulate-derived polyclonal human immunoglobulin composition for the treatment of influenza virus associated disease. The composition is made from purified pooled plasma from Transchromosomic (Tc) bovines genetically engineered to produce polyclonal human antibodies having human polypeptide sequences that have been immunized with two or more strains of influenza. An anti-influenza hemagglutinin (HA) protein human polyclonal immunoglobulin composition can have substantial therapeutic and safety benefits compared to monoclonal antibody therapy. The pooling strategy described herein, where the plasma from transchromosomic bovines immunized with two or more seasonal influenza strains are combined, provides a therapeutic immunoglobulin composition to effectively treat the ever-changing influenza virus.

In one aspect, the disclosure provides an ungulate-derived polyclonal human immunoglobulin composition, comprising a population of polyclonal human immunoglobulins, wherein the population of polyclonal human immunoglobulins binds an influenza hemagglutinin (HA) protein from two or more strains of influenza. In some embodiments, the influenza HA protein from two or more strains of influenza can be HA protein from Influenza A and/or HA protein from Influenza B.

In some embodiments, the composition is produced by immunizing a transgenic ungulate with an effective amount of an influenza HA protein. In some embodiments, the influenza HA protein is from two or more strains of influenza. The amount of influenza HA protein used for immunization can be from about 0.1 to 10 mg of each influenza HA protein.

In some embodiments, the influenza HA protein of the two or more strains of influenza comprises a full-length HA1 protein and/or a full-length HA2 protein.

In some embodiments, the population of polyclonal human immunoglobulins can block influenza HA protein from binding to sialic acid. In some embodiments, the population of polyclonal immunoglobulins has an HAI titer of at least 64 or 512.

In some embodiments, the population of polyclonal human immunoglobulins blocks Influenza A virus and/or Influenza B virus from infecting a mammalian cell.

In some embodiments, the population of polyclonal human immunoglobulins increases survival after Influenza A and/or Influenza B infection.

In some embodiments, the population of polyclonal human immunoglobulin prevents or decreases lower and/or upper respiratory symptoms after Influenza A and/or Influenza B infection.

In some embodiments, the population of polyclonal human immunoglobulin prevents or decreases fever, malaise, or fatigue.

In some embodiments, the population of polyclonal human immunoglobulin decreases sneezing after Influenza A and/or Influenza B infection.

In some embodiments, the population of polyclonal human immunoglobulins decreases viral titer in vivo.

In some embodiments, the population of polyclonal human immunoglobulins has a neutralizing concentration of at least 0.01 μg/ml, at least 0.1 μg/ml, or at least 1.0 μg/ml.

In some embodiments, the population of polyclonal human immunoglobulins has a neutralizing concentration of 0.01 μg/ml to 0.1 μg/ml, or 0.1 μg/ml to 1.0 μg/ml.

In some embodiments, the population of polyclonal human immunoglobulins has an avidity for influenza HA protein of at least 0.1 1/sec, at least 0.01 1/sec, at least 0.001 1/sec at least 0.0001 1/sec, or at least 0.00001 1/sec.

In some embodiments, the population of polyclonal human immunoglobulins has an avidity for influenza HA protein of 0.1 to 0.01×1/sec, 0.01 to 0.001×1/sec, 0.001 to 0.0001×1/sec, or 0.0001 to 0.00001×1/sec.

In some embodiments, the population of human immunoglobulins has an avidity for influenza HA protein of at two or more strains of influenza.

In some embodiments, the population of polyclonal human immunoglobulins comprise glycans covalently linked to the human immunoglobulins. The glycans can comprise at least about 70% N-Glycolylneuraminic acid (NGNA) glycans, for example about 90% N-Glycolylneuraminic acid (NGNA) glycans. In some embodiments, the glycans can comprise at least about 5% N-acetylneuraminic acid (NANA)-bearing glycans e.g., at least about 10% NANA bearing glycans.

In some embodiments, the glycan can comprise less than about 50% NANA glycans, e.g., less than about 20% NANA glycans.

In some embodiments, the population of polyclonal human immunoglobulins can comprise less than 5% chimeric IgG and/or IgM immunoglobulins. In some embodiments, the population of human immunoglobulins comprises at least about 70% of IgG1.

In some embodiments, the population of human immunoglobulins can comprise at least about 70% IgG1 e.g., about 90% IgG1. In some embodiments, the population of human immunoglobulins can comprise less than about 30% IgG2 e.g., about 10% IgG2. In some embodiments, the population of immunoglobulins can comprise less than about 4% of one or more of IgG3 and IgG4.

In some embodiments, the disclosure provides a method of making polyclonal human immunoglobulin specific for hemagglutinin (HA), comprising administering an effective amount of an influenza HA from at least two or more strains of influenza, or a polynucleotide encoding an influenza HA protein from at least two or more strains of influenza, to one or more transgenic ungulates, wherein the one or more transgenic ungulates comprise a genome comprising a human immunoglobulin locus or an artificial chromosome comprising a human immunoglobulin locus.

In some embodiments, the method involves combining the ungulate-derived polyclonal immunoglobulins purified from one or more transgenic ungulates. In some embodiments, the one or more transgenic ungulates produce a population of human immunoglobulins that specifically binds HA.

In some embodiments, the method comprises administering the influenza HA protein or polynucleotide encoding the influenza HA protein 3, 4, 5, or more times. In some embodiments, the influenza HA protein is administered via an intramuscular route, an intranasal route, a subcutaneous route, or an oral route.

In some embodiments, the method comprises collecting serum or plasma from the transgenic ungulate. In some embodiments, the method involves combining the ungulate-derived polyclonal immunoglobulins purified from one or more transgenic ungulates. In some embodiments, the method utilizes HA proteins from two or more strains of influenza.

In some embodiments, the serum or plasma comprises a population of fully human immunoglobulins.

In some embodiments, the antigenic fragment of influenza HA protein is an influenza HA extracellular domain.

In some embodiments, the population of human immunoglobulins blocks influenza HA protein from binding to sialic acid.

In some embodiments, the population of human immunoglobulins blocks Influenza A virus and/or Influenza B virus from infecting a mammalian cell.

In some embodiments, the population of human immunoglobulin increases survival after Influenza A and/or Influenza B infection.

In some embodiments, the population of human immunoglobulin decreases sneezing after Influenza A and/or Influenza B infection.

In some embodiments, the population of human immunoglobulins decreases viral titer in vivo.

In some embodiments, the population of human immunoglobulins has a neutralizing concentration of at least 0.01 μg/ml, at least 0.1 μg/ml, or at least 1.0 μg/ml.

In some embodiments, the population of human immunoglobulins has a neutralizing concentration of 0.01 μg/ml to 0.1 μg/ml, or 0.1 μg/ml to 1.0 μg/ml.

In some embodiments, the population of human immunoglobulins has an avidity for influenza HA protein of at least 0.1×1/sec, at least 0.01×1/sec, at least 0.001×1/sec at least 0.0001×1/sec, or at least 0.00001×1/sec.

In some embodiments, the population of human immunoglobulins has an avidity for influenza HA protein of 0.1 to 0.01×1/sec, 0.01 to 0.001×1/sec, 0.001 to 0.0001×1/sec, or 0.0001 to 0.00001×1/sec.

In some embodiments, the population of human immunoglobulins has an avidity for influenza HA protein for two or more strains of influenza, wherein the two or more strains are from the same or different years or seasons.

In some embodiments, the method comprises: a) administering a polynucleotide encoding an antigenic fragment of HA from two or more strains of influenza; b) administering a polynucleotide encoding the antigenic fragment of HA from two or more strains of influenza, three to four weeks later; c) administering the antigenic fragment of HA from two or more strains of influenza, four weeks later d) administering the antigenic fragment of HA from two or more strains of influenza, four weeks later; and e) administering the antigenic fragment of HA from two or more strains of influenza, four weeks later. In some embodiments, the influenza HA protein can be administered with one or more excipients. The excipients can be sodium chloride, monobasic sodium phosphate, dibasic sodium phosphate and/or polysorbate 20 (Tween®20).

In some embodiments, the method comprises purifying the human immunoglobulin to produce a composition.

Also provided herein are human immunoglobulins prepared by the methods described herein.

In some embodiments, the method comprises a pharmaceutical composition, comprising the composition and optionally one or more pharmaceutically acceptable excipients.

A method of treating disease associated with influenza virus in a subject in need thereof, comprising administering an effective amount of the composition or a pharmaceutical composition to the subject.

Additional embodiments, features, and advantages of the invention will be apparent from the following detailed description and through practice of the invention.

### BRIEF DESCRIPTION OF THE DRAWINGS

FIGS. 1A-1H show construction of the HAC vector and KcHACΔ vector.

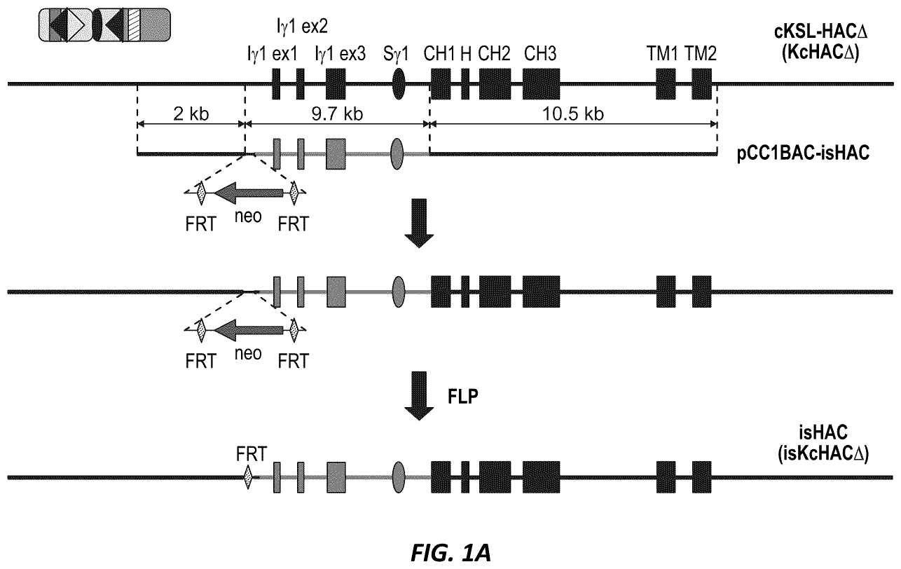

FIG. 1A shows a flow of the isHAC and isKcHACΔ vector construction. The bovinizing vector, pCC1BAC-isHAC, is BAC-based (backbone is pCC1BAC vector), comprising 10.5 kb and 2 kb of genomic DNA as a long and short arm, respectively, 9.7 kb of the bovine genomic DNA covering the bovine Iγ1-Sγ1 and its surrounding region to replace the human corresponding 6.8 kb of I71-Sγ1 region, the chicken β-actin promoter-driven neo gene flanked by FRT sequence and DT-A gene. After the targeted bovinization, the neo cassette is removed by FLP introduction.

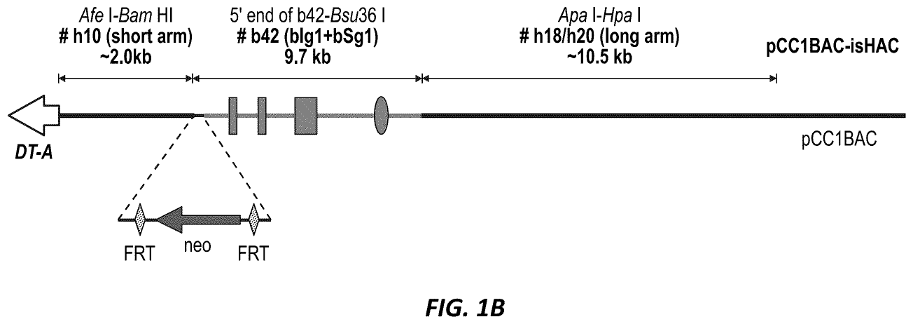

FIG. 1B shows detailed information on the targeting vector pCC1BAC-isHAC. The 2 kb of Afe I-Bam HI fragment and 10.5 kb of Apa I-Hpa I fragment for a short arm and long arm were obtained from clone h10 and clone h18/h20, respectively, derived from λ, phage genomic library constructed from CHO cells containing the κHAC vector by screening using a probe around the human Iγ1-Sγ1 region. The 9.7 kb fragment (5′ end through Bsu36 I) was obtained from clone b42 derived from the λ phage bovine genomic library.

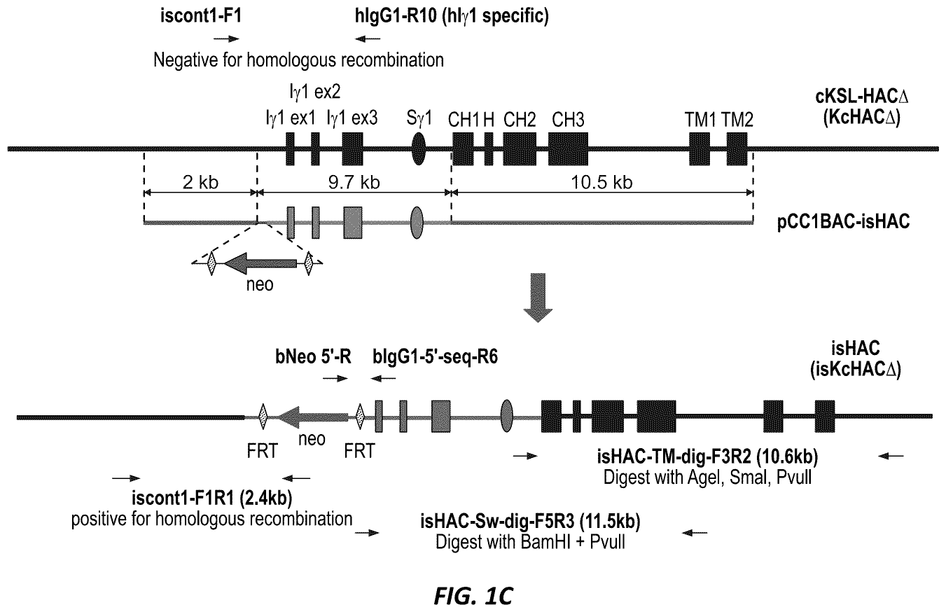

FIG. 1C shows genotyping of the bovinized Iγ1-Sγ1 region. Five sets of PCR primers that amplify genomic PCR were implemented, as indicated. The iscont1-F1/R1 primer set is a positive PCR specific to the homologous recombination. The iscont1-F1×hIgG1-R10 is a negative PCR that is prohibited by the presence of the neo cassette. isHAC-Sw-dig-F5/R3 and isHAC-TM-dig-F3/R2 are for structural integrity check of their corresponding region, digested by Bam HI+Pvu II and Age I, Sma I or Pvu II, respectively. The primer set, bNeo 5′-R×bIgG1-5′-seq-R6, is used to confirm the presence of FRT sequence.

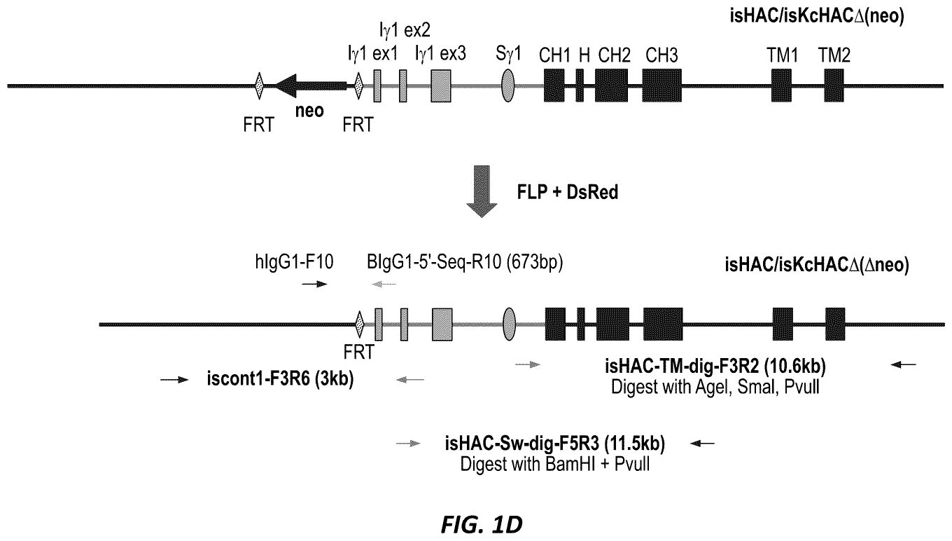

FIG. 1D shows genotyping after the FLP-FRT deletion of the neo cassette.

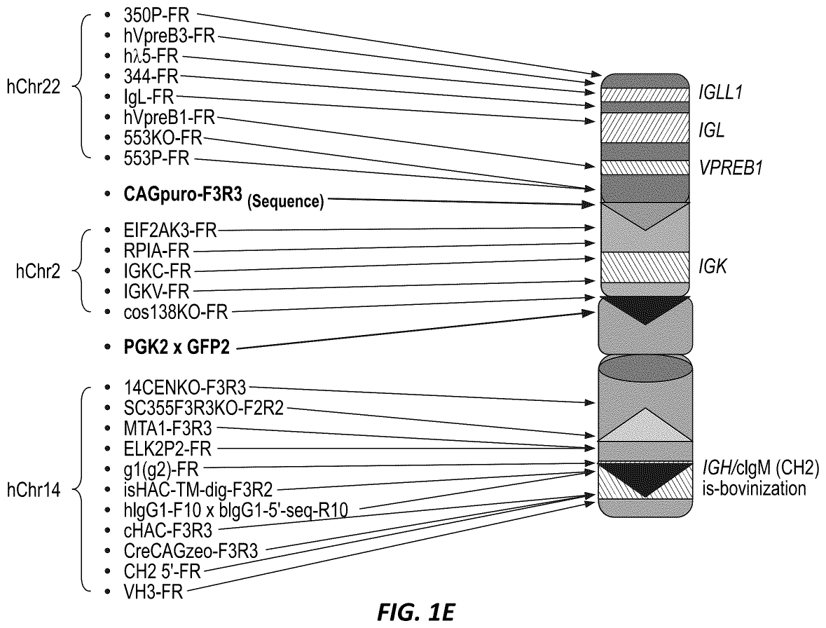

FIG. 1E shows extensive genomic PCR for genotyping of the isHAC vector. Location of each genomic PCR primer pair is depicted in relation to the isHAC vector structure.

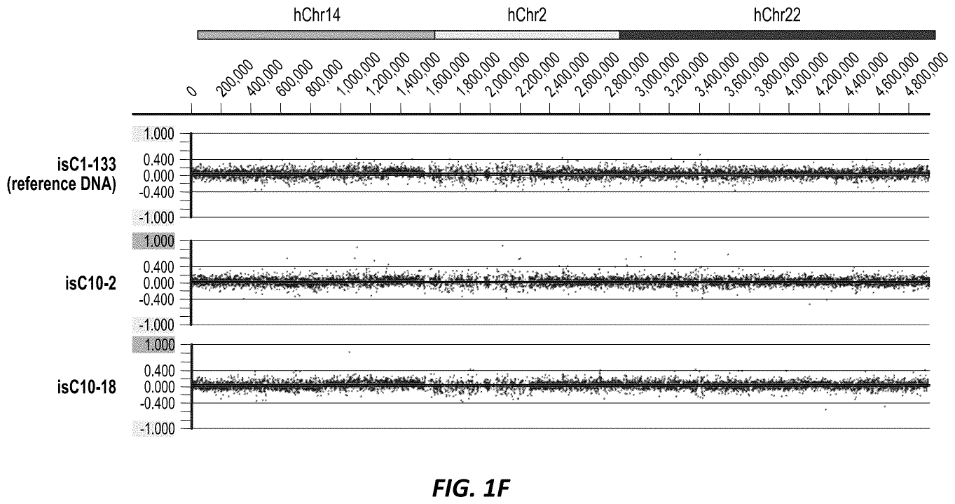

FIG. 1F shows CGH analysis among three different CHO clones containing the isHAC vector. DNA from isC1-133 was used as a reference. There was no apparent structural difference of the isHAC vector among the three cell lines.

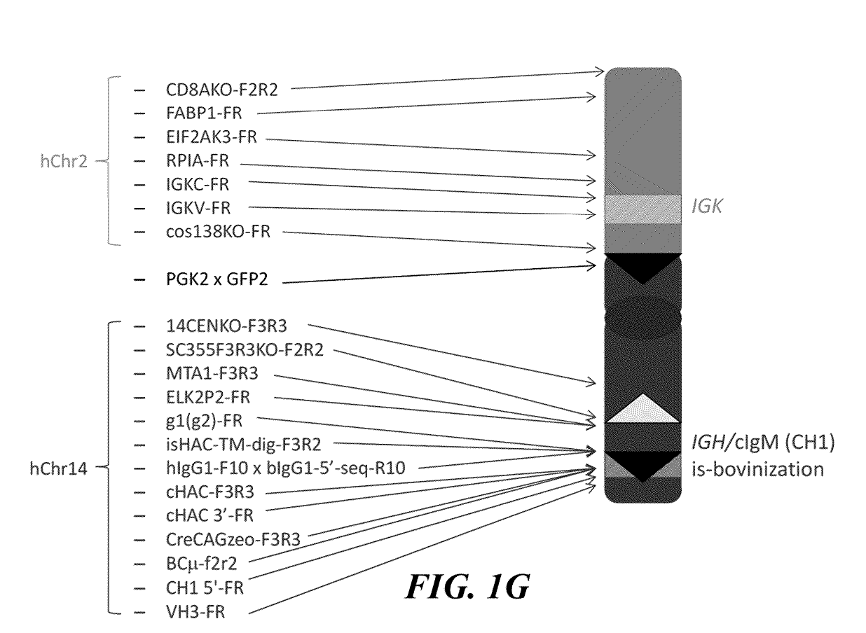

FIG. 1G shows extensive genomic PCR for genotyping of the isKcHACΔ vector. Location of each genomic PCR primer pair is depicted in relation to the isKcHACΔ vector structure.

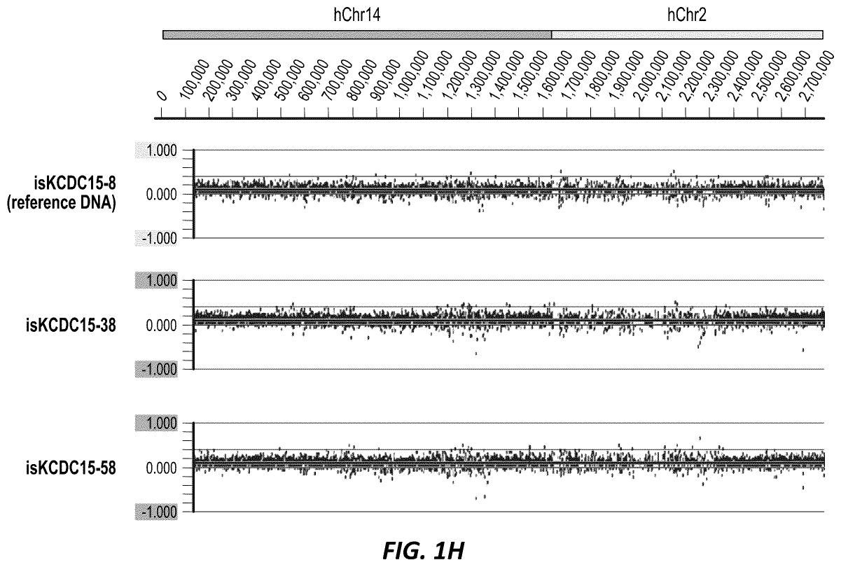

FIG. 1H shows CGH analysis among three different CHO clones containing the isKcHACΔ vector. DNA from isKCDC15-8 was used as a reference. There was no apparent structural difference of the isKcHACΔ among the three cell lines.

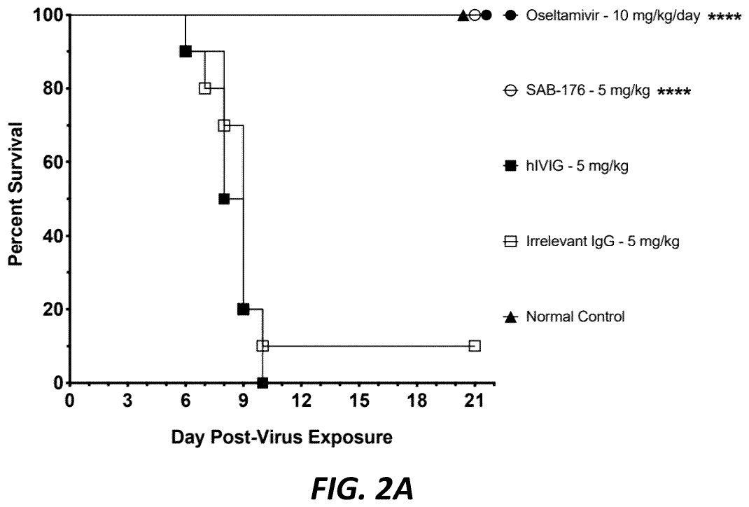

FIG. 2A shows survival and FIG. 2B shows mean body weights of mice following intraperitoneal treatment with SAB-176 (ungulate-derived human polyclonal immunoglobulin composition specific for influenza virus) or anti-Flu hIVIg (a human-derived anti-influenza immunoglobulin) after challenge with A/CA/04/2009 (H1N1pdm) influenza virus. A) Intraperitoneal administration of SAB-176 provided complete protection from mortality while intraperitoneal anti-Flu hIVIg did not provide any protection from mortality. B) Neither SAB-176 or anti-Flu hIVIg treatments given intraperitoneally were able to provide protection from weight loss. (*P<0.05, ****P<0.0001 compared to mice treated with irrelevant IgG.)

FIG. 3 shows survival of mice following intraperitoneal treatment with SAB-176, anti-Flu hIVIg, or irrelevant IgG after challenge with A/HK/2369/09 H275Y (H1N1pdm) influenza virus (n=10 mice/group). Mice treated with a single administration of SAB-176 at a dose of 5, 10, or 20 mg/kg completely protected mice from mortality. Three of ten mice treated with anti-Flu hIVIg at a dose of 5 mg/kg survived the infection. Two of ten mice treated with anti-Flu hIVIg at a dose of 10 mg/kg survived the infection. None of the ten mice treated with anti-Flu hIVIg at a dose of 20 mg/kg survived the infection. None of the placebo-treated mice survived the infection. (****P<0.0001 compared to placebo-treated mice.)

FIG. 4 shows mean body weights of mice following intraperitoneal treatment with SAB-176, anti-Flu hIVIg, or irrelevant IgG after challenge with A/HK/2369/09 H275Y (H1N1pdm) influenza virus (n=10 mice/group). Treatment with oseltamivir at a dose of 10 mg/kg/d did not protect mice from weight loss. A single IP administration of SAB-176 at a dose of 5, 10, or 20 mg/kg protected mice from weight loss. A single IP administration of anti-Flu hIVIg at a dose of 5, 10, or 20 mg/kg did not protect mice from weight loss (****P<0.0001 compared to placebo-treated mice.)

FIG. 5 shows ferret weights over time after challenge. Ferrets were infected with pH1N1 influenza virus and weighed from day −1 through day 4 post-challenge. Weights are presented as a percent of the initial body weight, which was collected on Day −1. Results for the SAB-176 group are reported as the mean±standard deviation (n=3). Results for the Control IgG are presented for the individual ferret in this group.

FIG. 6 shows ferret body temperature over time after challenge. Rectal temperatures were taken daily from day −1 through day 4. Temperatures are reported in degrees Celsius. Results for the SAB-176 group are reported as the mean±standard deviation (n=3). Results for the Control IgG are presented for the individual ferret in this group.

FIG. 7 shows ferret clinical scores over time after challenge. Ferrets were monitored daily for 10 minutes, and a clinical score was calculated based on their activity and sneezing during the observation period. Results for the SAB-176 group are reported as the mean±standard deviation (n=3). Results for the Control IgG are presented for the individual ferrets in this group.

FIG. 8 shows ferret sneezes over time after challenge. Ferrets were monitored daily for 10 minutes, and sneezes were recorded during the observation period. Results for the SAB-176 group are reported as the mean±standard deviation (n=3). Results for the Control IgG are presented for the individual ferret in this group.

FIG. 9 shows nasal wash virus titers. Nasal washes were collected from ferrets on days −1, 1, 2, 3, and 4 of the study. MDCK cells were inoculated with serial (1:10) dilutions of nasal wash samples, and 50% tissue culture infectious dose titers were calculated using the method of Reed and Muench (15). Titers are reported for individual animals, with the line representing the mean titer for the group that received SAB-176.

FIG. 10 shows lung virus titers at day 4 post challenge. Lungs were collected from ferrets on day 4 of the study. MDCK cells were inoculated with serial (1:10) dilutions of lung homogenate samples, and 50% tissue culture infectious dose titers were calculated using the method of Reed and Muench (15). Titers are reported for individual animals, with the line representing the mean titer for the group that received SAB-176. Lungs were collected from ferrets on day 4 of the study.

FIG. 11 shows olfactory bulb virus titers. Olfactory bulbs were collected from ferrets on day 4 of the study. MDCK cells were inoculated with serial (1:10) dilutions of olfactory bulb homogenate samples, and 50% tissue culture infectious dose titers were calculated using the method of Reed and Muench (15). Titers are reported for individual animals, with the line representing the mean titer for the group that received SAB-176.

FIG. 12 shows soft palate virus titers. Soft palates were collected from ferrets on day 4 of the study. MDCK cells were inoculated with serial (1:10) dilutions of soft palate homogenate samples, and 50% tissue culture infectious dose titers were calculated using the method of Reed and Muench (15). Titers are reported for individual animals, with the line representing the mean titer for the group that received SAB-176.

FIG. 13 shows HAI titers over time. Blood was collected from ferrets on days −1, 2, 3, and 4 of the study. Serum was separated from the blood samples, treated with Receptor Destroying Enzyme (RDE), and tested for hemagglutinin inhibition activity against the A/California/7/09 (H1N1), A/Singapore/INFIMH-16-0019/2016 (H3N2), B/Phuket/3073/2013-BYam, or B/Maryland/15/2016-BVic influenza viruses. Serum samples were diluted starting at 1:10 and results are shown for individual serum samples.

FIG. 14 shows the mean total viral load by nasal samples by qRT-qPCR by day relative to viral challenge. The area under the curve shows qRT-PCR results from nasopharyngeal swabs taken through 8 days after study participants were intranasally inoculated with infectious A/California/2009 H1N1 virus (per protocol analysis set). The solid line indicates participants randomized to 25 mg/kg of SAB-176 (n=29). The dashed line indicates participants randomized to an equal volume of normal saline (n=30). Values listed are mean (+/−1 SE) viral load (log 10 copies/ml) from both groups and differences were statistically significant (P-value of One-sided Wilcoxon rank sum test (0.026)).

FIG. 15 shows the mean total symptom score by day relative to viral challenge. The area under the curve for 13-point diary card symptom shows scores taken through 8 days after study participants were intranasally inoculated with infectious A/California/2009 H1N1 virus (per protocol analysis set). The solid line indicates participants randomized to 25 mg/kg of SAB-176 (n=29). The dashed line indicates participants randomized to an equal volume of normal saline (n=30). Values shown are mean (+/−1 SE) total symptoms from both groups and differences were almost statistically significant (P-value of One-sided Wilcoxon rank sum test (0.066)).

FIG. 16 shows the Kaplan-Meier time to resolution of positive viral cultures after intranasal viral challenge.

FIG. 17 is a line graph showing human IgG (mg/ml) levels in goat kids.

FIG. 18 is a line graph showing survival of mice following treatment with transgenic goat-derived human IgG, 12 hours post-virus challenge with influenza A/Anhui/3/2013. The 25 and 100 mg/kg doses of the goat-derived antibody provided 70 and 80% protection, respectively. The 50 mg/kg dose of SAB-127, used as a positive control provided 70% protection. (*P<0.05, **P<0.01).

FIG. 19 is a line graph showing mean body weights following treatment with transgenic goat-derived human IgG, 12 hours post-virus challenge with influenza A/Anhui/3/2013. None of the groups treated with the goat-derived antibody showed protection from weight loss (*P<0.05).

### DETAILED DESCRIPTION

Provided herein are ungulate-derived polyclonal human immunoglobulin compositions for treatment of influenza in humans that overcomes limitations of monoclonal antibody therapy. Transgenic animals with the endogenous immunoglobulin (Ig) locus replaced by a human artificial chromosome encoding a human Ig locus express fully human polyclonal antibodies. Immunization of such a transgenic animal with two or more recombinant influenza HA proteins, or an antigenic fragment thereof, and/or with a polynucleotide encoding the two or more antigens, generates ungulate-derived polyclonal human immunoglobulin compositions with yield, purity, and antigen specificity that enable use of this composition in medical applications. The present disclosure provides ungulate-derived polyclonal human immunoglobulin compositions that are prepared by immunizing one or more groups of transgenic ungulates with seasonal or yearly variants of influenza and subsequently pooling the resultant plasma obtained from the groups of transgenic ungulates.

### Definitions

All embodiments of any aspect of the invention can be used in combination, unless the context clearly dictates otherwise.

Words using the singular or plural number also include the plural and singular number, respectively. Additionally, the words “herein,” “above,” “below,” and words of similar import, when used in this application, shall refer to this application as a whole and not to any particular portions of the application.

The term “ungulate” refers to any suitable ungulate, including but not limited to bovine, pig, horse, donkey, zebra, deer, oxen, goats, sheep, and antelope.

The term “transgenic” means the cells of the ungulate comprise one or more polynucleotides encoding exogenous gene(s) (e.g., an immunoglobulin locus). Such as polynucleotide can be a portion of an artificial chromosome. Alternatively, or in addition to an artificial chromosome, one or more polynucleotides encoding exogenous gene(s) can be integrated into the genome of the cells of the ungulate.

The term “influenza hemagglutinin protein,” “influenza HA protein,” “HA protein,” or “HA” as used herein refers to a glycoprotein found on the surface of influenza viruses that is responsible for binding of the virus to the cell that is being infected. The influenza hemagglutinin protein can bind to monosaccharide sialic acid, which can be present on the surface of its target or host cell. When the host cell is a red blood cell (erythrocyte), the influenza hemagglutinin protein can cause red blood cells (erythrocytes) to clump together (“agglutinate”) in vitro. Influenza HA proteins can include influenza HA0 protein, influenza HA1 protein and/or influenza HA2 protein. The influenza hemagglutinin protein is organized as a noncovalently associated homotrimer on the viral surface. Each monomer of HA is post translationally cleaved into HA1 and HA2 proteins that are linked by disulfide bonds. In some embodiments, the precursor form of influenza HA protein where the HA1 and HA2 proteins are not post translationally cleaved can be referred to as HA0 protein.

In an embodiment, HA antigens include full length proteins containing the transmembrane domain and the HA1 and HA2 regions. Any influenza HA antigen or combination of HA influenza proteins (e.g., 1, 2, 3, 4, 5, 6, 7, 8, 9, 10 or more HA influenza proteins) can be used in the methods described herein. Recombinant or non-recombinant HA proteins can be used.

Recombinant HA proteins can form trimeric structures and can be cleaved or uncleaved, Recombinant HA proteins can be produced in, for example, insect cells, eggs, or egg cells and purified using, for example, a combination of filtration and column chromatography methods. Any suitable method can be used to obtain recombinant HA proteins. In an example, any suitable influenza vaccine strain (e.g., H1N1, H3N2, Influenza B (e.g., B Victoria lineage, Yamagata lineage)) can be obtained. In one aspect, two or more influenza vaccine strains are obtained. The full-length HA gene (containing the HA1 and HA2 genes) can be cloned using RT-PCR and inserted into a baculovirus transfer vector, containing, for example, the promoter from the baculovirus polyhedrin gene flanked by sequences naturally surrounding the polyhedrin locus. The transfer vector can be co-transfected into insect cells with the linearized baculovirus genomic DNA (e.g., Autographa Californica Nuclear Polyhedrosis Virus) depleted of the polyhedrin gene and part of an essential gene downstream of the polyhedrin locus. Homologous recombination can occur between the transfer plasmid and the linearized viral DNA thereby rescuing the virus, resulting in recombinant viruses. Recombinant viruses can be selected by, e.g., plaque assay. Plaque-derived recombinant baculovirus can then be used to make a virus stock by infecting insect cells in serum-free culture medium. The virus stock can then be used to infect insect cells to produce recombinant HA.

The terms “polyclonal” or “polyclonal serum” or “polyclonal plasma” or “polyclonal immunoglobulin” refer to a population of immunoglobulins having shared constant regions but diverse variable regions. The term polyclonal does not, however, exclude immunoglobulins derived from a single B cell precursor or single recombination event, as may be the case when a dominant immune response is generated. A polyclonal serum or plasma contains soluble forms (e.g., IgG) of the population of immunoglobulins. The term “purified polyclonal immunoglobulin” refers to polyclonal immunoglobulin purified from serum or plasma. Methods of purifying polyclonal immunoglobulin include, without limitation, caprylic acid fractionation and adsorption with red blood cells (RBCs).

A “population” of immunoglobulins refers to immunoglobulins having diverse sequences, as opposed to a sample having multiple copies of a single immunoglobulin. Similarly stated, the term “population” excludes immunoglobulins secreted from a single B cell, plasma cell, or hybridoma in culture, or from a host cells transduced or transformed with recombinant polynucleotide(s) encoding a single pair of heavy and light chain immunoglobulin sequences.

The term “immunoglobulin” refers to a protein complex of at least two heavy and at least two light chains in 1:1 ratio, including any of the five classes of immunoglobulin—IgM, IgG, IgA, IgD, IgE. In variations, the immunoglobulin is engineered in any of various ways known in the art or prospectively discovered, including, without limitation, mutations to change glycosylation patterns and/or to increase or decrease complement dependent cytotoxicity.

An immunoglobulin is “fully human or substantially human” when the protein sequence of the immunoglobulin is sufficiently similar to the sequence of a native human immunoglobulin that, when administered to a subject, the immunoglobulin generates an anti-immunoglobulin immune response similar to, or not significantly worse, that the immune reaction to native human immunoglobulin. A fully human immunoglobulin will comprise one or more substitutions, insertions, to deletions in variable regions, consistent with recombination, selection, and affinity maturation of the immunoglobulin sequence. In variations, the fully human or substantially human immunoglobulin can be engineered in any of various ways known in the art or prospectively discovered, including, without limitation, mutations to change glycosylation patterns and/or to increase or decrease complement dependent cytotoxicity.

The term “isolated” means that the material is removed from its original environment (e.g., the natural environment if it is naturally occurring). For example, a naturally occurring nucleic acid molecule or polypeptide present in a living animal is not isolated, but the same nucleic acid molecule or polypeptide, separated from some or all of the co-existing materials in the natural system, is isolated. Such a nucleic acid molecule could be part of a vector and/or such a nucleic acid molecule or polypeptide could be part of a composition (e.g., a cell lysate), and still be isolated in that such a vector or composition is not part of the natural environment for the nucleic acid molecule or polypeptide. Any of the compositions of the present disclosure can be isolated compositions.

The percentage of an immunoglobulin (e.g., immunoglobulin that binds HA) “by mass of total immunoglobulin” refers to the concentration of a target immunoglobulin population divided by the concentration of total immunoglobulin in a sample, multiplied by 100. The concentration of target immunoglobulin can be determined by, for example, affinity purification of target immunoglobulin (e.g., on affinity column comprising HA) followed by concentration determination.

The term “about” or “approximately” means plus or minus a range of up to 5%.

The terms “immunization” and “immunizing” refer to administering a composition to a subject (e.g., a transgenic ungulate) in an amount sufficient to elicit, after one or more administering steps, a desired immune response (e.g., a polyclonal immunoglobulin response specific to HA). Administration can be by intramuscular injection, intravenous injection, intraperitoneal injection, or any other suitable route. Immunization can comprise between one and ten, or more administrations (e.g., injections) of the composition, such as 1, 2, 3, 4, 5, 6, 7, 8, 9, 10, or more administrations. The first administration can elicit no detectable immune response as generally each subsequence administration will boost the immune response generated by prior administrations.

The term “target antigen” refers to any antigen use to elicit a desired immune response. The target antigen used to generate an immunoglobulin composition can be recombinant influenza HA protein or an antigenic fragment thereof, or nucleic acid molecule that encodes such proteins (e.g., RNA, m RNA, linear DNA, or plasmid DNA).

The term “variant” as used herein, refers to a viral genome that may contain one or more mutations relative to a reference viral genome. A variant may be a Variant of Interest (VOI), Variant of Concern (VOC), Variant of High Consequence (VOHC) or Variant Being Monitored (VBM) due to shared attributes and characteristics that may require public health action. A “seasonal variant” can be a variant that occurs at a certain period of time of a particular year. For influenza, the period of time for the Northern Hemisphere may be between about September to March. In some embodiments, a seasonal variant is also called a yearly variant.

The term “purify” refers to separating a target cell or molecule (e.g., a population of immunoglobulins) from other substances present in a composition. Immunoglobulins can be purified by fractionation of plasma, by affinity (e.g., protein A or protein G binding, or other capture molecule), by charge (e.g., ion-exchange chromatography), by size (e.g., size exclusion chromatograph), or otherwise. Purifying a population of immunoglobulins can comprise treating a composition comprising the population of immunoglobulins with one or more of acids, bases, salts, enzymes, heat, cold, coagulation factors, or other suitable agents. Purifying can further include adsorption of a composition comprising a target cell or molecule and an impurity onto non-target cells or molecules (e.g., red blood cells) to partially or completely remove the impurity. Purifying can further include pre-treatment of serum or plasma, e.g., caprylic acid fractionation.

The terms “treating”, and “treatment” refer to one or more of relieving, alleviating, delaying, reducing, reversing, improving, or managing at least one symptom of a condition in a subject. The term “treating” can also mean one or more of arresting, delaying the onset (i.e., the period prior to clinical manifestation of the condition) or reducing the risk of developing or worsening a condition.

The term “pharmaceutically acceptable” means biologically or pharmacologically compatible for in vivo use in animals or humans and can mean approved by a regulatory agency of the Federal or a state government or listed in the U.S. Pharmacopeia or other generally recognized pharmacopeia for use in animals, and more particularly in humans.

The term “hyperimmunized” refers to an immunization regimen that generates an immune response in the subject greater than required to produce a desired titer (e.g. a binding titer) after dilution of the immunoglobulin produced by the subject. For example, if a desired titer is 1:100, one can hyperimmunize an animal by a prime immunization followed by one, two, three or more boost immunizations to produce a 1:1,000 titer, or greater titer, in the subject, so that immunoglobulin produced by the subject can be diluted in the production of a biotherapeutic in order to give a desired titer in the biotherapeutic.

The term “affinity” refers to the strength of the interaction between an epitope and an antibody's antigen binding site. The affinity can be determined, for example, using the equation

K_{A}=[Ab−Ag]/[Ab][Ag]

Where K_{A}=affinity constant; [Ab]=molar concentration of unoccupied binding sites on the antibody; [Ag]=molar concentration of unoccupied binding sites on the antigen; and [Ab−Ag]=molar concentration of the antibody-antigen complex. The K_{A} describes how many antibody-antigen complexes exist at the point when equilibrium is reached. The time taken for this to occur depends on rate of diffusion and is similar for every antibody. However, high-affinity antibodies will bind a greater amount of antigen in a shorter period of time than low-affinity antibodies. The K_{A} of the antibodies produced can vary and range from between about 10⁵ mol⁻¹ to about 10¹² mol⁻¹ or more (e.g., a K_{A} can be about 10⁵ mol⁻¹, 10⁶ mol⁻¹, 10⁷ mol⁻¹, 10⁸ mol⁻¹, 10⁹ mol⁻¹, 10¹⁰ mol⁻¹, 10¹¹ mol⁻¹, or 10¹² mol⁻¹). The K_{A} can be influenced by factors including pH, temperature, and buffer composition.

Antibody affinity can be measured using any means commonly employed in the art, including but not limited to the use of biosensors, such as surface plasmon resonance (SPR). Resonance units are proportional to the degree of binding of soluble ligand to the immobilized receptor (or soluble antibody to immobilized antigen). Determining the amount of binding at equilibrium with different known concentrations of receptor (antibody) and ligand (protein antigen) allows the calculation of equilibrium constants (K_{A}, K_{D}), and the rates of dissociation and association (k_{off}, kₒₙ).

The term “avidity” refers to the accumulated strength of multiple affinities of individual non-covalent binding interactions, such as between an antibody and its antigen. Avidity is related to both the affinity of individual immunoglobulin molecules in the population with specific epitopes, and also the valences of the immunoglobulins and the antigen. For example, the interaction between a bivalent monoclonal antibody and an antigen with a highly repeating epitope structure, such as a polymer, would be one of high avidity. Avidity is measured by the off rate (k_{off}).

For example, K_{D} (the equilibrium dissociation constant) is a ratio of k_{off}/kₒₙ, between the antibody and its antigen. K_{D} and affinity are inversely related. The lower the K_{D} value (lower antibody concentration), the higher the affinity of the antibody. Most antibodies have K_{D} values in the low micromolar (10⁻⁶) to nanomolar (10⁻⁷ to 10⁻¹) range. High affinity antibodies are generally considered to be in the low nanomolar range (10⁻⁹) with very high affinity antibodies being in the picomolar (10⁻¹²) range or lower (e.g., 10⁻¹³ to 10⁻¹ range). In one embodiment, the antibodies produced by immunization with the HA-hFc antigen disclosed herein have a K_{D} ranging from about 10⁻⁶ to about 10⁻¹⁵, from about 10⁻⁷ to about 10⁻¹⁵, from about 10⁻⁸ to about 10⁻¹⁵, and from about 10⁻⁹ to about 10⁻¹⁵, from about 10⁻¹⁰ to about 10⁻¹⁵, about 10⁻¹¹ to about 10⁻¹⁵, about 10⁻¹² to about 10⁻¹⁵, about 10⁻¹³ to about 10⁻¹⁴, about 10⁻¹³ to about 10⁻¹⁵, and about 10⁻¹⁴ to about 10⁻¹⁵.

A population of human immunoglobulins produced by the methods disclosed herein, i.e., a population of ungulate-derived polyclonal human immunoglobulins, has high avidity, indicating the immunoglobulins bind tightly to the antigen. In one embodiment, the antibodies produced by immunization of an antigen containing an influenza HA protein tethered to an the Fc portion of a human immunoglobulin (HA-hFc) can have an avidity ranging from about 10⁻¹×1/sec to about 10⁻¹³×1/sec, from about 10⁻³×1/sec to about 10⁻¹³×1/sec, from about 10⁻⁵×1/sec to about 10⁻¹³×1/sec, from about 10⁻⁶×1/sec to about 10⁻¹³×1/sec, from about 10⁻⁷×1/sec to about 10⁻¹³×1/sec, from about 10⁻⁸×1/sec to about 10⁻¹³×1/sec, from about 10⁻⁹×1/sec to about 10⁻¹³×1/sec, from about 10⁻¹⁰×1/sec to about 10⁻¹³×1/sec, from about 10⁻¹¹×1/sec to about 10⁻¹×1/sec, or from about 10⁻¹²×1/sec to about 10⁻¹³×1/sec.

An ungulate-derived polyclonal human immunoglobulin is “specific to” or “specifically binds” (used interchangeably herein) to an influenza HA protein target. A molecule is said to exhibit “specific binding” if it reacts or associates more frequently, more rapidly, with greater duration and/or with greater affinity with a particular cell or substance than it does with alternative cells or substances. An ungulate-derived polyclonal human immunoglobulin compositions “specifically binds” to a particular protein or substance if it binds with greater affinity, avidity, more readily, and/or with greater duration than it binds to an alternative non-HA protein. For example, an immunoglobulin that specifically to influenza HA protein is an immunoglobulin that binds influenza HA protein with greater affinity, avidity, more readily, and/or with greater duration than it binds to other proteins.

The term “HAC vector” means a vector which comprises at least a human chromosome-derived centromere sequence, a telomere sequence, and a replication origin, and can contain any other sequences as desired for a given application. When present in a host cell, the HAC vector exists independently from a host cell chromosome in the nucleus. Any suitable method can be used to prepare HAC vectors and to insert nucleic acid molecules of interest into the HAC, including but not limited to those described in the examples that follow. An HAC vector can be a double stranded DNA vector.

Provided herein are methods of making ungulate-derived polyclonal human immunoglobulin compositions for treatment of influenza infection, comprising administering an antigen comprising a HA protein from influenza A and/or influenza B, or antigenic fragment thereof, or a polynucleotide encoding the antigen, to a transgenic ungulate, wherein the transgenic ungulate comprises a genome comprising a human immunoglobulin locus or an artificial chromosome comprising a human immunoglobulin locus, wherein the transgenic ungulate produces a population of polyclonal human immunoglobulins that specifically binds the HA.

In a variation, an HA protein from two or more strains of an influenza that can infect non-humans, or a polynucleotide encoding it, is used for immunization to produce ungulate-derived polyclonal non-human immunoglobulin compositions (e.g., a domesticated animal such as a dog, cat, sheep, etc.). The transgenic ungulate can in such cases comprise an artificial chromosome encoding an Ig locus of the non-human species such that antibodies of that species are generated.

### Preparation of Ungulate-Derived Polyclonal Human Immunoglobulin Compositions

In embodiments of the methods of the disclosure, the genome of the transgenic ungulate can comprise a human immunoglobulin locus. Illustrative methods are provided in U.S. Pat. Nos. 9,902,970; 9,315,824; 7,652,192; and 7,429,690; and 7,253,334, the disclosure of which are incorporated by reference herein for all purposes. Further illustrative methods are provided by Kuroiwa, Y., et al. (2009) Nat Biotechnol. 27(2):173-81, and Matsushita et al. (2015) PLoS ONE 10(6):e0130699, which are incorporated herein by reference.

A human artificial chromosome (HAC) vector can comprise genes encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;

wherein at least one class switch regulatory element of the genes encoding the one or more human antibody heavy chains is replaced with an ungulate-derived class switch regulatory element. HAC vectors can be used, for example, for large-scale production of fully human antibodies by transgenic animals. A HAC vector can comprise one or more genes encoding a human antibody heavy chain. Any human antibody heavy chain or combinations of human antibody heavy chains in combination can be encoded by one or more nucleic acid molecules on the HAC. In various embodiments, 1, 2, 3, 4, 5, 6, 7, 8, or all 9 of human antibody heavy chains IgM, IgG1, IgG2, IgG3, IgG4, IgA1, IgA2, IgE and IgD can be encoded on the HAC vector in one or more copies. In one embodiment, a HAC vector comprises a human IgM antibody heavy chain encoding gene, alone or in combination with 1, 2, 3, 4, 5, 6, 7, or the other 8 human antibody chain encoding genes. In one aspect, a HAC vector comprises a gene encoding at least a human IgG1 antibody heavy chain; in this embodiment, a HAC vector can comprise a gene encoding a human IgM antibody heavy chain or a gene encoding a human IgM antibody heavy chain that has been chimerized to encode an ungulate-derived IgM heavy chain constant region (such as a bovine heavy chain constant region). In another embodiment, a HAC vector comprises a gene encoding at least a human IgA antibody heavy chain; in this embodiment, a HAC vector can comprise a gene encoding a human IgM antibody heavy chain or a gene encoding a human IgM antibody heavy chain that has been chimerized to encode an ungulate-derived IgM heavy chain constant region (such as a bovine heavy chain constant region). In another aspect, a HAC vector comprises genes encoding all 9 antibody heavy chains where the gene encoding a human IgM antibody heavy chain has been chimerized to encode an ungulate-derived IgM heavy chain constant region. In another embodiment, an HAC vector can comprise a portion of human chromosome 14 that encodes the human antibody heavy chains. The variable region genes and the constant region genes of the human antibody heavy chain form a cluster and the human heavy chain locus is positioned at 14q32 on human chromosome 14. In one embodiment, the region of human chromosome 14 inserted into a HAC vector comprises the variable region and the constant region of the human antibody heavy chains from the 14q32 region of human chromosome 14.

In some embodiments, at least one class switch regulatory element of the human antibody heavy chain encoding nucleic acid molecule is replaced in a HAC vector with an ungulate-derived class switch regulatory element. A class switch regulatory element refers to a nucleic acid molecule that is 5′ to an antibody heavy chain constant region. Each heavy chain constant region gene is operatively linked with (i.e., under control of) its own switch region, which is also associated with its own I-exons. Class switch regulatory elements regulate class switch recombination and determine Ig heavy chain isotype. Germline transcription of each heavy chain isotype is driven by the promoter/enhancer elements located just 5′ of the I-exons and those elements are cytokine- or other activator-responsive. In a simple model of class switch, the specific activators and/or cytokines induce each heavy chain isotype germline transcription from its class switch regulatory element (i.e., activator/cytokine-responsive promoter and/or enhancer). Class switch is preceded by transcription of I-exons from each Ig heavy (IGH) locus-associated switch region. Each heavy chain constant region gene is linked with its own switch region.

Any suitable ungulate-derived class switch regulatory element can be used. For example, the human heavy chain gene isotypes listed below have the following class switch regulatory elements:
IgM: Iμ-Sμ,
IgG1: Iγ1-Sγ1,
IgG2: Iγ2-Sγ2,
IgG3: Iγ3-Sγ3,
IgG4: Iγ4-Sγ4,
IgA1: Iα1-Sα1,
IgA2: Iα2-Sα2, and
IgE: Iε-Sε.

In various embodiments, 1, more than 1, or all of the human antibody heavy chain genes in an HAC vector have their class switch regulatory element replaced with an ungulate-derived class switch regulatory element, including but not limited to ungulate Iμ-Sμ, Iγ-Sγ, Iα-Sα, or Iε-Sε, class switch regulatory elements. In one embodiment, an Iγ1-Sγ1 human class switch regulatory element for human IgG1 heavy chain encoding nucleic acid molecule on the HAC vector (e.g., such as that in SEQ ID NO: 183 of U.S. Pat. No. 9,902,970) is replaced with an ungulate Iγ1-Sγ1 class switch regulatory element. Exemplary ungulate Iγ1-Sγ1 class regulatory switch elements include a bovine IgG1 Iγ1-Sγ1 class switch regulatory element (e.g., see SEQ ID NO: 182 of U.S. Pat. No. 9,902,970), a horse Iγ1-Sγ1 class switch regulatory element (e.g., see SEQ ID NO: 185 of U.S. Pat. No. 9,902,970), and a pig Iγ1-Sγ1 class switch regulatory element (e.g., see SEQ ID: 186 of U.S. Pat. No. 9,902,970). However, it is not necessary to replace the human class switch regulatory element with an ungulate class switch regulatory element from the corresponding heavy chain isotype. Thus, for example, an Iγ3-Sγ3 human class switch regulatory element for a human IgG3 heavy chain encoding nucleic acid molecule on the HAC vector can be replaced with an ungulate Iγ1-Sγ1 class switch regulatory element. Any such combination can be used in the HAC vectors described herein.

In another embodiment, a HAC vector comprises at least one ungulate enhancer element to replace an enhancer element associated with one or more human antibody heavy chain constant regions encoding nucleic acid molecules on the HAC. There are two 3′ enhancer regions (Alpha 1 and Alpha 2) associated with human antibody heavy chain genes. Enhancer elements are 3′ to the heavy chain constant region and also help regulate class switch. Any suitable ungulate enhancer can be used, including but not limited to 3′Eα enhancers. Non-limiting examples of 3′Eα enhancers that can be used include 3′Eα, 3′Eα1, and 3′Eα2. Exemplary 3′Eα enhancer elements from bovine that can be used in the HACs and replace the human enhancer include but are not limited to bovine HS3 enhancer (e.g., see SEQ ID NO: 190 of U.S. Pat. No. 9,902,970), bovine HS12 enhancer (e.g., see SEQ ID NO: 191 of U.S. Pat. No. 9,902,970), and bovine enhancer HS4. The enhancers can be used, for example, wherein a HAC vector comprises the variable region and the constant region of the human antibody heavy chains from the 14q32 region of human chromosome 14.

HAC vectors can comprise one or more genes encoding a human antibody light chain. Any suitable human antibody light chain-encoding genes can be used in the HAC vectors. The human antibody light chain includes two types of genes, i.e., the kappa/K chain gene and the lambda/L chain gene. In one embodiment, a HAC vector comprises genes encoding both kappa and lambda, in one or more copies. The variable region and constant region of the kappa chain are positioned at 2p11.2-2p12 of the human chromosome 2, and the lambda chain forms a cluster positioned at 22q11.2 of the human chromosome 22. Therefore, in one embodiment, a HAC vector comprises a human chromosome 2 fragment containing the kappa chain gene cluster of the 2p11.2-2p12 region. In another embodiment, the HAC vectors of the present invention comprise a human chromosome 22 fragment containing the lambda chain gene cluster of the 22q11.2 region.

In another embodiment, an HAC vector comprises at least one gene encoding a human antibody surrogate light chain. The gene encoding a human antibody surrogate light chain refers to a gene encoding a transient antibody light chain which is associated with an antibody heavy chain produced by a gene reconstitution in the human pro-B cell to constitute the pre-B cell receptor (preBCR). Any suitable human antibody surrogate light chain encoding gene can be used, including but not limited to the VpreB1 (e.g., see SEQ ID NO: 154 of U.S. Pat. No. 9,902,970), VpreB3 (e.g., see SEQ ID NO: 178 of U.S. Pat. No. 9,902,970) and λ5 (also known as IgLL1, e.g., see SEQ ID NO: 157 of U.S. Pat. No. 9,902,970) human antibody surrogate light chains, and combinations thereof. The VpreB gene and the λ5 gene are positioned within the human antibody lambda chain gene locus at 22q11.2 of the human chromosome 22. Therefore, a HAC vector can comprise the 2211.2 region of human chromosome 22 containing the VpreB gene and the λ5 gene. The human VpreB gene provides either or both of the VpreB1 gene (e.g., see SEQ ID NO: 154 of U.S. Pat. No. 9,902,970) and the VpreB3 (e.g., see SEQ ID NO: 178 of U.S. Pat. No. 9,902,970) gene and in one embodiment provides both of the VpreB1 gene and the VpreB3 gene.

In yet another embodiment, the HAC vector comprises a gene encoding an ungulate-derived IgM heavy chain constant region. In this embodiment, the IgM heavy chain constant region is expressed as a chimera with the human IgM antibody heavy chain variable region. Any suitable ungulate IgM heavy chain antibody constant region encoding nucleic acid molecule can be used, including but not limited to bovine IgM, (e.g., see SEQ ID NO: 10), horse IgM, (e.g., see SEQ ID NO: 176 of U.S. Pat. No. 9,902,970), sheep IgM, (e.g., see SEQ ID NO: 174 of U.S. Pat. No. 9,902,970), and pig IgM, (e.g., see SEQ ID NO: 175 of U.S. Pat. No. 9,902,970). In one embodiment, the chimeric IgM comprises the sequence in for e.g., SEQ ID NO: 200 of U.S. Pat. No. 9,902,970. Pre-BCR/BCR signaling through the IgM heavy chain molecule promotes proliferation and development of the B cell by interacting with the B cell membrane molecule Ig-alpha/Ig-beta to cause a signal transduction in cells. The transmembrane region and/or other the constant region of IgM are considered to have important roles in the interaction with Ig-alpha/Ig-beta for signal transduction. Examples of the IgM heavy chain constant regions include nucleic acid molecules encoding constant region domains such as CH1, CH2, CH3, and CH4, and the B-cell transmembrane and cytoplasmic domains such as TM1 and TM2. The nucleic acid molecule encoding an ungulate-derived IgM heavy chain constant region which is comprised in the human artificial chromosome vector of the invention is not particularly limited so long as the region is in a range which can sufficiently induce the signal of the B-cell receptor or B-cell proliferation/development in the above-described IgM heavy chain constant region. In one embodiment, a nucleic acid molecule encoding an ungulate-derived IgM heavy chain constant region provides a transmembrane and cytoplasmic TM1 domain and TM2 domain derived from an ungulate, and in other embodiments nucleic acid molecules encode the ungulate-derived CH2 domain, CH3 domain, CH4 domain, TM1 domain, and TM2 domain or the ungulate-derived CH1 domain, CH2 domain, CH3 domain, CH4 domain, TM1 domain, and TM2 domain.

In one embodiment, a gene encoding the IgM heavy chain constant region of the bovine is a gene encoding a bovine IgM heavy chain constant region which is included in an IGHM region at which a bovine endogenous IgM heavy chain gene is positioned (derived from IGHM) or a gene encoding a bovine IgM heavy chain constant region in an IGHML1 region (derived from IGHML1). In another embodiment, a gene encoding a bovine IgM heavy chain constant region is included in the IGHM region.

In a further embodiment, a HAC vector comprises a gene encoding a human antibody heavy chain comprises a gene encoding a human heavy chain (for example, a human IgG heavy chain, such as an IgG1 heavy chain), and wherein a transmembrane domain and an intracellular domain of a constant region of the human heavy chain gene are replaced with a transmembrane domain and an intracellular domain of an ungulate-derived heavy chain (for example, an ungulate IgG heavy chain, such as an IgG1 heavy chain), constant region gene. In one embodiment, a gene encoding the transmembrane domain and the intracellular domain of an ungulate-derived (such as bovine) IgG (such as IgG1) heavy chain constant region are used to replace the corresponding regions of the human IgG heavy chain gene. In another embodiment, a gene encoding the TM1 and TM2 domains of an ungulate-derived (such as bovine) IgG (such as IgG1) heavy chain constant region are used to replace the corresponding regions of a human IgG heavy chain gene. In another embodiment, the gene encoding the one or more of the CH1-CH4 domains and/or the TM1 and TM2 domains of an ungulate-derived (such as bovine) IgG (such as IgG1) heavy chain constant region are used to replace the corresponding regions of the human IgG heavy chain gene.

Also provided are transgenic ungulates comprising a HAC vector according to any embodiment or combination of embodiments of the disclosure. A transgenic ungulate comprising a HAC vector refers to an animal into which a human artificial chromosome vector as described herein is introduced. A transgenic ungulate having a HAC vector is not particularly limited and can be, for example, cows, horses, goats, sheep, and pigs. In one aspect, a transgenic ungulate is a bovine. In some embodiments, ungulates bearing the HAC vectors containing human immunoglobulin locus are referred to as transchromosomic (Tc) ungulates. In some embodiments, the ungulates can be cows. In some embodiments, cows bearing the HAC vectors containing human immunoglobulin locus are referred to as transchromosomic (Tc) bovines. A transgenic ungulate having a HAC vector of as described herein can be constructed, for example, by introducing a HAC vector into an oocyte of a host animal using any suitable technique, such as those described herein. The HAC vector of the present invention can, for example, be introduced into a somatic cell derived from a host ungulate by a microcell fusion method. Thereafter, an animal having an HAC vector can be constructed by transplanting a nucleus or chromatin agglomerate of the cell into an oocyte and transplanting the oocyte or an embryo to be formed from the oocyte into the uterus of a host animal to give birth. It can be confirmed by a method of Kuroiwa et al. (Kuroiwa et al., Nature Biotechnology, 18, 1086-1090, 2000 and Kuroiwa et al., Nature Biotechnology, 20, 889-894) whether an animal constructed by the above method has the human artificial chromosome vector.

The disclosure further provides transgenic ungulates comprising genes integrated into their genome encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;

wherein at least one class switch regulatory element of the genes encoding the one or more human antibody heavy chains is replaced with an ungulate-derived class switch regulatory element.

In such embodiments, a transgenic ungulate can include any of the nucleic acid molecules as described herein for the HAC vector, but rather than being present in a HAC vector, the nucleic acid molecules are integrated into a chromosome of the ungulate.

The disclosure further provides methods of producing an ungulate-derived polyclonal human immunoglobulin composition. A method can comprise: (a) administering influenza HA protein from two or more influenza strains, to groups of transgenic ungulates to produce and accumulate a population of human immunoglobulins specific to influenza HA protein (or T cells, B cells, and/or monocytes) in the serum or plasma of the ungulates; and (b) isolating, recovering, and/or purifying the population of human immunoglobulins specific to the influenza HA protein (or T cells, B cells, and/or monocytes) from the serum or plasma of the different groups of the transgenic ungulate and pooling the population of human immunoglobulins to prepare the ungulate-derived polyclonal immunoglobulin compositions.

In some embodiments, the methods of producing an ungulate-derived polyclonal human immunoglobulin composition can involve administering (also herein immunizing) an ungulate with an influenza HA protein from two or more strains. The influenza HA protein can be a quadrivalent seasonal influenza HAG protein vaccine that is prepared using recombinant DNA technology. In some embodiments, influenza HA proteins from one, two, three, four, five, six or more strains can be administered to an ungulate to generate compositions described herein. In some embodiments, the two or more strains administered to an ungulate may be seasonal variants from the same year or different years. The influenza HA protein from two or more strains of influenza can be administered as a single dose form or influenza protein from each strain can be administered separately. In some embodiments, each of the influenza HA proteins is expressed an insect cell line using a baculovirus vector or in eggs or egg cells, extracted from the eggs or cells with, e.g., Triton X-100 and further purified by column chromatography. The purified influenza HA proteins can then be blended and filled into single-dose syringes. The amount of each influenza HA protein in a single dose syringe can be about 0.1 to 10 mg (e.g., about 0.1, 1, 2, 3, 4, 5, 6, 7, 8, 9, 10 mg or more), for example about 0.45 to 0.9 milligrams. In some embodiments, the influenza HA proteins are administered to the ungulates as a solution containing sodium chloride (e.g., about 1, 2, 3, 4, 4.4, 5, 6, 7 mg or more), monobasic sodium phosphate (e.g., about 0.01, 0.1, 0.2, 0.3, 0.4, 0.5, 0.6, 0.7 mg or more), dibasic sodium phosphate (e.g., about 0.1, 0.2, 0.3, 0.4, 0.5, 0.6, 0.7, 0.8, 0.9 mg or more), and polysorbate (e.g., TWEEN® 20) (e.g., about 15, 20, 25, 27.5, 30, 35 mcg or more). In some embodiments, the influenza HA protein can be a quadrivalent seasonal influenza HAG protein vaccine. In some embodiments, the HAG protein vaccine can be obtained from more than one strain of influenza. In some embodiments, the same transgenic ungulates can be repeatedly immunized with a multiple yearly seasonal influenza HAG protein vaccines recommended by the World Health Organization. In some embodiments, the influenza HAG protein vaccine can be prepared from influenza strains in Table 1.

TABLE 1
Seasonal Influenza Vaccine Strains

| | H1N1 | H3N2 | B-Vic | B-Yam |
|---|---|---|---|---|
| 2018- | A/Michigan/45/2015 | A/Singapore/INIFMH-16- | B/Maryland/15/2016 | B/Phuket/3073/2013 |
| 19 | | 0019/2016 | | |
| 2019- | A/Brisbane/02/2018 | A/Kansas/14/2017 | B/Colorado/06/2017 | B/Phuket/3073/2013 |
| 20 | | | | |
| 2020- | A/Guangdong- | A/Hong Kong/45/2019 | B/Washington/02/2019 | B/Phuket/3073/2013 |
| 21 | maonan/2019 | | | |
| 2021- | A/Victoria/2570/19 | A/Cambodia/e0826360 | B/Washington/02/2019 | B/Phuket/3073/2013 |
| 22 | | | | |

Methods of making ungulate-derived human polyclonal immunoglobulin for treatment of a particular indication can be optimized to yield immunoglobulins that are immunogenic, effective, and safe to administer to subject. The inventors have assessed antigens that are active and/or inactivated whole cells (bacteria, viruses and human cells), split virion antigens, partial and/or full-length recombinant viral glycoproteins, and partial and/or full-length viral glycoprotein nucleic acid molecules for influenza. Not all antigens tested for a particular indication yield polyclonal immunoglobulins with desired properties. For influenza, the inventors utilized two distinct antigen preparations to immunize ungulates and found that immunization of ungulates with quadrivalent rHA0 proteins resulted in human polyclonal immunoglobulins with desired safety and immunogenicity profiles suitable for further testing in clinical contexts.

In some embodiments, a group of transgenic ungulates can be divided into several sub-groups with each sub-group being immunized with two or more, up to 4, up to 5, up to 6, up to 7 or more seasonal variants or sets of seasonal variants.

In some embodiments, HA protein(s) from two or more strains of influenza, or a polynucleotide encoding the HA protein from two or more strains of influenza, are administered before, during, or after administration of one or more adjuvants. In some embodiments, the antigen and one or more adjuvants are administered together in a single composition, comprising optionally one or more pharmaceutically acceptable excipients.

Illustrative adjuvants include an aluminum salt adjuvant, an oil in water emulsion (e.g., an oil-in-water emulsion comprising squalene, such as MF59 or AS03), a TLR7 agonist (such as imidazoquinoline or imiquimod), or a combination thereof. Suitable aluminum salts include hydroxides (e.g., oxyhydroxides), phosphates (e.g., hydroxyphosphates, orthophosphates), (e.g., see chapters 8 & 9 of Vaccine Design. (1995) eds. Powell & Newman. ISBN: 030644867X. Plenum), or mixtures thereof. Further illustrative adjuvants include, but are not limited to, Adju-Phos, Adjumerlm, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, MONTANIDE™ ISA-25, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL.TM., MPL-SE, MTP-PE, MTP-PE Liposomes, Murametide, Murapalmitine, NAGO, nCT native Cholera Toxin, Non-Ionic Surfactant Vesicles, non-toxic mutant E1 12K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, ISA-25/Quil-A, Rehydragel HPA, Rehydragel LV, RIBI, Ribilike adjuvant system (MPL, TMD, CWS), S-28463, SAB-adj-1, SAB-adj-2, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes.

Immunization can be carried out by administering the antigen with, for example, a complete Freund's adjuvant or an appropriate adjuvant such as an aluminum hydroxide gel, and pertussis bacteria vaccine, intramuscularly, intranasally, orally, subcutaneously, intravenously, or intraperitoneally into a transgenic ungulate. In one embodiment, immunization comprises hyperimmunization.

In various embodiments, influenza HA protein from two or more strains of influenza (with or without adjuvant) is administered once to 10 times (e.g., 1, 2, 3, 4, 5, 6, 7, 8, 9, 10, or more times) every 1 to 4 weeks (e.g., 1, 2, 3, 4, or more weeks) after the first administration. After 1 to 14 days (e.g., 1, 2, 3, 4, 5, 6, 7, 8, 9, 10, 11, 12, 13, 14, or more) from each administration, blood is collected from the animal to measure the antibody value of the serum. As a non-limiting example, the blood can be collected from the animal after 5 immunizations to measure the antibody value of the serum.

In some embodiments, the influenza HA protein from two or more strains is administered 3, 4, 5, 6 or more times. Administration of the influenza HA protein from two or more strains can be performed, e.g., every 1-2 weeks, 2-3 weeks, 3-4 weeks, 4-5 weeks, 5-6 weeks, or 6-7 weeks, or longer intervals, e.g., every 1 week, 2 weeks, 3 weeks, 4 weeks, 5 weeks or 6 weeks. After each immunization, serum and/or plasma can be harvested from the transgenic ungulate one or more times. For example, methods can include performing control bleeds two or three times at intervals about 7-14 days. In some embodiments, the influenza HA protein can be administered every month, every 2 months, every 3 months, every 4 months, every 5 months, every 6 months, every 7 months, every 8 months, every 9 months, every 10 months, every 11 months, every 12 months or more.

HA-specific human immunoglobulin compositions (such as HA-specific ungulate-derived polyclonal human immunoglobulin compositions) can be produced by immunizing the transgenic ungulate having the HAC vector with influenza HA, or another antigen of the disclosure, to produce the HA-specific polyclonal human immunoglobulin in the serum or plasma of the transgenic ungulate and recovering the HA-specific polyclonal human immunoglobulin from the serum or plasma of the transgenic ungulate.

In a variation, the methods of the disclosure are used to generate a monoclonal antibody. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Kohler and Milstein, Nature 256:495 (1975)). An example of a preparation method for hybridomas comprises the following steps of: (1) immunizing a transgenic ungulate with a recombinant HA comprising two or more strains of influenza; (2) collecting antibody-producing cells from the transgenic ungulate (i.e. from lymph nodes); (3) fusing the antibody-producing cells with myeloma cells; (4) selecting hybridomas that produce a monoclonal antibody specific to influenza HA protein from the fused cells obtained in the above step; and optionally (5) selecting a hybridoma that produces monoclonal antibodies specific to influenza HA protein from two or more strains of influenza from the selected hybridomas.

Methods for detecting and measuring the HA-specific ungulate-derived polyclonal human immunoglobulins in a composition can include a binding assay by an enzyme-linked immunosorbent assay, and the like. The binding amount of a human immunoglobulin can be measured by incubating the composition comprising the human immunoglobulin with cells (e.g., T cells, B cells and/or monocytes, or recombinant protein antigen(s)), and then using an antibody specifically recognizing human immunoglobulin. In some embodiments, the method includes collecting the polyclonal serum and/or polyclonal plasma from the transgenic ungulate and pooling polyclonal serum/plasma from different transgenic ungulates that have been immunized with influenza HA0 protein from two or more influenza strains. In some embodiments, the ungulate is a bovine or a goat. In some embodiments, the ungulate-derived polyclonal human immunoglobulin compositions comprise a population of fully human immunoglobulins. In some embodiments, the ungulate-derived polyclonal human immunoglobulin compositions comprise a population of fully human immunoglobulins, which is substantially human immunoglobulins.

In some embodiments, the population of fully human or substantially human immunoglobulins are purified from the serum of the transgenic ungulate after immunization. The term “purification” or “purify” as used herein, can refer to separating ungulate-derived polyclonal immunoglobulins from other substances present in the plasma or serum.

Plasma can be collected using, for example, an automated plasmapheresis system. After collection of sufficient volume, plasma can be frozen and stored. Frozen plasma can be thawed, pooled, fractionated by caprylic acid and clarified by depth filtration in the presence of filter aid. The clarified sample containing human immunoglobulin G (hIgG) can be further purified by affinity chromatography, first using an anti-human IgG kappa affinity column to capture hIgG pAbs and to remove residual non-hIgG and bovine plasma proteins. The sample can be subsequently passed through an anti-bovine IgG heavy chain specific affinity column to further remove residual IgG molecules that contain a bovine heavy chain. The hIgG fraction can then be subjected to a Q Sepharose chromatography polishing step to further reduce impurities, nanofiltration, final buffer exchange, concentration, and sterile filtration. The ungulate-derived human polyclonal immunoglobulin composition can be filtered and filled into vials.

Immunoglobulins can also be purified by fractionation of plasma, by affinity (e.g., protein A or protein G binding, or other capture molecule), by charge (e.g., ion-exchange chromatography), by size (e.g. size exclusion chromatograph), or otherwise. Purifying can comprise treating plasma or serum with one or more of acids, bases, salts, enzymes, heat, cold, coagulation factors, or other suitable agents. Ungulate-derived human polyclonal immunoglobulins can be fractionated by caprylic acid (CA) and clarified by depth filtration. Clarified material containing ungulate-derived human immunoglobulin G (IgG) can be purified by affinity chromatography, first using an anti-human IgG affinity column to bind ungulate-derived human IgG (hlgG) and remove bovine plasma proteins (BPP) followed by a low pH treatment for viral inactivation, and then, by passing through an anti-bovine IgG (blgG) heavy chain (HC) specific affinity column to further remove residual IgG molecules that contain a bovine HC or Fc of bovine HC.

The polyclonal serum or plasma, or human immunoglobulin purified from the polyclonal serum or plasma, can be pooled and used as an ungulate-derived polyclonal human immunoglobulin compositions for treatment of viral infection, for example influenza infection.

The methods have the surprising advantage that the HA-specific immunoglobulins (such as HA-specific ungulate-derived polyclonal human immunoglobulin compositions) are produced in high yield, in high purity, and/or as a high percentage of total immunoglobulin present in the serum or plasma of the transgenic ungulate. Additionally, by pooling immunoglobulins obtained from transgenic ungulates immunized with different seasonal strains, the compositions of the disclosure can specifically target influenza over time despite the evolution of the virus.

Furthermore, some embodiments produce HA-specific ungulate-derived polyclonal human immunoglobulin compositions having glycans that comprise at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95% or higher percentage of fucosylated glycans.

In a variation, a method is provided of recovering a human antibody comprising: (i) isolating lymphocytes from the transgenic ungulate; (ii) generating a human monoclonal antibody producing hybridoma from the lymphocytes; and (iii) recovering human monoclonal antibody specific to the antigen from the hybridoma. In another embodiment, the lymphocytes from the transgenic ungulate are isolated from lymph nodes of the transgenic ungulate. In a further embodiment the transgenic ungulate is hyperimmunized with the target antigen.

### Ungulate-Derived Polyclonal Human Immunoglobulin Compositions

In some embodiments, the ungulate-derived polyclonal human immunoglobulin compositions can be prepared by combining one or more lots of human immunoglobulins obtained from immunizing transgenic ungulates with multiple yearly seasonal influenza HAG protein vaccines. Each lot may be prepared by immunizing a group of transgenic ungulates with two or more seasonal influenza variants. The same group of transgenic ungulates may be immunized in subsequent years with different seasonal influenza variants to prepare a second lot. In some embodiments, the ungulate-derived polyclonal human immunoglobulin compositions can include one, two, three, four, five or more lots with each lot comprising about 5%, 10%, 15%, 20%, 25%, 30%, 35%, 40%, 45%, 50%, 55%, 60%, 65%, 70%, 75%, 80%, 85%, 90% or more of the ungulate-derived polyclonal human immunoglobulin compositions. In some embodiments, a population of ungulate-derived polyclonal human immunoglobulin compositions comprise glycans covalently linked to the human immunoglobulins. In some embodiments, the glycans can be N-Glycolylneuraminic acid (NGNA) and/or N-Acetylneuraminic acid (NANA) moieties. Naturally occurring human immunoglobulin G, isolated from humans comprise N-Acetylneuraminic acid (NANA) moieties only. Ungulate-derived polyclonal human immunoglobulin compositions, in contrast, can comprise both NANA-bearing glycan moieties and N-Glycolylneuraminic acid (NGNA)-bearing glycan moieties. In some embodiments, the percentage of glycans that are N-Acetylneuraminic acid (NANA) moieties in ungulate-derived polyclonal human immunoglobulin compositions is about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more. In some embodiments, the percentage of N-Glycolylneuraminic acid (NGNA)-bearing glycans is about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more.

In some embodiments, an ungulate-derived polyclonal human immunoglobulin composition comprises at least about 10%, 20%, 30%, 40%, 50%, 60%, 70%, at least about 80%, or at least about 90% N-Glycolylneuraminic acid (NGNA)-bearing glycans. In some embodiments, an ungulate-derived polyclonal human immunoglobulin composition comprises at least about 10%, 20%, 30%, 40%, 50%, 60%, 70%, at least about 80%, or at least about 90% NANA-bearing glycans. In some embodiments, an ungulate-derived polyclonal human immunoglobulin composition comprises less than 100%, less than 90%, less than 80%, less than 70%, less than 60%, less than 50%, less than 40%, less than 30%, less than 20%, less than 10%, less than 5%, or less than 1%.

In some embodiments, an ungulate-derived polyclonal human immunoglobulin composition comprises about 90% NGNA and about 10% NANA.

Furthermore, some embodiments produce HA-specific ungulate-derived polyclonal human immunoglobulin compositions having at most about the same ADCC or CDC activity as a reference immunoglobulin preparation, e.g., human-derived immunoglobulin.

In some embodiments, the population of ungulate-derived polyclonal human immunoglobulin compositions binds FcγRI with a K_{D} of 15 nM or greater. In some embodiments, the population of human immunoglobulins binds FcγRIIa with a K_{D} of 500 nM or greater. In some embodiments, the population of human immunoglobulins binds FcγRIIb/c with a K_{D} of 1 μM or greater. In some embodiments, the population of human immunoglobulins binds FcγRIIIa with a K_{D} of 1 μM or greater. In some embodiments, the population of human immunoglobulins binds FcγRIIIa with a K_{D} of 1 nM or greater.

In some embodiments a polyclonal serum or polyclonal plasma comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal serum or polyclonal plasma.

In some embodiments of the methods and compositions of the disclosure, a polyclonal serum or polyclonal plasma comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal serum or polyclonal plasma.

In some embodiments of the methods and compositions of the disclosure, a polyclonal serum or polyclonal plasma comprises 0-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal serum or polyclonal plasma.

In some embodiments of the methods and compositions of the disclosure, a polyclonal serum or polyclonal plasma comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal serum or polyclonal plasma.

In some embodiments of the methods and compositions of the disclosure, a polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, a polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, a polyclonal serum or polyclonal plasma comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal serum or polyclonal plasma.

In some embodiments of the methods and compositions of the disclosure, a polyclonal serum or polyclonal plasma comprises 1-4%, 2-5%, 3-6%, 4-7%, 5-8%, 6-9%, or 7-10% fully human (or substantially human) immunoglobulin by mass of total immunoglobulin in the polyclonal serum or polyclonal plasma.

In some embodiments of the methods and compositions of the disclosure, a polyclonal immunoglobulin comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal immunoglobulin.

In some embodiments of the methods and compositions of the disclosure, a polyclonal immunoglobulin comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal immunoglobulin.

In some embodiments of the methods and compositions of the disclosure, a polyclonal immunoglobulin comprises 0-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal immunoglobulin.

In some embodiments of the methods and compositions of the disclosure, a polyclonal immunoglobulin comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal immunoglobulin.

In some embodiments of the methods and compositions of the disclosure, a polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, a polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, a polyclonal immunoglobulin comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal immunoglobulin.

In some embodiments of the methods and compositions of the disclosure, a polyclonal immunoglobulin comprises 1-4%, 2-5%, 3-6%, 4-7%, 5-8%, 6-9%, or 7-10% fully human (or substantially human) immunoglobulin by mass of total immunoglobulin in the polyclonal immunoglobulin.

In some embodiments of the methods and compositions of the disclosure, a polyclonal immunoglobulin comprises at least 5% fully human immunoglobulin by mass of total immunoglobulin in a polyclonal immunoglobulin.

In some embodiments of the methods and compositions of the disclosure, a polyclonal immunoglobulin comprises 2% to 5% fully human immunoglobulin by mass of total immunoglobulin in the polyclonal immunoglobulin.

In some embodiments, ungulate-derived polyclonal human immunoglobulin compositions comprise “chimeric” human immunoglobulin having a human heavy chain and an ungulate kappa light chain. In one embodiment, the chimeric human immunoglobulin can be a chimeric immunoglobulin G (chimeric IgG, also herein termed “cIgG”). In some embodiments, a polyclonal immunoglobulin comprises less than about 0.5%, less than about 0.75%, less than about 1.0%, less than about 1.25%, less than about 1.5%, less than about 1.75%, less than about 2.0%, less than about 2.25%, less than about 2.5%, less than about 2.75%, less than about 3.0%, less than about 3.25%, less than about 3.5%, less than about 3.75%, or less than about 4.0% cIgG as a percent of total protein concentration. In some embodiments, the polyclonal immunoglobulin comprises about 0.5% to about 1.0%, about 1.0% to about 1.5%, about 1.5% to about 2.0%, about 1.5% to about 2.0%, about 2.0% to about 2.5%, or about 2.5% to about 3.0% cIgG as a percent of total protein concentration. In some embodiments, the polyclonal immunoglobulin comprises about 0.5% to about 1.0%, about 1.0% to about 2.0%, or about 1.0 to about 3.0% cIgG as a percent of total protein concentration.

In some embodiments, a chimeric human immunoglobulin can be chimeric immunoglobulin M (herein termed “cIgM”). In some embodiments, polyclonal immunoglobulin comprises less than about 0.5%, less than about 0.75%, less than about 1.0%, less than about 1.25%, less than about 1.5%, less than about 1.75%, less than about 2.0%, less than about 2.25%, less than about 2.5%, less than about 2.75%, less than about 3.0%, less than about 3.25%, less than about 3.5%, less than about 3.75%, or less than about 4.0% cIgM as a percent of total protein concentration. In some embodiments, the polyclonal immunoglobulin comprises about 0.5% to about 1.0%, about 1.0% to about 1.5%, about 1.5% to about 2.0%, about 1.5% to about 2.0%, about 2.0% to about 2.5%, or about 2.5% to about 3.0% cIgM as a percent of total protein concentration. In some embodiments, the polyclonal immunoglobulin comprises about 0.5% to about 1.0%, about 1.0% to about 2.0%, or about 1.0 to about 3.0% cIgM as a percent of total protein concentration. In one aspect, the cIgM can be removed from the polyclonal immunoglobulin compositions by a purification process.

In some embodiments, a polyclonal immunoglobulin comprises more than about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1% cIgG or cIgM, but less than about 1.5%, 2.0%, 2.5% 3%, 3.5%, 4.0%, 4.5%, or 5% cIgG or cIgM.

In some embodiments, ungulate-derived polyclonal human immunoglobulin compositions comprise at least about 70% IgG1. In some embodiments, human polyclonal immunoglobulins comprise less than about 30% IgG2. In some embodiments, human polyclonal immunoglobulins comprise less than about 4% IgG3 and/or IgG4.

In some embodiments, ungulate-derived polyclonal human immunoglobulin compositions comprise about 90% IgG1, about 10% IgG2 and less than 10% (e.g., less than 9, 8, 7, 6, 5, 4, 3, 2 1) of IgG3 and/or IgG4.

In some embodiments, polyclonal immunoglobulins of the disclosure can have a HAI titer of at least 64, 128, 256, 512, 1024, 2048, 4096, 8192, 16384 or more. As used herein, the HAI titer can be defined as the reciprocal of the highest dilution of antibody test article that inhibited RBC hemagglutination by the selected influenza virus.

In some embodiments, ungulate-derived polyclonal human immunoglobulin compositions of the disclosure can block influenza HA protein from binding to sialic acid. In other aspects, ungulate-derived polyclonal human immunoglobulin compositions can reduce binding of influenza HA protein to sialic acid by about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or 90-100%. In some embodiments, the ungulate-derived polyclonal human immunoglobulin compositions can reduce binding of influenza HA protein to sialic acid by about 10%, 20%, 30%, 40%, 50%, 60%, 70%, at least about 80%, or at least about 90%.

In some embodiments, ungulate-derived polyclonal human immunoglobulin compositions of the disclosure can block, Influenza A and/or Influenza B from infecting a mammalian cell, either partially or completely. In other aspects, ungulate-derived polyclonal human immunoglobulin compositions can block Influenza A and/or Influenza B from infecting a mammalian cell by about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or 90-100%. In some embodiments, the ungulate-derived polyclonal human immunoglobulin compositions can block Influenza A and/or Influenza B from infecting a mammalian cell by about 10%, 20%, 30%, 40%, 50%, 60%, 70%, 80%, 90% or 100%.

In some embodiments, ungulate-derived polyclonal human immunoglobulin compositions are less potent in a complement-dependent cytotoxicity (CDC) assay than a reference product (e.g., human-derived polyclonal immunoglobulin). In some embodiments, the polyclonal immunoglobulins of the disclosure are about 5%, about 10%, about 25%, about 50%, about 100%, about 150%, or more, less potent in a complement-dependent cytotoxicity (CDC) assay than a reference product (e.g., human-derived polyclonal anti-Flu immunoglobulin).

In some embodiments, the ungulate-derived polyclonal human immunoglobulin compositions of the disclosure generate lower toxicity towards CD8+ cells than a reference product (e.g., human-derived polyclonal immunoglobulin). In some embodiments, the ungulate-derived polyclonal human immunoglobulin compositions of the disclosure are at most about 5%, at most about 10%, at most about 25%, at most about 50%, at most about 100%, at most about 150%, or at most about 200% more potent in CD8+ cell killing assay than a reference product (e.g., human-derived polyclonal immunoglobulin).

In some embodiments, the ungulate-derived polyclonal human immunoglobulin compositions of the disclosure generate lower rates of CD4+ T cell apoptosis than a reference product (e.g., human-derived polyclonal immunoglobulin). In some embodiments, ungulate-derived polyclonal human immunoglobulin compositions of the disclosure are at least about 5%, at least about 10%, at least about 25%, at least about 50%, at least about 100%, at least about 150%, or at least about 200% less toxic in a CD4+ cell apoptosis assay than a reference product (e.g., human-derived polyclonal immunoglobulin).

In some embodiments, ungulate-derived polyclonal human immunoglobulin compositions better preserve T_{reg} to conventional T cell ratios than a reference product (e.g. human-derived polyclonal immunoglobulin. In some embodiments, ungulate-derived polyclonal human immunoglobulin compositions are at least about 5%, at least about 10%, at least about 25%, at least about 50%, at least about 100%, at least about 150%, or at least about 200% less toxic to T_{reg} cells than a reference product (e.g., human-derived polyclonal immunoglobulin).

In some embodiments a population of fully human immunoglobulins (or substantially human) specifically binds influenza HA protein, or an immunologically similar antigen.

In one embodiment, avidity of a molecular interaction between two molecules can be measured via techniques such as a surface plasmon resonance (SPR) biosensor technique where one molecule is immobilized on the biosensor chip and the other molecule is passed over the immobilized molecule under flow conditions yielding kₒₙ, k_{off} measurements and hence avidity values.

In one embodiment, a population of human immunoglobulins can be said to bind a target polypeptide disclosed herein or a fragment or variant thereof with an avidity of less than or equal to 10⁻¹×1/sec, 10⁻²×1/sec, or 10⁻³×1/sec. In one embodiment, a population of human immunoglobulins can be said to bind a target polypeptide disclosed herein or a fragment or variant thereof with an avidity less than or equal to 10⁻⁴×1/sec, 10⁻⁵×1/sec, 10⁻⁶×1/sec, or 10⁻⁷×1/sec.

### Methods of Treatment

In some embodiments, methods for providing an effective amount of ungulate-derived human polyclonal immunoglobulin specific for influenza HA protein to the subject for treatment of influenza are provided. In some embodiments, the ungulate-derived human polyclonal immunoglobulin compositions can be adjusted to correspond to the expected seasonal variants of that year by selecting immunoglobulins produced by immunizing transgenic ungulates with two or more influenza strains that match the expected seasonal variants. As used herein, a “therapeutically effective amount” or “effective amount” of the ungulate-derived human polyclonal immunoglobulins is a predetermined amount which confers a therapeutic effect on the treated subject, at a reasonable benefit/risk ratio applicable to any medical treatment. The therapeutic effect can be objective (i.e., measurable by some test or marker) or subjective (i.e., subject gives an indication of or feels an effect or physician observes a change). Effective amounts of ungulate-derived human polyclonal immunoglobulins can range from about 0.001 mg/kg to about 100 mg/kg, about 0.01 mg/kg to about 10 mg/kg, about 0.05 mg/Kg to about 1 mg/kg. The effect contemplated herein includes both medical, therapeutic, and/or prophylactic treatment, as appropriate. The dose of ungulate-derived human polyclonal immunoglobulins administered according to this disclosure to obtain therapeutic and/or prophylactic effects can be determined by the particular circumstances surrounding the case, including, for example, the route of administration, the age, body weight, general health, sex and diet of the patient, the time of administration, route of administration, and rate of excretion of the compositions and the duration of the treatment. The effective amount administered can be determined by the physician in the light of the foregoing relevant circumstances and the exercise of sound medical judgment. The term “effective amount” is intended to also include an effective amount of ungulate-derived human polyclonal immunoglobulins that will bring about a biologically meaningful decrease in the amount of or extent of virus replication or pathogenesis and or decrease in length of illness (fever, joint pains, discomfort) in a subject, or a reduction in loss of body weight in an infected individual. A therapeutically effective amount of ungulate-derived human polyclonal immunoglobulins can be an amount sufficient to reduce or prevent virus load, virus replication, virus transmission, or other feature of pathology such as for example, fever or increased white cell count.

In some embodiments, the ungulate-derived polyclonal human immunoglobulin compositions can improve one or more symptoms of influenza infection. In some embodiments, ungulate-derived polyclonal human immunoglobulin compositions can prevent or decrease lower and/or upper respiratory symptoms. The symptoms that can improved by the compositions described herein include, but are not limited to, fever (over 100° F. or more), chills, fatigue/weakness, chest discomfort, coughing, sneezing, sore throat, runny nose, stuffy nose, throat swelling, skin rash, joint ache, pain around the eyes, muscle aches, vomiting, diarrhea, body aches, and/or headaches.

In some embodiments, the ungulate-derived polyclonal human immunoglobulin compositions can reduce or prevent complications associated with influenza infection. Non-limiting examples of complications include sinus infections, ear infections, pneumonia, inflammation of the heart (myocarditis), brain (encephalitis) or muscle tissues (myositis, rhabdomyolysis), and multi-organ failure (for example, respiratory and kidney failure). In some embodiments, the ungulate-derived polyclonal human immunoglobulin compositions can prevent the occurrence of extreme inflammatory responses in the body, and/or sepsis.

In some embodiments, the ungulate-derived polyclonal human immunoglobulin compositions can result in the reduction or amelioration the severity of an influenza virus infection, an influenza virus disease or a symptom associated therewith. The severity of the infection, disease or symptom can be reduced by about 5%, 10%, 15%, 20%, 25%, 30%, 35%, 40%, 45%, 50%, 55%, 60%, 65%, 70%, 75%, 80%, 85%, 90%, 9%, 100%, about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more.

In some embodiments, the ungulate-derived polyclonal human immunoglobulin compositions can be used to reduce the duration of an influenza virus infection, an influenza virus disease or a symptom associated therewith. The compositions of the disclosure can reduce the duration of infection, disease, or symptoms by about 12 hours, 1 day, 1.5 days, 2 days, 2.5 days, 3 days, 3.5 days, 4 days, 4.5 days, 5 days, 5.5 days, 6 days, 6.5 days, 7 days, 8 days, 9 days, 10 days, 11 days, 12 days, 13 days, 14 days or more.

In some embodiments, the ungulate-derived polyclonal human immunoglobulin compositions can prevent the progression of an influenza virus infection, an influenza virus disease or a symptom associated therewith.

In some embodiments, the ungulate-derived polyclonal human immunoglobulin compositions can prevent the development or onset of an influenza virus infection, an influenza virus disease or a symptom associated therewith.

In some embodiments, the ungulate-derived polyclonal human immunoglobulin compositions can prevent the recurrence of an influenza virus infection, an influenza virus disease or a symptom associated therewith.

In some embodiments, the ungulate-derived polyclonal human immunoglobulin compositions can prevent or reduce the spread/transmission of an influenza virus from one subject to another subject. The compositions can reduce the spread of the influenza virus by about 5%, 10%, 15%, 20%, 25%, 30%, 35%, 40%, 45%, 50%, 55%, 60%, 65%, 70%, 75%, 80%, 85%, 90%, 95%, 100%, about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more.

In some embodiments, the ungulate-derived polyclonal human immunoglobulin compositions can increase the chance of the survival of a subject with an influenza virus infection or a disease associated therewith. The compositions can increase survival by about 5%, 10%, 15%, 20%, 25%, 30%, 35%, 40%, 45%, 50%, 55%, 60%, 65%, 70%, 75%, 80%, 85%, 90%, 95%, 100%, about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more.

In some embodiments, the ungulate-derived polyclonal human immunoglobulin compositions can inhibit or reduce influenza virus replication. The compositions can reduce influenza virus replication by about 5%, 10%, 15%, 20%, 25%, 30%, 35%, 40%, 45%, 50%, 55%, 60%, 65%, 70%, 75%, 80%, 85%, 90%, 95%, 100%, about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more.

In some embodiments, the ungulate-derived polyclonal human immunoglobulin compositions can inhibit or reduce viral load or viral titer of influenza. In one embodiment, the compositions can reduce nasopharyngeal viral load of influenza. The compositions can reduce viral load by about 5%, 10%, 15%, 20%, 25%, 30%, 35%, 40%, 45%, 50%, 55%, 60%, 65%, 70%, 75%, 80%, 85%, 90%, 95%, 100%, about 1-10%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95% or more.

In some embodiments, the ungulate-derived polyclonal human immunoglobulin compositions can prevent influenza infection or disease associated with influenza infection. The term ‘preventing’ or ‘prevention’ refers to a reduction in risk of acquiring or developing a disease or disorder (i.e., causing at least one of the clinical symptoms of the disease not to develop) in a subject that can be exposed to a disease-causing agent, or predisposed to the disease in advance of disease onset.

The ungulate-derived polyclonal human immunoglobulin compositions can be administered to a subject at a dose of about 0.1 mg/kg to 500 mg/kg body weight of the subject. For example, the dose of the composition can be about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 250 mg/kg, 300 mg/kg, 450 mg/kg, 500 mg/kg, about 0.1-1 mg/kg, 0.5-5 mg/kg, 1-10 mg/kg, 5-15 mg/kg, 10-20 mg/kg, 15-25 mg/kg, 20-30 mg/kg, 25-35 mg/kg, 30-40 mg/kg, 35-45 mg/kg, 40-50 mg/kg, 45-55 mg/kg, 50-60 mg/kg, 55-65 mg/kg, 60-70 mg/kg, 65-75 mg/kg, 70-80 mg/kg, 75-85 mg/kg, 80-90 mg/kg, 85-95 mg/kg, 95-100 mg/kg, 10-100 mg/kg, 50-150 mg/kg, 100-200 mg/kg, 150-250 mg/kg, 200-300 mg/kg, 350-450 mg/kg, 300-400 mg/kg, 450-500 mg/kg, 400-500 mg/kg or more. In some embodiments, the compositions can be administered only once or can be administered more than once. When repeated doses are administered, the doses can be administered every hour, every 2 hours, every 6 hours, every 12 hours, every 18 hours, every 24 hours, every 36 hours, every day, every 2 days, every 3 days, every 4 days, every 5 days, every 6 days, every 7 days or more. The repeated doses can be administered at regular intervals. Compositions can be administered by slow infusion, rather than in an intravenous push or bolus. In one example, a higher loading dose can be administered, with subsequent, maintenance doses being administered at a lower level. In another example, a continuous infusion is administered for about five to about ten days.

The compositions can be administered via intravenous, intradermal, subcutaneous, intramuscular, intraperitoneal, intranasal, intranodal and/or intrasplenic route. In one embodiment, the ungulate-derived human polyclonal human immunoglobulins are administered by inhalation as an aerosol.

The ungulate-derived human polyclonal immunoglobulins can be used to treat adults (about 18-65 years of age), elderly adults (about 65 years or more of age), adolescents (about 13-18 years of age), children about (5-13 years of age), toddlers (about 2-5 years of age), babies (about 6 months 2 years of age), infants (about 0-6 months of age), and/or neonate (about 0-1 week of age). In some embodiments, the ungulate-derived human polyclonal immunoglobulins can be used to treat individuals who present one or more symptoms associated with infection by influenza virus. In some embodiments, the ungulate-derived human polyclonal immunoglobulins can be used to treat individuals who have been exposed to the influenza virus but do not or are yet to present one or more symptoms associated with infection by influenza virus. In some embodiments, the ungulate-derived human polyclonal immunoglobulins can be used to treat individuals who test positive in a diagnostic test for influenza virus but are yet to or do not develop one or more symptoms associated with infection by influenza virus.

In some embodiments, administration of an ungulate-derived polyclonal human immunoglobulin composition to a subject may substantially reduce or prevent the development of antibody escape mutations in the influenza virus. An antibody escape mutation refers to one or more genetic changes in a virus that reduces or prevents the binding of the antibody to the virus. In some embodiments, antibody escape mutations are encountered when monoclonal antibodies are used, which can reduce the efficacy of the monoclonal antibody in the subject. Ungulate-derived polyclonal human immunoglobulin compositions, such as those described herein, may be capable of binding to multiple targets on the virus and are therefore can reduce or prevent emergence of antibody escape mutations of the influenza virus. Ungulate-derived polyclonal human immunoglobulin composition.

### Pharmaceutical Compositions

Pharmaceutical compositions comprising an ungulate-derived polyclonal human immunoglobulin composition and one or more pharmaceutically acceptable excipients are provided. In some embodiments, the ungulate-derived polyclonal human immunoglobulin composition specifically binds human influenza HA, or antigenic fragments thereof.

In some embodiments, a pharmaceutical composition comprises at least about 1 mg/mL, at least about 50 mg/mL, at least about 100 mg/mL, or at least about 1,000 mg/mL of ungulate-derived polyclonal human immunoglobulin compositions. In some embodiments, a pharmaceutical composition comprises at least about 100 μg/mL, at least about 250 μg/mL, at least about 500 μg/mL, at least about 750 μg/mL, or at least about 1,000 μg/mL of fully human or substantially human immunoglobulin.

In some embodiments, a fully human or substantially human immunoglobulin is produced in an ungulate. In some embodiments, the ungulate is a bovine.

In some embodiments, the pharmaceutical composition comprises at least 5% fully human immunoglobulin by mass of total immunoglobulin in the pharmaceutical composition.

In some embodiments, the pharmaceutical composition comprises 2% to 5% fully human immunoglobulin by mass of total immunoglobulin in the pharmaceutical composition.

In some embodiments, the pharmaceutical composition can include one or more pharmaceutically acceptable excipients. As used herein, the term “pharmaceutically acceptable excipient” refers to any ingredient, other than active agents (e.g., ungulate-derived polyclonal human immunoglobulins) that are substantially nontoxic and non-inflammatory in a subject. Pharmaceutically acceptable excipients can be biologically or pharmacologically compatible for in vivo use in animals or humans and can be approved by a regulatory agency of the Federal or a state government or listed in the U.S. Pharmacopeia or other generally recognized pharmacopeia for use in animals, and more particularly in humans. Pharmaceutically acceptable excipients can include, but are not limited to, solvents, dispersion media, diluents, inert diluents, buffering agents, lubricating agents, oils, liquid vehicles, dispersion or suspension aids, surface active agents, isotonic agents, thickening or emulsifying agents, preservatives, and the like, as suited to the particular dosage form desired.

In some embodiments, the pharmaceutically acceptable excipient can be a buffer. The buffer can be aa glutamate, an acetate, a histidine, a succinate, or phosphate buffer. The buffer can be at a concentration of about 1 mM to about 50 mM, for example, about 1 mM to about 20 mM, such as about 10 mM. For example, the composition can contain a glutamate buffer at a concentration of about 1 mM to about 20 mM, for example, about 5 mM to about 15 mM, such as about 10 mM. In some embodiments, the glutamate buffer can be glutamic acid monosodium salt.

In some embodiments, the pharmaceutical composition further comprises an excipient, such as sorbitol, sucrose, trehalose, or mannitol. The pharmaceutical composition can include an excipient at a concentration of about 100 mM to about 300 mM, for example, 110 mM to about 270 mM, about 120 mM to about 230 mM, or about 130 mM to about 210 mM, about 170 mM to about 200 mM, or about 180 mM to about 200 mM. For example, the pharmaceutical composition can contain sorbitol at a concentration of about 180 mM to about 300 mM, for example, about 200 mM to about 300 mM, about 200 mM to about 240 mM, about 230 mM to about 270 mM, or about 240 mM to about 260 mM. In some embodiments, the sorbitol can be D-sorbitol. In some embodiments, the pharmaceutical compositions can include 262 mM of D-sorbitol.

In another embodiment, the pharmaceutical composition can include a surfactant, such as a polysorbate, for example, polysorbate 80 (e.g., TWEEN® 80) or polysorbate 20 (e.g., TWEEN® 20). In one embodiment, the concentration of surfactant is about 0.001 mg/mL to about 0.5 mg/mL, about 0.001 mg/mL to about 0.1 mg/mL, for example, about 0.005 mg/mL to about 0.05 mg/mL. As a non-limiting example, the concentration of the surfactant is about 0.05 mg/mL. As used herein, a “surfactant” is a substance that lowers surface tension of a liquid and is used to prevent surface adsorption and act as stabilizers against protein aggregation.

In yet another embodiment, the pharmaceutical composition has a pH of about 4.5 to about 7, for example, pH of about 5 to about 7, pH of about 5 to about 6, pH of about 5.5 to about 7, or pH of about 5.5 to about 6.5. In one embodiment composition has a pH of about 4.5, a pH of about 5, a pH of about 5.5, a pH of about 6, a pH of about 6.5, or a pH of about 7. As a non-limiting example, the pH can be 5.5.

Other suitable excipients can include starch, glucose, lactose, sucrose, gelatin, malt, rice, flour, chalk, silica gel, sodium stearate, glycerol monostearate, talc, sodium chloride, dried skim milk, glycerol, propylene, glycol, water, and/or ethanol.

In some embodiments, pharmaceutical compositions can be a clear, colorless sterile liquid. In some embodiments, pharmaceutical compositions can include glutamic acid monosodium salt, D-sorbitol, and/or TWEEN® 80 (polysorbate). As a non-limiting example, pharmaceutical compositions can include 10 mM glutamic acid monosodium salt, 262 mM D-sorbitol, 0.05 mg/mL TWEEN® 80 (polysorbate). In some embodiments, the pharmaceutical composition can have a pH of about 5.5. In some embodiments, the pharmaceutical composition can be administered to a subject as a liquid solution for injection sodium chloride solution. The liquid solution can contain 0.1%, 0.2%, 0.3%, 0.4%, 0.5%, 0.6%, 0.7%, 0.8%, 0.9%, 1% or more sodium chloride.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

Some embodiments disclosed herein are directed to an ungulate-derived polyclonal human immunoglobulin composition, comprising a population of ungulate-derived polyclonal human immunoglobulins, wherein the population of ungulate-derived polyclonal human immunoglobulins specifically binds an influenza hemagglutinin (HA) protein, wherein the influenza HA protein is two or more of an HA protein of Influenza A and an HA protein of Influenza B and wherein the composition is produced by immunizing a transgenic ungulate with an effective amount of two or more influenza HA proteins from two or more strains of influenza.

In any of the foregoing embodiments, the composition is produced by immunizing the transgenic ungulate with about 0.1 to 10 mg of the two or more influenza HA proteins.

In any of the foregoing embodiments, the two or more influenza HA proteins comprises a full-length influenza HA1 protein and a full-length influenza HA2 protein.

In any of the foregoing embodiments, the population of ungulate-derived polyclonal human immunoglobulins can block influenza HA protein from binding to sialic acid.

In any of the foregoing embodiments, the population of ungulate-derived polyclonal human immunoglobulins has an HAI titer of at least 64.

In any of the foregoing embodiments, the population of ungulate-derived polyclonal human immunoglobulins has an HAI titer of at least 512.

In any of the foregoing embodiments, the population of ungulate-derived polyclonal human immunoglobulins can block one or more of Influenza A virus and Influenza B virus from infecting a mammalian cell.

In any of the foregoing embodiments, the population of ungulate-derived polyclonal human immunoglobulins has a neutralizing concentration of at least 0.01 μg/ml, at least 0.1 μg/ml, or at least 1.0 μg/ml.

In any of the foregoing embodiments, the population of ungulate-derived polyclonal human immunoglobulins has a neutralizing concentration of 0.01 μg/ml to 0.1 μg/ml, or 0.1 μg/ml to 1.0 μg/ml.

In any of the foregoing embodiments, the population of ungulate-derived polyclonal human immunoglobulins has an avidity for influenza HA protein of at least 0.1 1/sec, at least 0.01 1/sec, at least 0.001 1/sec at least 0.0001 1/sec, or at least 0.00001 1/sec.

In any of the foregoing embodiments, wherein the population of ungulate-derived polyclonal human immunoglobulins has an avidity for influenza HA protein of 0.1 to 0.01 1/sec, 0.01 to 0.001 1/sec, 0.001 to 0.0001 1/sec, or 0.0001 to 0.00001 1/sec.

In any of the foregoing embodiments, the population of ungulate-derived polyclonal human immunoglobulins has avidity for influenza HA protein of two or more strains of influenza.

In any of the foregoing embodiments, the composition includes glycans covalently linked to the population of ungulate-derived polyclonal human immunoglobulins, wherein the glycans are at least about 70% N-Glycolylneuraminic acid (NGNA) glycans.

In any of the foregoing embodiments, the composition includes glycans covalently linked to the population of ungulate-derived polyclonal human immunoglobulins, wherein the glycans comprise at least about 90% N-Glycolylneuraminic acid (NGNA) glycans.

In any of the foregoing embodiments, the composition includes glycans covalently linked to the population of ungulate-derived polyclonal human immunoglobulins, wherein the population of human immunoglobulins comprise less than about 50% N-Acetylneuraminic acid (NANA) glycans.

In any of the foregoing embodiments, the composition includes glycans covalently linked to the population of ungulate-derived polyclonal human immunoglobulins, wherein the population of ungulate-derived polyclonal human immunoglobulins comprise less than about 20% N-Acetylneuraminic acid (NANA) glycans.

In any of the foregoing embodiments, the population of ungulate-derived polyclonal human immunoglobulins comprise less than 5% chimeric IgG immunoglobulins.

In any of the foregoing embodiments, the population of ungulate-derived polyclonal human immunoglobulins comprise less than 5% chimeric IgM immunoglobulins.

In any of the foregoing embodiments, the population of ungulate-derived polyclonal human immunoglobulins comprise more than 0.01% of chimeric IgG or chimeric IgM immunoglobulins.

In any of the foregoing embodiments, the population of ungulate-derived polyclonal human immunoglobulins comprise at least about 70% of IgG1.

In any of the foregoing embodiments, the population of ungulate-derived polyclonal human immunoglobulins comprise about 90% IgG1.

In any of the foregoing embodiments, the population of ungulate-derived polyclonal human immunoglobulins comprise less than about 30% IgG2.

In any of the foregoing embodiments, the population of ungulate-derived polyclonal human immunoglobulins comprise about 10% IgG2.

In any of the foregoing embodiments, the population of ungulate-derived polyclonal immunoglobulins comprise less than 4% of one or more of IgG3 and IgG4.

Some embodiments disclosed herein are directed to a method of making an ungulate-derived polyclonal human immunoglobulin composition specific for influenza hemagglutinin (HA) protein, comprising administering an effective amount of at least one influenza HA protein from at least two or more strains of influenza, or a polynucleotide encoding at least one influenza HA protein from at least two or more strains of influenza, to one or more transgenic ungulates, wherein the one or more transgenic ungulates comprises a genome comprising a human immunoglobulin locus or an artificial chromosome comprising a human immunoglobulin locus, and purifying a population of ungulate-derived polyclonal human immunoglobulins from serum or plasma of the one or more transgenic ungulates and combining the ungulate-derived polyclonal human immunoglobulins purified from the one or more transgenic ungulates; wherein the ungulate-derived polyclonal human immunoglobulin composition is made.

In any of the foregoing embodiments, the method includes administering the influenza HA protein 3, 4, 5, or more times.

In any of the foregoing embodiments, the influenza HA protein comprises one or more of a full-length influenza HA1 protein and a full-length influenza HA2 protein.

In any of the foregoing embodiments, the population of ungulate-derived polyclonal human immunoglobulins can block influenza HA protein from binding to sialic acid.

In any of the foregoing embodiments, the population of ungulate-derived polyclonal human immunoglobulins can block Influenza A virus and/or Influenza B virus from infecting a mammalian cell.

In any of the foregoing embodiments, the population of ungulate-derived polyclonal human immunoglobulin can increase survival after Influenza A and/or Influenza B infection.

In any of the foregoing embodiments, the population of human immunoglobulin can improve one or more symptom of Influenza A or Influenza B infection in a subject.

In any of the foregoing embodiments, the population of ungulate-derived polyclonal human immunoglobulins can decrease viral titer in vivo.

In any of the foregoing embodiments, the population of ungulate-derived polyclonal human immunoglobulins has a neutralizing concentration of at least 0.01 μg/ml, at least 0.1 μg/ml, or at least 1.0 μg/ml.

In any of the foregoing embodiments, the population of ungulate-derived polyclonal human immunoglobulins has a neutralizing concentration of 0.01 μg/ml to 0.1 μg/ml, or 0.1 μg/ml to 1.0 μg/ml.

In any of the foregoing embodiments, the population of ungulate-derived polyclonal human immunoglobulins has an avidity for influenza HA protein of at least 0.1 1/sec, at least 0.01 1/sec, at least 0.001 1/sec at least 0.0001 1/sec, or at least 0.00001 1/sec.

In any of the foregoing embodiments, the population of ungulate-derived polyclonal human immunoglobulins has an avidity for influenza HA protein of 0.1 to 0.01 1/sec, 0.01 to 0.001 1/sec, 0.001 to 0.0001 1/sec, or 0.0001 to 0.00001 1/sec.

In any of the foregoing embodiments, the population of ungulate-derived polyclonal human immunoglobulins has an avidity for influenza HA protein of two or more strains of influenza.

In any of the foregoing embodiments, the method includes administering the transgenic ungulate with about 0.1 to 10 mg of the influenza HA protein.

In any of the foregoing embodiments, an excipient is administered with the influenza HA protein.

In any of the foregoing embodiments, the excipient is sodium chloride, monobasic sodium phosphate, dibasic sodium phosphate and/or polysorbate.

Some embodiments disclosed herein are directed to an ungulate-derived polyclonal human immunoglobulin composition specific for influenza HA protein, prepared by the process of administering an effective amount of at least one influenza HA protein from at least two or more strains of influenza, or a polynucleotide encoding influenza HA protein from at least two or more strains of influenza, to one or more transgenic ungulates, wherein the one or more transgenic ungulates comprises a genome comprising a human immunoglobulin locus or an artificial chromosome comprising a human immunoglobulin locus, and purifying a population of ungulate-derived polyclonal human immunoglobulins from serum or plasma of the one or more transgenic ungulates; combining the ungulate-derived polyclonal human immunoglobulins purified from the one or more transgenic ungulates, wherein the ungulate-derived polyclonal human immunoglobulin composition is made.

In any of the foregoing embodiments, the ungulate-derived polyclonal human immunoglobulin composition is produced by administering a transgenic ungulate with an effective amount of the influenza HA protein.

In any of the foregoing embodiments, the ungulate-derived polyclonal human immunoglobulin composition is produced by administering a transgenic ungulate with about 0.1 to 10 mg of the influenza HA protein.

In any of the foregoing embodiments, the influenza HA protein comprises a full-length influenza HA1 protein and a full-length influenza HA2 protein.

In any of the foregoing embodiments, the population of ungulate-derived polyclonal human immunoglobulins can block influenza HA protein from binding to sialic acid.

In any of the foregoing embodiments, the population of ungulate-derived polyclonal human immunoglobulins has an HAI titer of at least 64.

In any of the foregoing embodiments, the population of ungulate-derived polyclonal human immunoglobulins has an HAI titer of at least 512.

In any of the foregoing embodiments, the population of ungulate-derived polyclonal human immunoglobulins can block influenza A virus and/or Influenza B virus from infecting a mammalian cell.

In any of the foregoing embodiments, the population of ungulate-derived polyclonal human immunoglobulins can increase survival after Influenza A and/or Influenza B infection.

In any of the foregoing embodiments, the population of ungulate-derived polyclonal human immunoglobulins can improve one or more symptoms of Influenza A and/or Influenza B infection.

In any of the foregoing embodiments, the population of ungulate-derived polyclonal human immunoglobulins can decrease viral titer in vivo.

In any of the foregoing embodiments, the population of ungulate-derived polyclonal human immunoglobulins has a neutralizing concentration of at least 0.01 μg/ml, at least 0.1 μg/ml, or at least 1.0 μg/ml.

In any of the foregoing embodiments, the population of ungulate-derived polyclonal human immunoglobulins has a neutralizing concentration of 0.01 μg/ml to 0.1 μg/ml, or 0.1 μg/ml to 1.0 μg/ml.

In any of the foregoing embodiments, the population of ungulate-derived polyclonal human immunoglobulins has an avidity for influenza HA protein of at least 0.1 1/sec, at least 0.01 1/sec, at least 0.001 1/sec at least 0.0001 1/sec, or at least 0.00001 1/sec.

In any of the foregoing embodiments, the population of ungulate-derived polyclonal human immunoglobulins has an avidity for influenza HA protein of 0.1 to 0.01 1/sec, 0.01 to 0.001 1/sec, 0.001 to 0.0001 1/sec, or 0.0001 to 0.00001 1/sec.

In any of the foregoing embodiments, the population of ungulate-derived polyclonal human immunoglobulins has avidity for influenza HA protein from two or more strains of influenza.

In any of the foregoing embodiments, the composition includes glycans covalently linked to the population of ungulate-derived polyclonal human immunoglobulins, wherein the glycans are at least about 70% N-Glycolylneuraminic acid (NGNA) glycans.

In any of the foregoing embodiments, the composition includes glycans covalently linked to the population of ungulate-derived polyclonal human immunoglobins, wherein the glycans comprise at least about 90% N-Glycolylneuraminic acid (NGNA) glycans.

In any of the foregoing embodiments, the population of ungulate-derived polyclonal human immunoglobulins comprise less than about 50% N-Acetylneuraminic acid (NANA) glycans.

In any of the foregoing embodiments, the population of ungulate-derived polyclonal human immunoglobulins comprise less than about 20% N-Acetylneuraminic acid (NANA) glycans.

In any of the foregoing embodiments, the population of ungulate-derived polyclonal human immunoglobulins comprise less than 5% chimeric IgG immunoglobulins.

In any of the foregoing embodiments, the population of ungulate-derived polyclonal human immunoglobulins comprise less than 5% chimeric IgM immunoglobulins.

In any of the foregoing embodiments, the population of ungulate-derived polyclonal human immunoglobulins comprise more than 0.01% of chimeric IgG or chimeric IgM immunoglobulins.

In any of the foregoing embodiments, the population of human immunoglobulins comprise at least about 70% of IgG1.

In any of the foregoing embodiments, the population of ungulate-derived polyclonal human immunoglobulins comprise about 90% IgG1.

In any of the foregoing embodiments, the population of ungulate-derived polyclonal human immunoglobulins comprise less than 30% IgG2.

In any of the foregoing embodiments, the population of ungulate-derived polyclonal human immunoglobulins comprise about 10% IgG2.

In any of the foregoing embodiments, the population of ungulate-derived polyclonal immunoglobulins comprise less than 4% of one or more of IgG3 and IgG4.

Some embodiments disclosed herein are directed to a pharmaceutical composition, comprising the composition of any of the foregoing embodiments and optionally one or more pharmaceutically acceptable excipients.

In any of the foregoing embodiments, the pharmaceutical composition includes optionally one or more pharmaceutically acceptable excipients.

In any of the foregoing embodiments, one or more pharmaceutically acceptable excipients are glutamic acid monosodium salt, D-sorbitol, and/or polysorbate.

In any of the foregoing embodiments, the composition is at a pH of about 5-6.

In any of the foregoing embodiments, the pharmaceutical composition is a liquid solution in sodium chloride.

Some embodiments disclosed herein are directed to a method of treating infection with influenza virus in a subject in need thereof, comprising administering an effective amount of the composition of any of the previous embodiments or the pharmaceutical composition any of the previous embodiments to the subject.

The following are provided for exemplification purposes only and are not intended to limit the scope of the embodiments described in broad terms above.

### EXAMPLES

The following specific examples are to be construed as merely illustrative, and not limitative of the remainder of the disclosure in any way whatsoever.

### Example 1

Generation of SAB-176 hIgG

SAB-176 is a purified polyclonal human immunoglobulin G (hIgG) designed to specifically bind to Type A and Type B influenza viruses. The product can be used as a therapeutic agent to treat patients who are infected with Type A and Type B influenza viruses. Tc bovines were hyperimmunized with a quadrivalent seasonal influenza recombinant HAG (rHA0) protein vaccine at 0.2 to 0.9 milligrams (mg) rHA0 per strain via intramuscular injections. The quadrivalent rHA0 proteins from influenza A (H1N1 and H3N2) and Influenza B (B/Victoria lineage and B/Yamagata lineage) were produced and purified from insect cells. HAG is a single precursor polypeptide, which is generally cleaved into two polypeptides (HA1 and HA2). The hIgG is purified from the plasma of Tc Bovines hyperimmunized a minimum of five times. SAB-176 has an IgG1 subclass content of approximately 80-90%. In contrast, only approximately 60% of human derived IVIg is subclass IgG1. IgG1 strongly activates complement and effector cells (natural killer cells, neutrophils, monocytes, etc.) of the innate immune system. Each IgG molecule comprises two heavy chains and two light chains linked with disulfide bonds. The entire IgG molecule has a molecular weight of approximately 150 kilodaltons as evidenced by size-exclusion high performance liquid chromatography (SEC HPLC) analysis. Each heavy chain has a molecular weight of approximately 50 kilodaltons, and each light chain has a molecular weight of approximately 25 kilodaltons as measured by sodium dodecyl sulfate polyacrylamide gel electrophoresis (SDS PAGE) under reducing conditions.

SAB-176 contains less than 2% of chimeric IgG, which contains human IgG heavy chain and bovine light chain. Other impurities in SAB-176 include bovine plasma proteins, such as bovine serum albumin (BSA), and bovine IgG, each of which are below a level of 100 parts per million (ppm). It is expected that the IgG antibodies in SAB-176 will have a 28 day half-life and distribution in humans similar to that of SAB-301, an anti-Middle East Respiratory Syndrome Coronavirus [MERS-CoV] Tc bovine hIgG, as previously evaluated in a Phase 1 clinical trial and typical for human IgG antibodies.

SAB-176 is a clear, colorless sterile liquid for parenteral use (e.g., injection such as intravenous, subcutaneous, intramuscular) formulated in 10 mM glutamic acid monosodium salt, 262 mM D-sorbitol, 0.05 mg/mL TWEEN® 80 (polysorbate), pH 5.5 and is stored at 2-8° C. Once the drug product is removed from 2-8° C. and diluted into the saline intravenous (IV) bag, the drug product is stable for 24 hours at room temperature.

SAB-176 is highly purified from the plasma of Tc bovines that were immunized with a quadrivalent recombinant influenza HA protein vaccine produced and purified from insect cells. Upon receipt of acceptable release tests, the plasma is then thawed, pooled, fractionated by CA, and clarified by depth filtration in the presence of filter aid. The clarified sample containing hIgG is further purified by affinity chromatography, first using an anti-human IgG affinity column to bind hIgG and remove Bovine Plasma Protein (BPP) followed by a low pH treatment, and second, by passing through an anti-bovine IgG heavy chain (HC) specific affinity column to further remove residual IgG molecules that contain a bovine HC. The Tc bovine-derived hIgG fraction is then concentrated and diafiltered prior to a Q Sepharose chromatography polishing step, nanofiltration, final buffer exchange, concentration and sterile filtration to provide the Drug Substance. Released Drug Substance is passed through a terminal sterile filter and filled into glass vials.

### Example 2: Potency of SAB-176 by Hemagglutination Inhibition Assay (HAI) and Microneutralization (MN)

SAB-176 was evaluated in vitro for potency using HAI and MN assays and has shown potency to the strains used to produce SAB-176, as well as unimmunized strains of influenza. SAB-176 was also evaluated for potential cross reactivity in a GLP tissue cross-reactivity study. No staining was present with SAB-176 in the human panel examined. Results for these studies are presented below. The potency of SAB-176 was evaluated by HAI and MN assays.

The HAI assay is a primary method for determining the potency of antibodies against influenza virus. The assay relies on the ability of the HA protein on the surface of influenza virus to bind to sialic acids on the surface of red blood cells (RBCs). Specific attachment of antibody to the antigenic sites on the HA molecule interferes with the binding between the viral HA and sialic acids on the RBCs and inhibits the agglutination, which would otherwise occur between the direct interaction of the virus and the RBCs.

Test articles were diluted to 5 mg/ml and serially diluted 2-fold in 96-V-well microtiter plates. The working stock of the influenza virus strain of interest was standardized to an HA titer of eight and added to each well. After incubation, standardized chicken (or turkey if H3N2 viral strains were to be evaluated) RBC solution was added to each well on the plate containing virus and diluted test articles. The assay plates were then incubated until the control wells containing virus and no antibody demonstrated complete hemagglutination. This occurred when the RBCs in the buffer control sample form a distinct button at the bottom of the well. The HAI titer was defined as the reciprocal of the highest dilution of antibody test article that inhibited RBC hemagglutination by the selected influenza virus.

The MN assay is a standard technique for measuring the infectivity of the influenza virus and the inhibition of virus replication. Unlike typical plaque reduction assays that rely on visible plaques, this assay is based on the ability of anti-influenza antibodies to prevent infection of Madin-Darby Canine Kidney (MDCK) in vitro, and as such, represents a more mechanistically relevant estimation of antibody-mediated protection compared to HAI alone. The protocol utilizes quantitative titration to define the amount of input neutralizing antibodies required to effectively neutralize the influenza virus from infecting MDCK cells. Virus infectivity is quantified by measuring the relative amount of virus nucleoprotein (NP) present in treated vs non-treated MDCK cells.

The initial material of SAB-176 was evaluated for HAI and microneutralization titers against the influenza strains from which their HA was derived for hyperimmunization of the Tc Bovines, as well as influenza strains from which their HA was not included. HAI and MN assays were conducted as a part of Drug Product release testing for all lots.

Table 2 and Table 3 below show HAI and MN titers of the of SAB-176 compared to anti-Flu hIVIg used in a pilot study entitled, “An Anti-Influenza Virus Hyperimmune Intravenous Immunoglobulin Pilot Study”, and a negative control IgG. In Table 2, α-Flu is abbreviation for anti-Flu.

TABLE 2
Hemagglutination Inhibition (HAI) Titers

| | | A/Michigan/ | | A/Singapore/ | | | B/Maryland/ | | B-Phuket/ |
|---|---|---|---|---|---|---|---|---|---|
| | | 45/2015 | | INFIMH- | | | 15/2016 | | 3073/2013 |
| Purified | A/California/ | pH1N1 | A/Hong Kong/ | 16-0019/2016 | A/Switzerland/ | B/Brisbane/ | B-Vic | B/Colorado/ | B-Yam |
| Sample | 4/2009 | (vaccine | 4801/2014 | H3N2 (vaccine | 9715293/2013 | 60/2008 | (vaccine | 06/2017 | (vaccine |
| (5 mg/ml) | pH1N1 | strain) | H3N2 | strain) | H3N2 | B-Vic | strain) | B-Vic | strain) |
| SAB-176 | >2048 | >2048 | 1024 | 512 | >2048 | 64 | 512 | 512 | 512 |
| α-Flu | 32 | 32 | 64 | 32 | 128 | 8 | 8 | 8 | 16 |
| human | | | | | | | | | |
| IVIg | | | | | | | | | |
| Negative | <1 | <1 | <1 | <1 | <1 | <1 | <1 | <1 | <1 |
| Control | | | | | | | | | |

TABLE 3
Microneutralization (MN) Titers

| | | A/Michigan/ | | A/Singapore/ | | | B/Maryland/ | | B-Phuket/ |
|---|---|---|---|---|---|---|---|---|---|
| | | 45/2015 | | INFIMH- | | | 15/2016 | | 3073/2013 |
| Purified | A/California/ | pH1N1 | A/Hong Kong/ | 16-0019/2016 | A/Switzerland/ | B/Brisbane/ | B-Vic | B/Colorado/ | B-Yam |
| Sample | 4/2009 | (vaccine | 4801/2014 | H3N2 (vaccine | 9715293/2013 | 60/2008 | (vaccine | 06/2017 | (vaccine |
| (5 mg/ml) | pH1N1 | strain) | H3N2 | strain) | H3N2 | B-Vic | strain) | B-Vic | strain) |
| SAB-176 | >1024 | 512 | >1024 | >1024 | 512 | 128 | >1024 | 128 | 512 |
| α-Flu | 256 | 8 | 64 | 64 | 32 | 8 | 32 | <4 | 32 |
| human | | | | | | | | | |
| IVIg | | | | | | | | | |
| Negative | <1 | <1 | <1 | <1 | <1 | <1 | <1 | <1 | <1 |
| Control | | | | | | | | | |

Summary: HAI and MN data demonstrates that SAB-176 has high titers against immunized and unimmunized influenza strains.

Table 4, Table 5, and Table 6 show the HAI assay result for individual lots of SAB-176.

TABLE 4
Hemagglutination Inhibition (HAI) Titers for individual SAB-176 lots

| | H1N1 | | | | | | |
|---|---|---|---|---|---|---|---|
| Sample | A/California/ | A/California/ | | | A/Guangdong- | A/Guangdong- | A/Victoria/ |
| Started at | 4/2009 | 4/2009 | A/Michigan/ | A/Brisbane/ | maonan/2019 | maonan/2019 | 2570/2019 |
| 5 mg/ml | (hVIVO) | (Huber stock) | 45/2015 | 02/2018 | (Egg) | (Cell) | (2021-22) |
| SAB-176 Lot 4 | 1:512 Λ | 1:512 Λ | 1:512 * | 1:512 * | 1:512 * | 1:512 * | 1:256 Λ |
| V3-V12 | (8X) | (8-16X) | (16X) | (16-32X) | (16-32X) | (16X) | (16-32X) |
| SAB-176 Lot 3 | 1:512 Λ | 1:512 Λ | 1:512 * | 1:512 * | 1:512 * | 1:512 * | 1:256 Λ |
| V3-V12 | (8X) | (8-16X) | (16X) | (16-32X) | (16-32X) | (16X) | (16-32X) |
| SAB-176 Lot 2 | 1:1024 Λ | 1:512 Λ | 1:512 * | 1:512 Λ | 1:512 Λ | 1:512 Λ | 1:256 |
| V3-V5 | (16X) | (8-16X) | (16X) | (16-32X) | (16-32X) | (16X) | (16-32X) |
| SAB-176 Lot 1 | 1:512 Λ | 1:512 Λ | 1:512 * | 1:512 Λ | 1:512 Λ | 1:512 Λ | 1:256 Λ |
| V3 | (8X) | (8-16X) | (16X) | (16-32X) | (16-32X) | (16X) | (16-32X) |
| Anti-Flu hIVIG | 1:64 | 1:32 | 1:32 | 1:32 | 1:16 | 1:32 | 1:8 |
| 2013 | | | | | | | |
| Anti-Flu hIVIG | 1:64 | 1:32 | 1:32 | 1:16 | 1:16 | 1:32 | 1:16 |
| 2017 | | | | | | | |
| Anti-Flu hIVIG | 1:64 | 1:64 | 1:32 | 1:32 | 1:32 | 1:32 | 1:16 |
| 2018 | | | | | | | |
| NC Ab | <1 | <1 | <1 | <1 | <1 | <1 | <1 |

Λ Non-Vaccine strains
* Vaccine strains

TABLE 5
Hemagglutination Inhibition (HAI) Titers for individual SAB-176 lots
H₃N₂

| Sample | | A/Singapore/ | | A/Hong Kong/ | A/Cambodia/ |
|---|---|---|---|---|---|
| Started at | | INIFMH-16- | A/Kansas/ | 2671/2019 | e0826360/2020 |
| 5 mg/ml | [symbol] | 0019/2016 | 14/2017 | (Egg) | (2021-22) |
| SAB-176 Lot 4 | | 1:512* | 1:256* | 1:256* | 1:256 ∧ |
| V3-V12 | | (8-32X) | (8-64X) | (16-32X) | (8-16X) |
| SAB-176 Lot 3 | | 1:512* | 1:512* | 1:256* | 1:256 ∧ |
| V3-V12 | | (8-32X) | (16-128X) | (16-32X) | (8-16X) |
| SAB-176 Lot 2 | | 1:512* | 1:64 ∧ | 1:128 ∧ | 1:256 |
| V3-V5 | | (8-32X) | (2-16X) | (8-16X) | (8-16X) |
| SAB-176 Lot 1 | | 1:512* | 1:64 ∧ | 1:64 ∧ | 1:128 ∧ |
| V3 | | (8-32X) | (2-16X) | (4-8X) | (4-8X) |
| Anti-Flu hIVIG | | 1:16 | 1:4 | 1:8 | 1:16 |
| 2013 | | | | | |
| Anti-Flu hIVIG | | 1:64 | 1:32 | 1:16 | 1:32 |
| 2017 | | | | | |
| Anti-Flu hIVIG | | 1:64 | 1:32 | 1:16 | 1:32 |
| 2018 | | | | | |
| NC Ab | | <1 | <1 | <1 | <1 |

∧ Non-Vaccine strains
*Vaccine strains [symbol]
indicates data missing or illegible when filed

TABLE 6
Hemagglutination Inhibition (HAI) Titers for individual SAB-176 lots

| Sample | B-Vic | | | B-Yam | | |
|---|---|---|---|---|---|---|
| Started at | B/Maryland/ | B/Colorado/ | B/Washington/ | B/Phuket/ | B/California/ | |
| 5 mg/ml | 15/2016 | 06/2017 | 02/2019 | 3073/2013 | 12/2015 | G4HA |
| SAB-176 Lot 4 | 1:256 * | 1:256 * | 1:128 * | 1:128 * | 1:128 Λ | 1:512 Λ |
| V3-V12 | (16-32X) | (16-32X) | (16-32X) | (16X) | (16-32X) | (16X) |
| SAB-176 Lot 3 | 1:128 * | 1:256 * | 1:64 * | 1:128 * | 1:128 Λ | 1:512 Λ |
| V3-V12 | (8-16X) | (16-32X) | (8-16X) | (16X) | (16-32X) | (16X) |
| SAB-176 Lot 2 | 1:256 * | 1:256 Λ | 1:128 Λ | 1:256 * | 1:128 Λ | 1:512 Λ |
| V3-V5 | (16-32X) | (16-32X) | (16-32X) | (32X) | (16-32X) | (16X) |
| SAB-176 Lot 1 | 1:256 * | 1:256 Λ | 1:64 Λ | 1:128 * | 1:128 Λ | 1:512 Λ |
| V3 | (16-32X) | (16-32X) | (8-16X) | (16X) | (16-32X) | (16X) |
| Anti-Flu hIVIG | 1:8 | 1:8 | 1:4 | 1:8 | 1:4 | 1:32 |
| 2013 | | | | | | |
| Anti-Flu hIVIG | 1:16 | 1:16 | 1:8 | 1:8 | 1:8 | 1:32 |
| 2017 | | | | | | |
| Anti-Flu hIVIG | 1:16 | 1:16 | 1:8 | 1:8 | 1:8 | 1:32 |
| 2018 | | | | | | |
| NC Ab | <1 | <1 | <1 | <1 | <1 | <1 |

Λ Non-Vaccine strains
* Vaccine strains

As shown in Tables 2-6 SAB-176 was more potent than anti-Flu hIVIg.

### Example 3: Evaluation of SAB-176 for Therapeutic Treatment of an Influenza A/CA/04/2009 (H1N1Pdm) Infection in BALB/c Mice

The efficacy of SAB-176 was evaluated for therapeutic treatment of an influenza A/CA/04/2009 (H1N1pdm) in BALB/c mice. Mice were treated 12 hours post-infection with SAB-176, anti-Flu hIVIg, or control IgG that is not relevant to influenza (also referred to herein as “irrelevant IgG”). Aerosol, intranasal (IN), and intraperitoneal (IP) administrations were evaluated. Only the IP administration data is shown below. Mortality and weight loss were the primary endpoints.

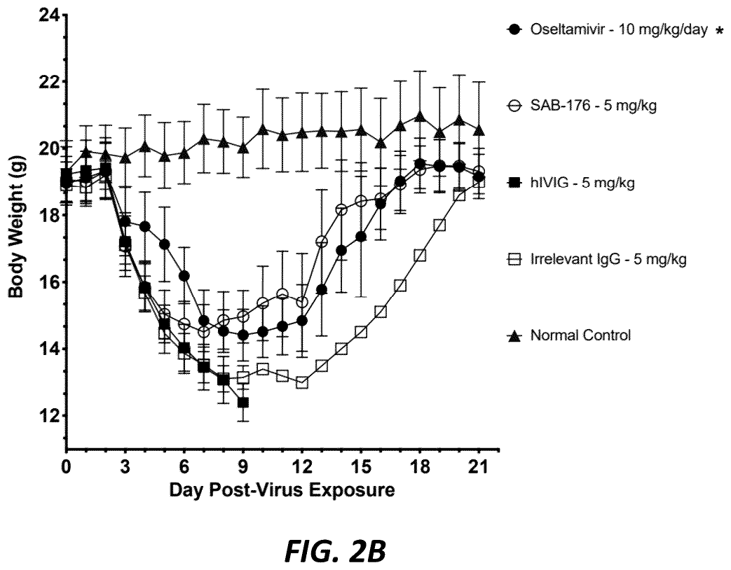

FIG. 2 shows survival curves (Panel A) and mean body weights (Panel B) for mice treated intraperitoneally with SAB-176 or anti-Flu hIVIg. When given via the IP route, SAB-176 at a dose of 5 mg/kg completely protected mice from mortality but did not prevent weight loss. IP administration of anti-Flu hIVIg at a dose of 5 mg/kg did not prevent mortality or weight loss.

### Example 4: Evaluation of SAB-176 for Treatment of an Oseltamivir-Resistant Influenza A/Hong Kong/2369/2009 (H1N1Pdm) Infection in BALB/c Mice

The purpose of this study was to assess the efficacy of SAB-176 by IN or IP administration for treatment of an oseltamivir-resistant influenza A/Hong Kong/2369/2009 (H1N1pdm) virus infection in BALB/c mice; only data from the IP administration study is presented here. The mice were treated 12 hours post-virus exposure with SAB-176, anti-Flu hIVIg, or an irrelevant IgG (placebo). Animals were observed daily for 21 days following treatment for mortality, weight loss and adverse events. Mortality and weight loss were the primary endpoints.

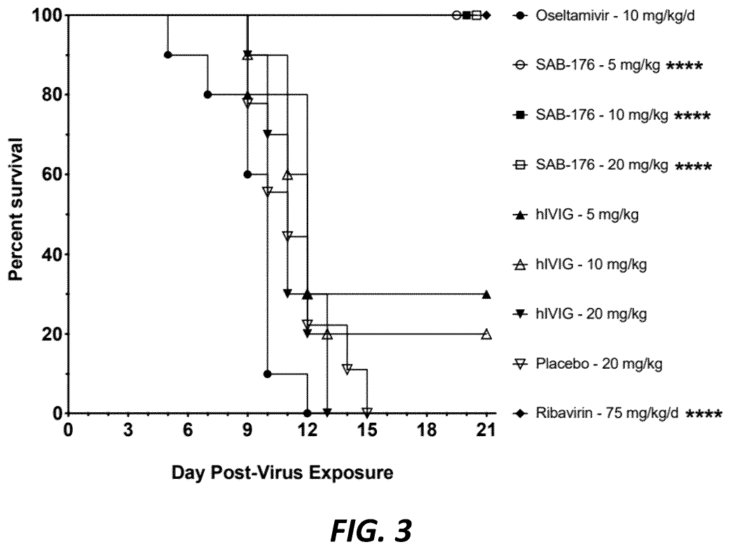

FIG. 3 shows Kaplan-Meier survival curves for mice treated via IP route with SAB-176, anti-Flu hIVIg, or an irrelevant IgG. Mice treated with a single administration of SAB-176 at a dose of 5, 10, or 20 mg/kg completely protected mice from mortality. Three of ten mice (30%) treated with anti-Flu hIVIg at a dose of 5 mg/kg survived the infection. Two of ten mice (20%) treated with anti-Flu hIVIg at a dose of 10 mg/kg survived the infection. None of the ten mice treated with anti-Flu hIVIg at a dose of 20 mg/kg survived the infection. None of the irrelevant IgG (placebo) treated mice survived the infection.

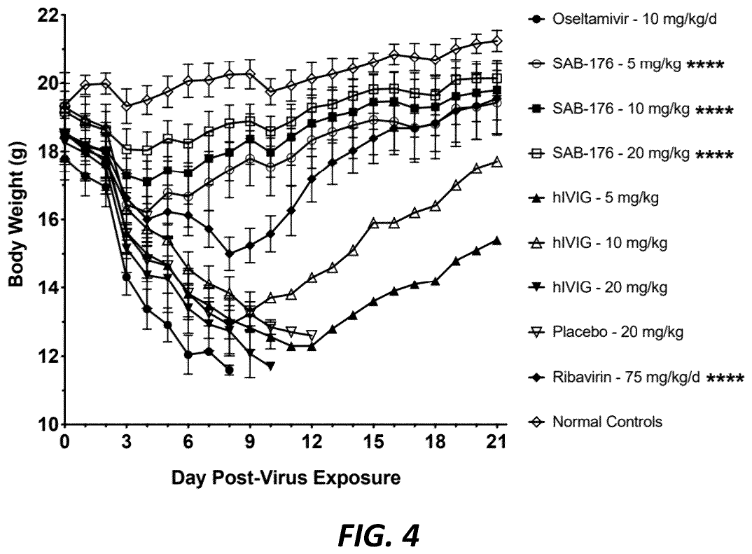

Mean body weights for mice treated IP with SAB-176, anti-Flu hIVIg, or an irrelevant IgG (placebo) are shown in FIG. 4. IP administration of SAB-176 at doses of 5, 10, or 20 mg/kg protected mice from infection-associated weight loss. IP route treatment with anti-Flu hIVIg at doses of 5, 10, or 20 mg/kg did not protect mice from weight loss after infection.

IP administration of SAB-176, but not anti-Flu hIVIg, provided protection from mortality. Only SAB-176, and not anti-Flu hIVIg, was able to protect mice from weight loss when administered by IP route. SAB-176 was highly effective at preventing mortality and weight loss by IP route in mice infected with an influenza A/HK/2369/09 H275Y (H1N1pdm) virus. Neutralization potency of SAB-176 against H1N1pdm is summarized in Table 7 and Table 8.

TABLE 7
SAB-176 in vitro virus neutralization potency
of SAB-176 against H1N1pdm

| | Neutralizing Concentration | |
|---|---|---|
| | A/CA/04/2009 | |
| Antibody | (mouse-adapted) | A/CA/07/2009 |
| SAB-176 | 0.1 μg/mL | 0.1 μg/mL |
| Anti-Flu Human IVIG | 3.9 μg/mL | 1.9 μg/mL |
| Negative Control | >1000 μg/mL | >1000 μg/mL |

TABLE 8
SAB-176 in vitro virus neutralization potency compared to anti-Flu
human IVIG against oseltamivir resistant H1N1pdm viruses

| | Neutralizing Concentration | | |
|---|---|---|---|
| | A/North | | A/Hong |
| | Carolina/ | A/Texas/ | Kong/ |
| | 39/2009 | 23/2012 | 2369/2009 |
| | (H1N1pdm) | (H1N1pdm) | (H1N1pdm) |
| Antibody | H275Y | H275Y | H275Y |
| SAB-176 | 0.78 μg/mL | 0.78 μg/mL | 0.39 μg/mL |
| Anti-Flu Human | 100 μg/mL | 100 μg/mL | 25 μg/mL |
| IVIG | | | |
| Negative Control | >100 μg/mL | >100 μg/mL | >100 μg/mL |

### Example 5: Evaluation of SAB-176 in Ferret Model of Influenza Challenge

The purpose of this study was to demonstrate the therapeutic potential of SAB-176. The objective of this study was limited to the therapeutic delivery of SAB-176 at 50 mg/kg administered intravenously in ferrets that had been challenged with the A/California/4/2009-H1N1 influenza A virus. The ferret response to challenge was monitored daily and reported as a clinical score that factors in sneezing and behavioral activity, as described in Table 9 and Table 10 below. To demonstrate therapeutic efficacy, samples were collected from the upper respiratory tract (nasal wash), the olfactory bulb, the soft palate, and lungs. Serum samples were collected to show proper transfer of SAB-176 into individual ferrets.

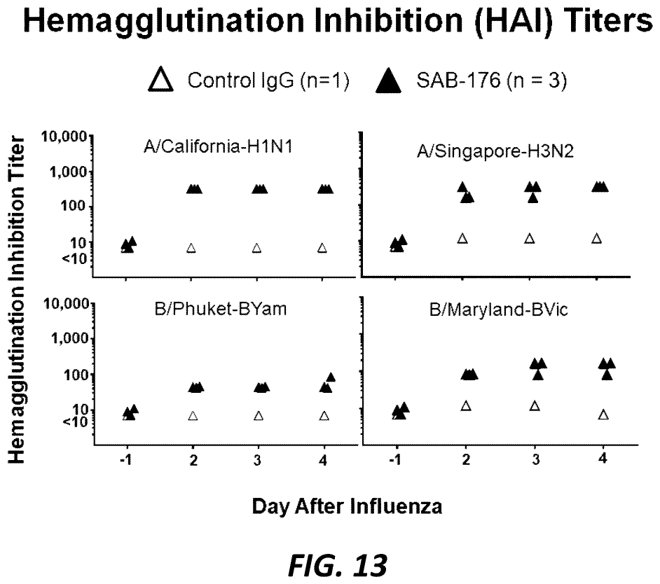

Antibodies against A/California/4/2009-H1N1, A/Singapore/INFIMH-16-0019/2016 (H3N2), B/Phuket/3073/2013-BYam, and B/Maryland/15/2016-Bvic, which match the isolates in the vaccine that were used to create SAB-176, were detected in ferrets using the hemagglutination inhibition assay (FIG. 13).

The protective immunity of SAB-176 against A/California/4/2009-H1N1 was confirmed through direct challenge. Ferrets can be naturally infected with human influenza viruses, and they are a well-accepted model for studying human influenza virus infection. Ferrets infected with influenza virus demonstrate a transient increase in temperature within 24-48 hours post-challenge, they shed virus that can be collected within nasal wash fluid after inoculation, and they demonstrate signs of illness including decreased activity and increased sneezing.

TABLE 9
Ferret Study Experimental Design

| | | | Number | | | | Daily | |
|---|---|---|---|---|---|---|---|---|
| | | Treatment Lot | of | Dose | | Dose | Observations | |
| Group | Challenge | Number | Ferrets | Level | Route | Timepoints | Day −1 to 4 | Sample Collection |
| SAB- | A/California/ | PR1801362FL | 3 | 50 | IV | Day 1, 2, | Sneezing | Nasal wash |
| 176 | 4/2009-H1N1 | | | mg/kg | | and 3 post- | Activity | (Day −1 to Day 4) |
| | | | | | | challenge | Temperature | Olfactory bulb, |
| | | | | | | | Weight | soft palate, and lungs |
| Control | A/California/ | PD1501135NC | 1 | 50 | IV | | | (Day 4) |
| IgG | 4/2009-H1N1 | | | mg/kg | | | | Serum |
| | | | | | | | | (Day −1 and Day 2, 3, 4) |

TABLE 10
Clinical Score for Activity and Sneezing

| Score | 0 | 1 | 2 | 3 |
|---|---|---|---|---|
| Activity | Alert and | Alert and | Alert but | Neither alert |
| | Playful | Playful | not Playful | nor playful |
| | without | with | with | with |
| | Stimulation | Stimulation | Stimulation | Stimulation |
| Sneezing | 0 | 1-10 | >10 | Respiratory |
| | | | | Distress |

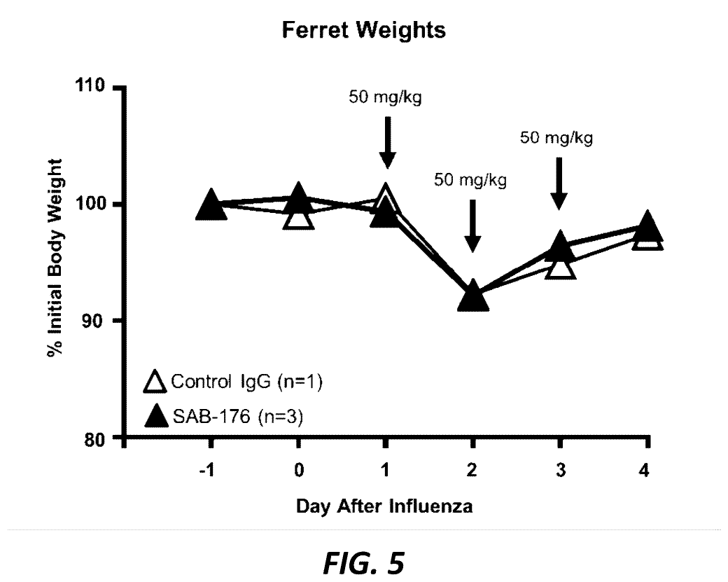

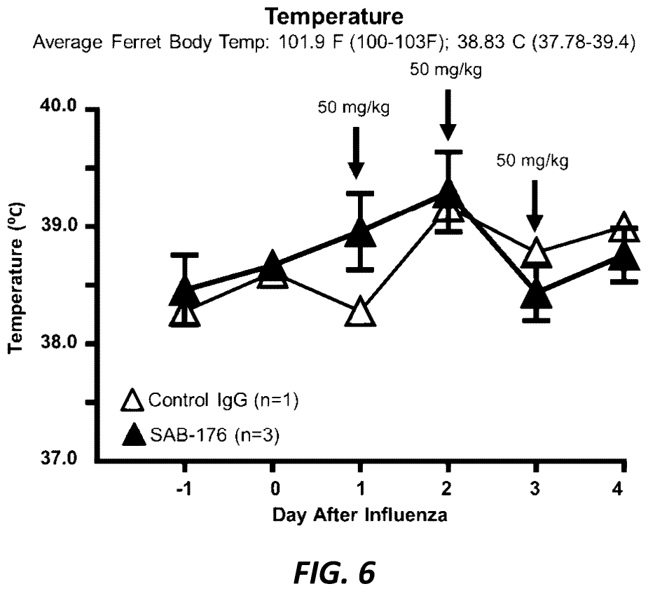

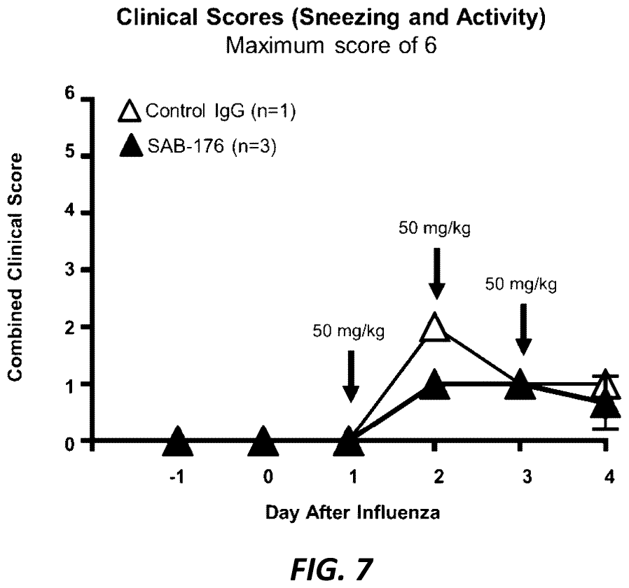

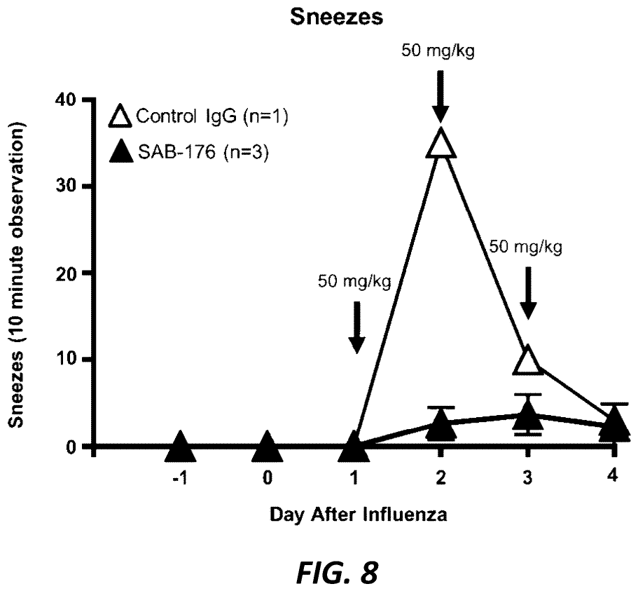

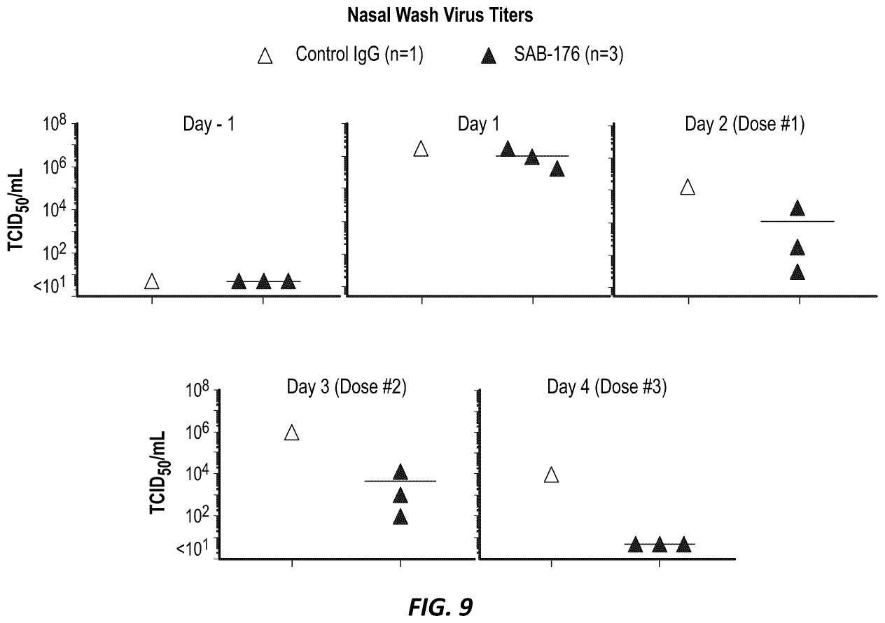

Three ferrets received SAB-176 and one ferret received negative control IgG, as described above. Ferrets that were infected with the A/California/4/2019-H1N1 influenza virus showed both a decrease in percent initial body weight (FIG. 5) and an increase in temperature (FIG. 6). The lowest body weight and highest temperature coincided with 24 hours post-transfer of hIgG and was observed for both SAB-176 and negative control IgG. At the same 48-hour timepoint post-challenge, clinical signs of infection, reported using the combined clinical score as described above, were increased in the ferret that received negative control Tc hIgG (FIG. 7), and this score was driven by a significant increase in sneezing (FIG. 8). As expected, all animals were negative for influenza on day −1 and showed similar levels of influenza virus on day 1 post influenza challenge. However, nasal wash virus titers were decreased in ferrets that received SAB-176 on days 2, 3, and 4 post-influenza when compared to Control IgG (FIG. 9).

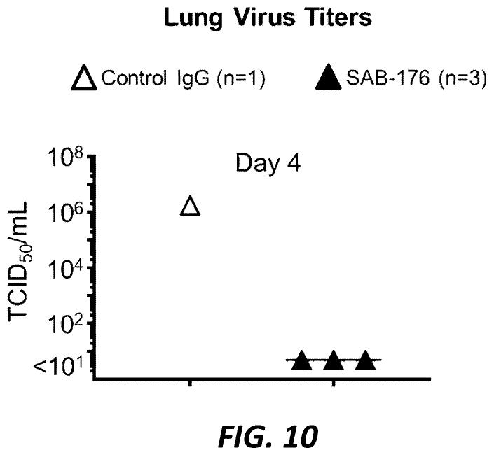

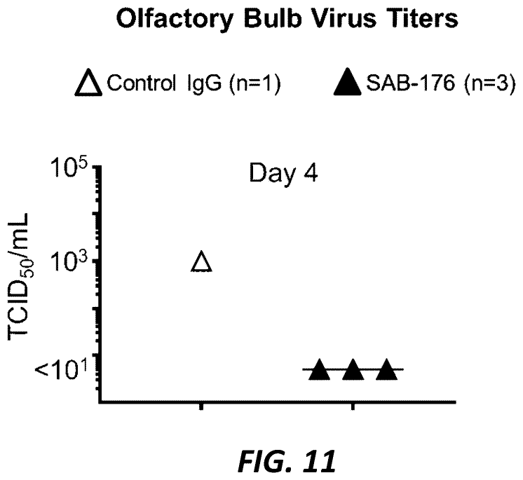

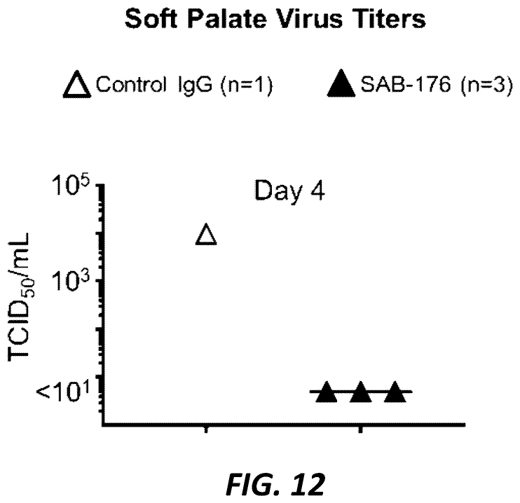

These reductions in viral titers coincided with 24, 48, and 72 hours following the delivery of SAB-176 via intravenous (IV) route. At the conclusion of the study (day 4), influenza virus was detected in the lung (FIG. 10), olfactory bulb (FIG. 11), and soft palate (FIG. 12) of the ferret that received negative control IgG, while virus titers were below the detectable limit for the three ferrets that received SAB-176 IV.

The successful transfer and functional effects of the polyclonal SAB-176 antibodies were confirmed with HAI titers against A/California/4/2009-H1N1, A/Singapore/INFIMH-16-0019/2016 (H3N2), B/Phuket/3073/2013-Byam, and B/Maryland/15/2016-Bvic. Antibodies were detected within 24 hours post-IV transfer (FIG. 13).

The data shows that SAB-176 can limit infection of ferrets challenged with the A/California/4/2009-H1N1 influenza virus. This is supported by the reduction in clinical score seen on Day 2 after influenza virus challenge (24 hours after the first dose of SAB-176). This clinical score reflected both the activity of the ferrets and the sneezing that occurred after inoculation. While all ferrets were shedding virus at approximately the same level on Day 1 post-influenza virus challenge, which was prior to transfer of hIgG, virus titers in the group that received SAB-176 were lower than in the group that received Control IgG on days 2, 3, and 4 post-challenge. This reduction in virus titer in the upper respiratory tract was also seen in the olfactory bulb, soft palate, and the lung.

Finally, as evidence of SAB-176 being transferred into ferrets, the hemagglutination inhibition assay was used to show that serum collected from ferrets interacted with the A/California/7/09 (H1N1), A/Singapore/INFIMH-16-0019/2016 (H3N2), B/Phuket/3073/2013-Byam, and B/Maryland/15/2016-BVic influenza viruses that were included in the vaccine used in the hyperimmunization schedule to produce SAB-176. These antibodies were detected on days 2, 3, and 4 which coincided with 24 hours after delivery of first, second and third doses of SAB-176, respectively.

Overall, these findings show that SAB-176 was effectively delivered to these ferrets, and that it was able to limit the clinical signs of infection and to reduce virus in the upper respiratory tract, the olfactory bulb, the soft palate, and the lungs. Since antibodies against A/Singapore/INFIMH-16-0019/2016 (H3N2), B/Phuket/3073/2013-Byam, and B/Maryland/15/2016-Bvic influenza viruses were detected in the serum samples collected from ferrets that received SAB-176, it is likely that these antibodies could limit infection with these additional influenza viruses as well. IV administration of SAB-176 to ferrets challenged with A/California/4/2009-H1N1 influenza virus showed a reduction in clinical signs of disease, reduced viral titer in nasal wash, and prevention of virus from taking hold in the lungs, olfactory bulb, and soft palate, indicating that SAB-176 has the potential to be an effective IV therapeutic for influenza infection.

### Example 6: A GLP Single Dose Study of SAB-176 Delivered by Intravenous Infusion in Rabbits with a 49 Day Recovery Period

A single dose study in rabbits was conducted to determine the potential toxicity of SAB-176 for influenza treatment. Results from this study indicate that administration of SAB-176 once by IV infusion was well tolerated in rabbits at levels of 362.65 and 725.30 mg/kg/day. The details of this study are described below.

The objectives of this study were to determine the potential toxicity of SAB-176 for the treatment of Type A and Type B influenza illnesses, when given as a single intravenous infusion to rabbits and to evaluate the potential reversibility of any findings. In addition, the toxicokinetic characteristics of SAB-176 were determined.

The study design was as described in Table 11 below. The following parameters and end points were evaluated in this study: clinical signs, body weights, body weight gains, food consumption, ophthalmology, clinical pathology parameters (hematology, coagulation, clinical chemistry, and urinalysis), toxicokinetic parameters, immunogenicity analysis, gross necropsy findings, organ weights, and histopathologic examinations.

TABLE 11
Experimental Design for SAB-176 Single Dose Study in Rabbits-Main and Recovery

| | | | Dose | Dose | No. of Animals | | | |
|---|---|---|---|---|---|---|---|---|
| Group | Test | Dose Level | Volume^{a} | Concentration | Main Study | | Recovery Study | |
| No. | Material | (mg/kg/day) | (mL/kg) | (mg/mL) | Males | Females | Males | Females |
| 1 | Control | 0 | 10 | 0 | 5 | 5 | 3 | 3 |
| | Article | | | | | | | |
| 2 | Test Article | 362.65 | 5 | 72.53 | 5 | 5 | 3 | 3 |
| | (SAB-176) | | | | | | | |
| 3 | Test Article | 725.30 | 10 | 72.53 | 5 | 5 | 3 | 3 |
| | (SAB-176) | | | | | | | |

^{a}Based on the most recent body weight measurement.

There were no test article-related effects noted on clinical signs, body weights, body weight gains, food consumption, ophthalmology, gross necropsy findings, organ weights, or histopathologic examinations.

There were no test article-related adverse effects on clinical pathology parameters. Decreased leukocytes (WBC) (down to 0.82×), lymphocytes (0.74×), monocytes (0.61×), eosinophils (0.50×), basophils (0.57×), and large unstained cells (0.73×), as well as increased neutrophils (1.2×) were noted in test article-treated females on Day 1 when compared to concurrent controls. These differences improved, but most were still present on Day 3 of the study. By Day 50, these values were similar to that of concurrent controls. Decreased activated partial thromboplastin time (0.76× and 0.80×) was noted in test article-treated females on Day 3 and Day 50 when compared to concurrent controls. Increased globulin (up to 1.59×) with associated decreased albumin to globulin ratio was noted in test article-treated males and females on Day 3 when compared to concurrent controls. These differences were not noted on Day 50.

Administration of SAB-176 by single intravenous infusion was well tolerated in rabbits at levels of 362.65 and 725.30 mg/kg/day. No target organs were observed. Based on these results, the no-observed-adverse-effect level (NOAEL) was considered to be 725.30 mg/kg/day.

### Example 7: Effect of SAB-176 in Humans

SAB-176 was evaluated in a Phase 1 study of 27 healthy volunteers at doses up to 50 mg/kg via intravenous injections. There were no reports of serious infusion-related reactions, allergic reactions, moderate to severe adverse events or any adverse events requiring discontinuation of therapy.

A Phase 2a, Randomized, Double-blind, Placebo-controlled study was also conducted to evaluate the safety and treatment efficacy of SAB-176 in an H1N1 challenge model in healthy adults. Sixty participants were randomized (1:1) to receive either SAB-176 (25 mg/kg dose) or placebo and were intranasally inoculated with influenza A/California/2009 H1N1 virus on Day 0 of the study. Participants were administered an IV infusion of SAB-176 or placebo on Day 1 and were held in quarantine until Day 8.

The primary efficacy analysis set was the Per Protocol (PP) analysis set that included 59 participants (29 in the SAB-176 group and 30 in the placebo group) and was defined based on the criteria of participants being challenged, dosed, and completing the quarantine up to Day 8. The efficacy analysis in this study was performed using the PP analysis set unless otherwise specified. The ITT-I (infected and intent to treat) analysis set included 27 participants (11 in the SAB-176 group and 16 in the placebo group) and was defined based on participants receiving IMP and were infected as per the definition of laboratory confirmed infection starting from Day 1 up to Day 8. The ITT-Is (infected and intent to treat sensitivity) analysis set included 25 participants (10 in the SAB-176 group and 15 in the placebo group); this sensitivity analysis set was defined based on participants receiving IMP and were infected as per the definition of laboratory confirmed infection starting from Day 2 up to Day 8. The ITT-Is analysis set was specifically chosen to assess the endpoints starting from Day 2 (24-hours post administration of SAB-176 or placebo) to consider the time taken for IV administered antibodies to get to the site of replication in the respiratory tract.

In this study, viral load was determined by qRT-PCR and viral cell culture assay to investigate a) infectivity status and rate, and b) viral dynamics (e.g., duration, peak, time to peak). In addition, symptom scores were gathered via a participant symptom diary card and questionnaires relating to participant cold perception, and nasal discharge collection from paper tissues was performed.

The one-sided statistical superiority approach with non-normal distribution of results in the SAB-176 cohort, as opposed to the placebo cohort, was pre-determined in the statistical analysis plan (SAP) and prior to reporting of previously blinded topline results. Statistical methods and analytical results are summarized in Table 12 and Table 13 below. This clinical study assumed that placebo would result in a normal distribution of Area-Under-Curve for viral load and symptomology metrics. This clinical study assumed that SAB-176 would significantly reduce or prevent viral load and symptomology metrics—it would not be a normal distribution that tended to zero across time. The tables and figures highlighted in this analysis demonstrate that the data is not normally distributed (by Student's T test analysis) and therefore the use of an a priori one-sided Wilcoxon rank sum test was appropriate. In Table 12 and Table 13, [1] indicates that the analysis is based on Student's t-distribution, [2] indicates mean SAB-176-mean Placebo, [3] indicates that the analysis is based on Satterthwaite test, assuming unequal variances; [4] indicates that the P-value of One-sided Wilcoxon rank sum test. In Table 12 and Table 13, % CV is derived for the AUC, based on the log 10 copies/mL.

TABLE 12
VL-AUC of Influenza A/California/2009 H1N1
as Determined by qRT-PCR

| Viral Load AUC | Treatment Group | |
|---|---|---|
| (Log10 copies*hour/mL) | SAB-176 | Placebo |
| n | 29 | 30 |
| Mean (SD) | 91.98 (166.034) | 273.05 (337.247) |
| % CV | 180.5 | 123.5 |
| SE | 30.832 | 61.573 |
| Median | 0.00 | 72.98 |
| Q1, Q3 | 0.00, 96.59 | 0.00, 589.96 |
| Min, Max | 0.0, 542.4 | 0.0, 963.5 |
| 90% CI of mean [1] | (39.53, 144.43) | (168.43, 377.67) |
| Difference in means [2] | −181.07 | |
| 90% CI for difference [2, 3] | (−296.85, −65.29) | |
| P-value [4] | 0.026 | |

TABLE 13
Area Under the Total Symptom Score-Time
Curve (TSS-AUC) Collected Daily in the Participant
Diary Card Per Protocol Analysis Set

| TSS-AUC (Total | | |
|---|---|---|
| Symptom Score | Treatment Group | |
| scale-points*hour) | SAB-176 | Placebo |
| n | 29 | 30 |
| Mean (SD) | 51.06 (74.559) | 166.52 (282.313) |
| % CV | 11.49 | 29.51 |
| SE | 146.0 | 169.5 |
| Median | 13.845 | 51.543 |
| Q1, Q3 | 19.51 | 33.99 |
| Min, Max | 0.0, 77.21 | 6.70, 137.12 |
| 90% CI of mean [1] | (27.50, 74.61) | (78.94, 254.09) |
| Difference in means [2] | −115.46 | |
| 90% CI for difference [2, 3] | (−205.77, −25.15) | |
| P-value [4] | 0.066 | |

The primary endpoint of the study was the reduction of the nasopharyngeal viral load of influenza A/California/2009 H1N1 virus in participants treated with SAB-176 (expressed as area under the curve, or AUC) compared to those receiving placebo over a period of 8 days, as measured by qRT-PCR. SAB-176 met the primary endpoint of significantly reducing H1N1 influenza viral load in the treated participants (p=0.026, one-sided for the PP analysis set). Similar results were observed for the infected analysis sets with the following one-sided p-values: p=0.006 (ITT-I) and p=0.003 (ITT-Is). Based on the variability observed in the placebo group, the power to detect a difference between the 2 treatment groups was calculated to be less than 80%.

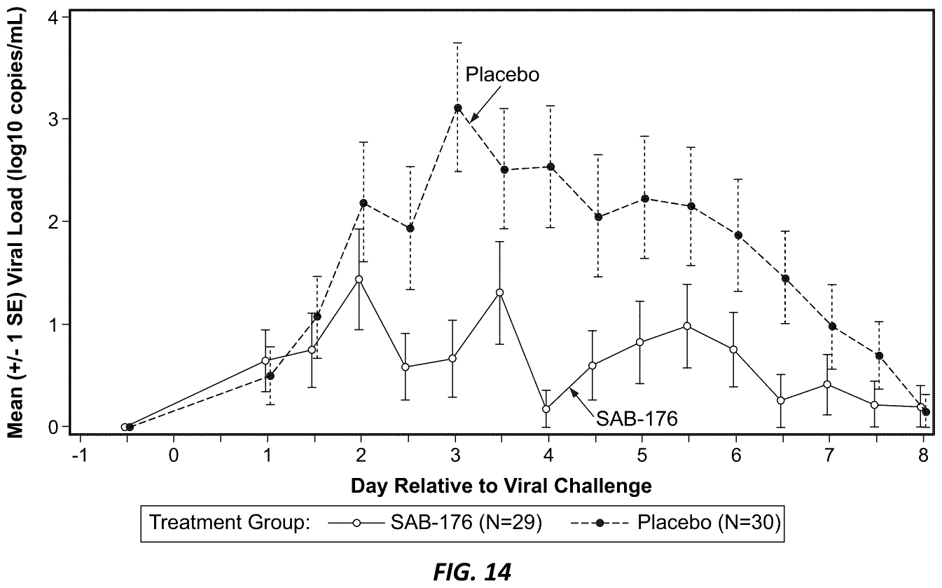

Viral AUC load over 8 days post pH1N1 challenge in per protocol treated placebo or SAB-176 participants demonstrates statistical significance that SAB-176 reduced viral load over placebo ((P-value of One-sided Wilcoxon rank sum test (0.026)) Table 12 and FIG. 14.

The findings were confirmed by the secondary endpoints, which showed significant reduction in H1N1 influenza VL-AUCs in the SAB-176 group compared to placebo (p=0.033 [PP], p=0.0019 [ITT-I], p=0.015 [ITT-Is]; one-sided) as measured by cell culture and further highlighted the anti-influenza efficacy of SAB-176. A lower mean qRT-PCR peak viral load of influenza A/California/2009 H1N1 virus was observed in the SAB-176 group compared to placebo; however, the difference was not statistically significant (p=0.057, one-sided); while when measured by cell culture a statistically significant lower (p=0.042, one-sided) peak viral load was observed in the SAB-176 group compared to placebo. Overall, the duration of viral shedding by both qRT-PCR and cell culture was shorter in the SAB-176 group compared to placebo.

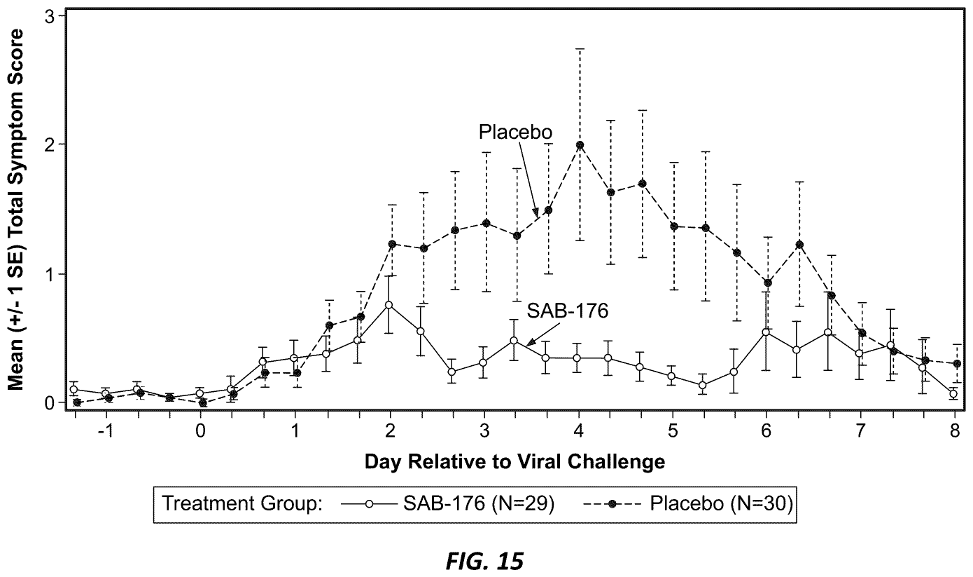

For all endpoints, similar results were observed for the ITT (where applicable), ITT-I, and ITT-Is analysis sets. Regarding symptom scores, lower mean TSS-AUC over time, lower mean peak TSS, and lower mean peak daily TSS were observed in the SAB-176 group compared to placebo; however, the differences were not statistically significant (p=0.066, p=0.065, and p=0.050, respectively, one-sided). Thus, symptomology AUC values over 8 days post pH1N1 challenge in per protocol treated placebo or SAB-176 participants demonstrated evidence that SAB-176 reduced clinically relevant symptoms ((P-value of One-sided Wilcoxon rank sum test (0.066)) (see Table 13 and FIG. 15). Similar results were observed for the ITT-I and ITT-Is analysis sets.

For incidence of grade 2 or higher symptoms no clear difference was shown between the treatment groups.

The incidence of RT-PCR-confirmed symptomatic influenza infection was statistically significant lower (p=0.038, one-sided) in the SAB-176 group compared to placebo for definition 1 defined as any 2 quantifiable qRT-PCR results over 4 consecutive scheduled timepoints, from morning of Day 2 up to Day 8 (discharge from quarantine) AND clinical symptoms (grade 2 or more symptoms) between Day 2 and quarantine discharge.

A lower incidence of symptomatic influenza infection was also observed in the SAB-176 group compared to placebo for definition 2 defined as any 2 quantifiable qRT-PCR results over 4 consecutive scheduled timepoints, from morning of Day 2 up to Day 8 and clinical symptoms (TSS of 5 or more) between Day 2 and quarantine discharge; however the difference was not statistically significant.

For culture lab-confirmed symptomatic influenza infection (both definitions), lower incidences were observed in the SAB-176 group compared to placebo; however, the differences were not statistically significant. The incidences in RT-PCR-confirmed and cell culture-confirmed influenza infections were lower in the SAB-176 group compared to placebo; however, the results did not show a statistically significant difference.

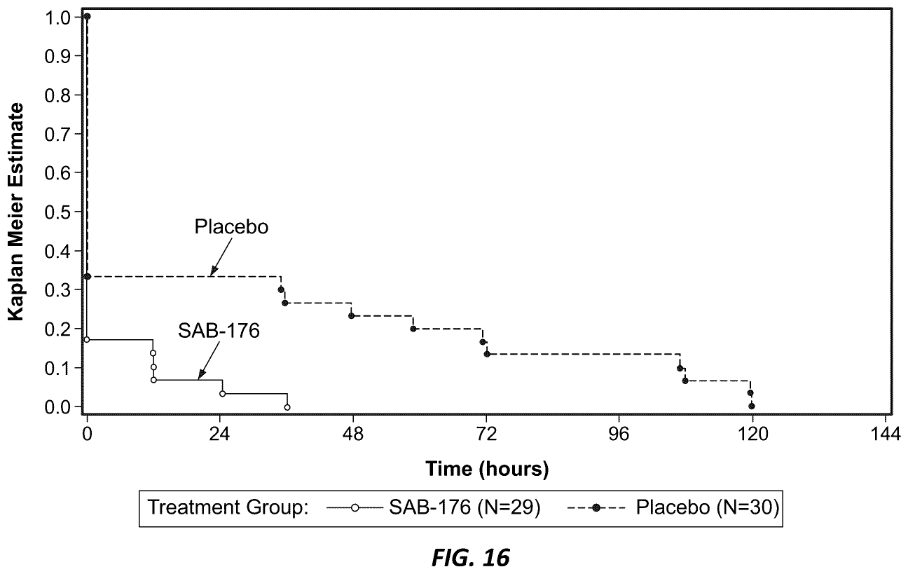

The duration of quantifiable cell culture was also measured. A Kaplan-Meier plot of viral load duration by nasal samples cell culture is presented in FIG. 16. In line with the qRT-PCR assay, for cell culture the upper quartile (Q3) of the duration to event (first confirmed unquantifiable assessment after which no further virus was detected) was 0 hours in the SAB-176 group and 47.7 hours in the placebo group for the PP analysis set. The viral load duration until all participants resolved measured by cell culture was 36 hours for SAB-176 and 120 hours for placebo. These results indicate that SAB-176 results in a shortened time of viral shedding, as measured by lack of culturable virus.

Regarding safety, overall, the challenge virus inoculation and subsequent single IV infusion of SAB-176 were safe and well tolerated. There were no serious adverse events (SAEs), no adverse events (AEs) leading to early withdrawal from the study, and none of the AEs were grade 3 or higher in severity. No SAB-176 related serious adverse events were observed, and most adverse events were mild to moderate in both the SAB-176 and placebo groups.

SAB-176 has met the study primary endpoint of reducing patient pH1N1 influenza viral load (p≤0.026) and demonstrated a trend towards reduction of clinical symptoms compared to the placebo. Further SAB-176 has been shown in this study to be safe and well tolerated. Based upon this efficacy and safety data SAB Biotherapeutics plans to further evaluate SAB-176 in advanced clinical trials.

### Example 8: Plasma Pooling Strategy

An effective therapeutic strategy for the treatment of influenza must broadly neutralize influenza viruses that are known to mutate. To accomplish this goal, Tc Bovines were repeatedly vaccinated with multiple years of seasonal influenza vaccine strains (as recommended by the World Health Organization [WHO] Global Influenza Programme).

Four lots of SAB-176 were tested for HAI titers and compared to three lots of human anti-flu IVIG that were produced in 2013, 2017 and 2018 from human Fresh Frozen Plasma units with high HAI titers to H1N1 and H3N2 influenza and utilized in a Phase 2 clinical study in hospitalized influenza patients (Clinicaltrials.gov Identifier: NCT02287467). SAB-176, human anti-flu IVIG and a negative control antibody (NC Ab) were all standardized to 5 mg/ml. The four lots of SAB-176 were tested against a panel of 14 seasonal/pandemic influenza viruses and compared with human anti-influenza IVIG. The sequences of the viruses strains discussed herein are publicly available, including for example at https://gisaid.org/register and https://www.ncbi.nlm.nih.gov/labs/virus/vssi/#/, which are incorporated herein by reference. The results are provided in Table 14. In Table 14, the number is the reciprocal of highest dilution factor that still resulted in hemagglutination inhibition. (The higher the number the more anti-flu activity is present in the sample). The virus strains that were not included as vaccine strains are indicated with an “*”.

TABLE 14
HAI titers

| | | | PD2102092QR | PD2203294QR | PD2204315QR | 23000994 | 23001746 |
|---|---|---|---|---|---|---|---|
| | PD1901102QR | PD2001010QR | 051221 | 113022 | 112822 | Anti-Flu | Anti-Flu |
| Virus Strain | DP Lot 1 | DP Lot 2 | DP Lot 4 | DP Lot 5 | DP Lot 6 | IVIG | IVIG |
| A/Michigan/45/2015 | 1280 | 1280 | 1810 | 1280 | 905 | 57 | 80 |
| A/Singapore/INFIMH- | 320 | 320 | 640 | 320 | 320 | 80 | 80 |
| 16-0019/2016 | | | | | | | |
| A/Brisbane/02/2018 | 1280 | 1280 | 1280 | 1280 | 1280 | 40 | 80 |
| A/Kansas/14/2017 | 40 | 40 | 320 | 320 | 320 | 14 | 14 |
| B/Colorado/6/2017 | 160 | 320 | 160 | 160 | 160 | 10 | 10 |
| A/Hawaii/70/2019 * | 1280 | 640 | 905 | 1810 | 1810 | 28 | 28 |
| A/Hong Kong/45/2019 | Does not react in HAI assays | | | | | | |
| A/Tasmania/503/2020 | 40 | 160 | 320 | 640 | 640 | 80 | 80 |
| (H3N2)* | | | | | | | |
| B/Washington/02/2019 | 160 | 160 | 160 | 320 | 320 | 10 | 10 |
| A/Wisconsin/588/2019* | 320 | 640 | 640 | 1280 | 1280 | 80 | 80 |
| A/Darwin/6/2021 * | 113 | 113 | 160 | 320 | 160 | 10 | 10 |
| B/Austria/ | 28 | 28 | 40 | 40 | 40 | 10 | 20 |
| 1359417/2021 * | | | | | | | |
| B/Phuket/3073/2013 | 160 | 160 | 160 | 80 | 160 | 5 | 10 |
| A/Sydney/5/2021* | 320 | 320 | 320 | 1280 | 1280 | 20 | 40 |
| A/California/4/2009 * | 1280 | 1280 | 1280 | 1280 | 640 | 80 | 80 |

Microneutralization (MN) Titers were also obtained as shown in Table 15. The number is the reciprocal of highest dilution factor that still resulted in inhibition of infection. The higher the number is indicative of greater anti-flu activity is present in the sample. The virus strains marked with “*” gray were not included as vaccine strains.

TABLE 15
Microneutralization (MN) Titers

| | | | PD2102092QR | PD2203294QR | PD2204315QR | 23000994 | 23001746 |
|---|---|---|---|---|---|---|---|
| | PD1901102QR | PD2001010QR | 051221 | 113022 | 112822 | Anti-Flu | Anti-Flu |
| Virus Strain | DP Lot 1 | DP Lot 2 | DP Lot 4 | DP Lot 5 | DP Lot 6 | IVIG | IVIG |
| A/Michigan/45/2015 | 10240 | 7241 | 10240 | 10240 | 10240 | 320 | 640 |
| (Oct. 4, 2019) | | | | | | | |
| A/Singapore/INFIMH- | 453 | 1810 | 1280 | 1280 | 1810 | 640 | 640 |
| 16-0019/2016 | | | | | | | |
| (Oct. 19, 2021) | | | | | | | |
| A/Brisbane/02/2018 | 1280 | 1280 | 1280 | 1810 | 1280 | 160 | 160 |
| (Oct. 4, 2019) | | | | | | | |
| A/kansas/14/2017 | 20 | 28 | 1280 | 905 | 1280 | 28 | 20 |
| (Oct. 4, 2019) | | | | | | | |
| B/Colorado/06/2017 | 640 | 2560 | 640 | 640 | 640 | 160 | 160 |
| (Oct. 19, 2021) | | | | | | | |
| A/Hawaii/70/2019 | 1280 | 905 | 1280 | 2560 | 2560 | 113 | 320 |
| (Dec. 10, 2020)* | | | | | | | |
| A/HongKong/45/2019 | 320 | 640 | 453 | 5120 | 3620 | 57 | 40 |
| (Jan. 14, 2019) | | | | | | | |
| A/Tasmania/503/2020 | 20 | 40 | 160 | 640 | 1280 | 57 | 40 |
| (Feb. 14, 2022)* | | | | | | | |
| B/Washington/02/2019 | 160 | 640 | 160 | 1280 | 1280 | 80 | 40 |
| (Dec. 23, 2019) | | | | | | | |
| A/Wisconsin/588/2019 | 640 | 905 | 1280 | 2560 | 2560 | 57 | 113 |
| (Dec. 17, 2020)* | | | | | | | |
| A/Darwin/6/2021 | 5 | 20 | 113 | 113 | 160 | 5 | 5 |
| (Feb. 3, 2022)* | | | | | | | |
| B/Austria/ | 80 | 160 | 160 | 320 | 640 | 40 | 40 |
| 1359417/2021 | | | | | | | |
| (Dec. 6, 2022)* | | | | | | | |
| B/Phuket/3073/2013 | 640 | 1280 | 1280 | 1280 | 905 | 80 | 160 |
| (Oct. 19, 2021) | | | | | | | |
| A/Sydney/5/2021 | 320 | 320 | 640 | 2560 | 1810 | 113 | 113 |
| (Dec. 6, 2022)* | | | | | | | |
| A/California/4/2009 | 2560 | 7241 | 2560 | 2560 | 2560 | 320 | 320 |
| (Oct. 18, 2021)* | | | | | | | |

The results for Lot PD1901102QR (Lot 1) and PD2102092QR (Lot 4) are presented in the table below (Table 16).

Lot PD1901102QR (Lot 1) was produced from a single season (2018/2019) quadrivalent antigen targeting Type A and B influenza strains and used to vaccinate Tc Bovine whereas Lot PD2102092QR (Lot 4) was produced using a seasonal quadrivalent antigen that spanned three seasons (2018/2019, 2019/2020, 2020/2021) targeting several seasonal variants to Type A and Type B influenza for the purpose of enhancing the cross-protective titers to current and future emerging mutations to both Type A and B influenza strains.

In Table 16, values indicate HAI titer. Numbers in parenthesis indicate the titer ratio of SAB-176 to hIVIG.

TABLE 16
HAI Titers

| | H1N1 | | | | | | |
|---|---|---|---|---|---|---|---|
| | A/California/ | | | | | H3N2 | |
| Sample | 4/2009 | | | A/Guangdong- | | A/Singapore/ | |
| Started at | (Pandemic | A/Michigan/ | A/Brisbane/ | maonan/ | A/Victoria/ | INIFMH- | A/Kansas/ |
| 5 mg/ml | Strain) | 45/2015 | 02/2018 | 2019 | 2570/19 | 16-0019/2016 | 14/2017 |
| SAB-176 | 512 | 512 | 512 | 512 | 256 | 512 | 64 |
| PD1901102QR | (8-16X) | (16X) | (16-32X) | (16-32X) | (16-32X) | (8-32X) | (2-16X) |
| (Lot 1) V3 | | | | | | | |
| SAB-176 | 512 | 512 | 512 | 512 | 256 | 512 | 256 |
| PD2102092QR | (8-16X) | (16X) | (16-32X) | (16-32X) | (16-32X) | (8-32X) | (8-64X) |
| (Lot 4) V3-V11 | | | | | | | |
| Human | 64 | 32 | 32 | 32 | 8 | 64 | 32 |
| Anti-Flu | | | | | | | |
| IVIG 2018 | | | | | | | |
| Human | 32 | 32 | 16 | 16 | 16 | 64 | 32 |
| Anti-Flu | | | | | | | |
| IVIG 2017 | | | | | | | |
| Human | 32 | 32 | 32 | 16 | 16 | 16 | 4 |
| Anti-Flu | | | | | | | |
| IVIG 2013 | | | | | | | |
| Negative | <1 | <1 | <1 | <1 | <1 | <1 | <1 |
| Control | | | | | | | |
| Antibody | | | | | | | |

| | H3N2 | | | | | | |
|---|---|---|---|---|---|---|---|
| Sample | A/Hong | | B-Victoria | | | B-Yamagata | |
| Started at | Kong/ | A/Cambodia/ | B/Maryland/ | B/Colorado/ | B/Washington/ | B/Phuket/ | B/California/ |
| 5 mg/ml | 45/2019 | e0826360 | 15/2016 | 06/2017 | 02/2019 | 3073/2013 | 12/2015 |
| SAB-176 | 64 | 128 | 256 | 256 | 64 | 128 | 128 |
| PD1901102QR | (4-8X) | (4-8X) | (16-32X) | (16-32X) | (8-16X) | (16X) | (16-32X) |
| (Lot 1) V3 | | | | | | | |
| SAB-176 | 256 | 256 | 256 | 256 | 128 | 128 | 128 |
| PD2102092QR | (16-32X) | (16-32X) | (16-32X) | (16-32X) | (16-32X) | (16X) | (16-32X) |
| (Lot 4) V3-V11 | | | | | | | |
| Human | 16 | 16 | 16 | 16 | 8 | 8 | 8 |
| Anti-Flu | | | | | | | |
| IVIG 2018 | | | | | | | |
| Human | 16 | 32 | 16 | 16 | 8 | 8 | 8 |
| Anti-Flu | | | | | | | |
| IVIG 2017 | | | | | | | |
| Human | 8 | 32 | 8 | 8 | 4 | 8 | 4 |
| Anti-Flu | | | | | | | |
| IVIG 2013 | | | | | | | |
| Negative | <1 | <1 | <1 | <1 | <1 | <1 | <1 |
| Control | | | | | | | |
| Antibody | | | | | | | |

SAB-176 was evaluated against strains used to vaccinate Tc Bovine to produce Lots PD1901102QR (Lot 1) and PD2102092QR (Lot 4) and compared to human anti-flu IVIG. SAB-176 showed HAI titers that were at 8-32 times higher than human anti-flu IVIG for H1N1, B-Victoria, and B-Yamagata influenza virus isolates shown in Table 16; numbers in parenthesis indicate the titer ratio of SAB-176 to human anti-Flu IVIG. The HAI titers for H3N2 influenza virus isolates were 2-64 times higher than human anti-flu IVIG.

SAB-176 also demonstrated HAI titers against non-target or non-vaccine (Tc Bovine not vaccinated against) strains of influenza and these titers were 2 to 32 times higher than corresponding titers in hIVIG. These higher HAI titers indicate SAB-176 may more effectively neutralize a variety of influenza strains compared to hIVIG.

Based on historical data from SAB-176 against vaccine strains, an initial plasma pooling strategy was developed. The two lots of SAB-176, described above, include plasma that was obtained after the fifth vaccination (V5) or later timepoint. Additionally, these lots were produced from a mixture of plasma collected from a production herd of Tc Bovine that received the seasonal quadrivalent antigen each year consecutively spanning up to four seasonal periods (see Table 17). Hyperimmune plasma ≥V5 for each influenza season was collected and the percentage of plasma used to produce the respective lot from each representative season is indicated in Table 17.

TABLE 17
HEMAGGLUTINATION INHIBITION (HAI) TITERS FOR
SAB-176 DRUG PRODUCT (DP)/DRUG SUBSTANCE (DS)

| | Influenza | Percent | | | | |
|---|---|---|---|---|---|---|
| SAB-176 Lots | Season(s) | Plasma | H1N1 | H3N2 | B-Victoria | B-Yamagata |
| PD1901102QR | 2018-19 | 100% | 7,680 | 7,680 | 3840 | 1920 |
| (Lot 1)(DP) | | | A/Michigan/ | A/Singapore/ | B/Maryland/ | B/Phuket/ |
| | | | 45/2015 | INFIMH- | 15/2016 | 3073/2013 |
| | | | | 16-0019/2016 | | |
| PD2001010QR | 2018-19 | 100% | 14336 | 7168 | 3584 | 3584 |
| (Lot 2)(DP) | | | A/Michigan/ | A/Singapore/ | B/Maryland/ | B/Phuket/ |
| | | | 45/2015 | INFIMH- | 15/2016 | 3073/2013 |
| | | | | 16-0019/2016 | | |
| PD2102092QR | 2018-19 | 73% | 7680 | 1920 | 960 | 3840 |
| (Lot 4)(DP) | 2019-20 | 24% | | | | |
| | 2020-21 | 3% | A/Guangdong- | A/Hong Kong/ | B/Washington/ | B/Phuket/ |
| | | | maonan/2019 | 45/2019 | 02/2019 | 3073/2013 |
| PD2203294QR | 2018-19 | 10% | 7680 | 3840 | 3840 | 1920 |
| (Lot 5)(DS) | 2019-20 | 13% | | | | |
| | 2020-21 | 27% | | | | |
| | 2021-22 | 53% | A/Victoria/ | A/Cambodia/ | B/Washington/ | B/Phuket/ |
| | | | 2570/19 | e0826360 | 02/2019 | 3073/2013 |
| PD2204315QR | 2018-19 | 10% | 7680 | 7680 | 3840 | 1920 |
| (Lot 6)(DS) | 2019-20 | 13% | | | | |
| | 2020-21 | 26% | | | | |
| | 2021-22 | 54% | A/Victoria/ | A/Cambodia/ | B/Washington/ | B/Phuket/ |
| | | | 2570/19 | e0826360 | 02/201 | 3073/2013 |

All quadrivalent vaccines given to Tc Bovine each season is matched to the recommended Northern Hemisphere strains for that season. The results in Table 17 indicate high titers are maintained with the pooling strategy to the latest season used to produce the designated lot.

Plasma data and clinical study data will be used for continued plasma pooling evolution of SAB-176.

### Example 9: Generation of H7N9 Specific Human Polyclonal Antibodies from a Transchromosomic Goat (Caprine) System

To demonstrate the applicability of Tc bovine platform to other ungulate systems, the transgenic goat (also herein “Tc Capra”) system was established.

To generate the Tc Capra systems, the animals were engineered to include an isKcHACΔ, a human artificial chromosome (HAC) that comprise the entire human Ig genetic repertoire in the germline configuration in which the regulatory genomic sequences involved in pre-B cell receptor (preBCR) and BCR signaling during B cell development and those mediating human Ig class switch recombination are replaced with the respective genomic sequences from an ungulate (bovine).

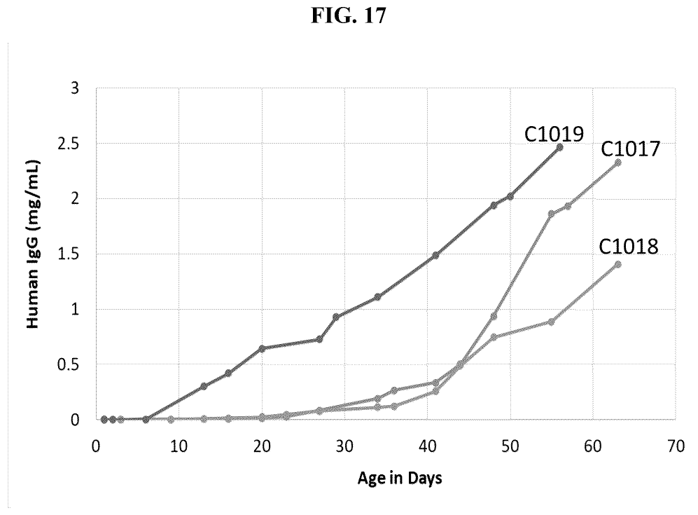

To evaluate human IgG levels in the Tc Capra, human IgG ELISA was performed on the Tc Capra serum samples. At day 60, about 1-2.5 mg/ml of human IgG was detected int the Tc Capra sera (see FIG. 17).

To investigate whether the Tc Capra can mount hIgG-mediated humoral immune response to antigens, the Tc Capras at the age of 5 months were immunized with 0.2 mg to 0.5 mg of recombinant H7N9 HAG protein from the first vaccination to the fifth vaccination. At day 10 and 14 post the third, fourth and fifth vaccination, plasmas were collected from the immunized goats from which hIgG was purified. In vitro virus neutralization efficacy against influenza A (H7N9) viruses was performed using purified fully human IgG from the pooled the Tc Capra plasma. The results were compared to SAB-127 produced from Tc bovine immunized with inactivated H7N9 virus (A/Anhui/2013) and a negative control antibody (NC Ab). The results are shown in Table 18.

TABLE 18
In vitro virus neutralization efficacy against influenza A (H7N9)

| | Neutralizing Concentration (μg/mL) | | | |
|---|---|---|---|---|
| | | | A/Hong | |
| | | | Kong/ | |
| | A/Anhui/ | A/Hong Kong/ | 125/2017 | A/Taiwan/ |
| Antibody | 1/2013 | 61/2016 | 1/2016 | 1/2017 |
| Tc Goat | 2.4 | 4.7 | 4.7 | 4.7 |
| H7N9 | | | | |
| SAB-127 | 4.7 | 9.4 | 4.7 | 9.4 |
| NC Ab | >300 | >300 | >300 | >300 |

As shown in Table 18, Tc Capra-derived anti-H7N9 human IgG showed neutralizing capacity to various H7N9 strains. As shown in Table 18, both TC bovine and goats immunized with H7N9 antigen derived an immune response with fully human polyclonal antibodies. This establishes the ability of the human artificial chromosome to be used in other ungulate species.

Tc Capra-derived anti-H7N9 human IgG were evaluated for protection against an influenza A/Anhui/1/2013 (H7N9) virus infection in BALB/c mice. Groups of mice were treated by intraperitoneal (i.p.) injection on a single occasion with 12.5, 25, 50, and 100 mg/kg doses of Tc goat-derived anti-H7N9 human IgG, or a 50 mg/kg dose of SAB-127, a Tc bovine anti-H7N9 antibody used as a positive control. Placebo, 100 mg/kg control antibody, was administered in a similar manner to the Tc Capra-derived human polyclonal antibodies. In addition, 40 mg/kg oseltamivir was administered twice daily for 5 days by oral gavage (p.o.) and served as positive control for the virus challenge dose. Three non-treated control mice were maintained for weight comparison. For influenza virus challenge, mice were anesthetized by i.p. injection of ketamine/xylazine (50 mg/kg//5 mg/kg) prior to challenge by the intranasal route with 5×10³ CCID₅₀ (1×LD₉₀) of influenza A/Anhui/1/2013 (H7N9) virus per mouse in a 90 μl volume per mouse. All mice were administered virus challenge on study day 21. Mice were weighed prior to treatment and then daily thereafter to assess the effects of treatment on ameliorating weight loss due to virus infection. All mice were observed for morbidity and mortality through day 21.

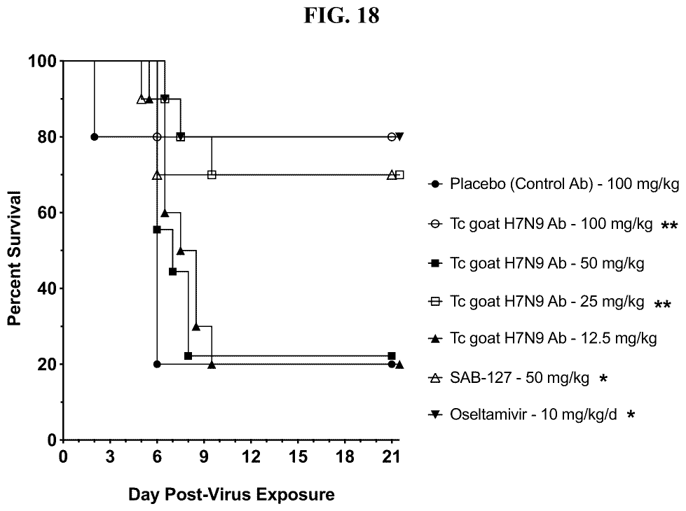

FIG. 18 shows survival results following challenge infection and a single treatment with the Tc Capra-derived anti-H7N9 human IgG. Both 25 and 100 mg/kg doses of antibody provided 70 and 80% protection, respectively. The 50 mg/kg dose of the goat-derived antibody did not provide protection. In addition, the 50 mg/kg dose of SAB-127, a Tc bovine anti-H7N9 antibody used as a positive control provided 70% protection.

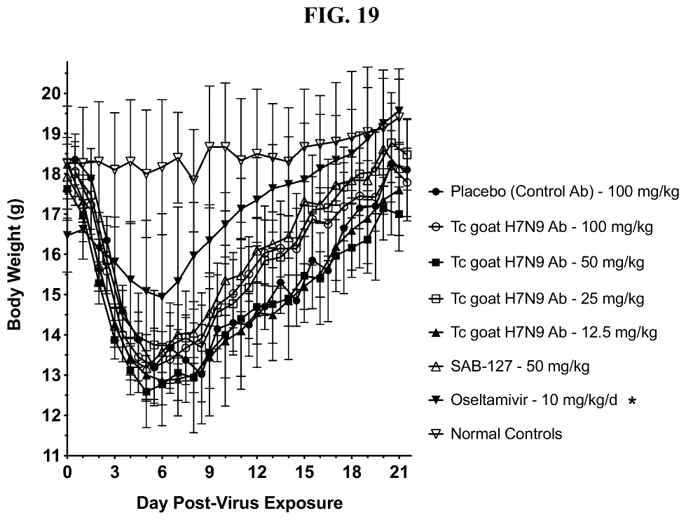

Mean body weights following treatment with goat-derived human IgG, after virus challenge are shown in FIG. 19. None of the groups treated with the goat-derived antibody showed protection from weight loss. However, the oseltamivir treated group, used as the control for the virus challenge dose did show protection from challenge, indicating that the challenge dose was as expected.

Taken together, these data demonstrate that the functionality of the HAC systems in a second ungulate species (specifically a ruminant ungulate).

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. An ungulate-derived polyclonal human immunoglobulin composition, comprising a population of ungulate-derived polyclonal human immunoglobulins,
wherein the population of ungulate-derived polyclonal human immunoglobulins specifically binds an influenza hemagglutinin (HA) protein of two or more strains of influenza.

2. The composition of claim 1, wherein the influenza HA protein of the two or more strains of influenza is an HA protein of Influenza A and an HA protein of Influenza B.

3. The composition of claim 1, wherein the influenza HA proteins of the two or more strains of influenza comprises a full-length influenza HA1 protein and a full-length influenza HA2 protein.

4. The composition of claim 1, wherein the population of ungulate-derived polyclonal human immunoglobulins has a neutralizing concentration of at least 0.01 μg/ml, at least 0.1 μg/ml, or at least 1.0 μg/ml.

5. The composition of claim 1, wherein the population of ungulate-derived polyclonal human immunoglobulins has an avidity for influenza HA protein of at least 0.1 1/sec, at least 0.01 1/sec, at least 0.001 1/sec at least 0.0001 1/sec, or at least 0.00001 1/sec.

6. The composition of claim 1, comprising glycans covalently linked to the population of ungulate-derived polyclonal human immunoglobulins, wherein the glycans are at least about 70% N-Glycolylneuraminic acid (NGNA) glycans.

7. The composition of claim 6, comprising glycans covalently linked to the population of ungulate-derived polyclonal human immunoglobulins, wherein the population of human immunoglobulins comprise less than about 50% N-Acetylneuraminic acid (NANA) glycans.

8. The composition of claim 1, wherein the population of ungulate-derived polyclonal human immunoglobulins comprise less than 5% chimeric IgG immunoglobulins.

9. The composition of claim 1, wherein the population of ungulate-derived polyclonal human immunoglobulins comprise less than 5% chimeric IgM immunoglobulins.

10. The composition of claim 1, wherein the population of ungulate-derived polyclonal human immunoglobulins comprise at least about 70% of IgG1.

11. The composition of claim 1, wherein the population of ungulate-derived polyclonal human immunoglobulins comprise less than about 30% IgG2.

12. The composition of claim 1, wherein the population of ungulate-derived polyclonal immunoglobulins comprise less than 4% of one or more of IgG3 and IgG4.

13. A method of making an ungulate-derived polyclonal human immunoglobulin composition specific for influenza hemagglutinin (HA) protein, comprising administering an effective amount of an influenza HA protein from at least two or more strains of influenza, or a polynucleotide encoding an influenza HA protein from at least two or more strains of influenza, to one or more transgenic ungulates, wherein the one or more transgenic ungulates comprise a genome comprising a human immunoglobulin locus or an artificial chromosome comprising a human immunoglobulin locus, and
purifying a population of ungulate-derived polyclonal human immunoglobulins from serum or plasma of the one or more transgenic ungulates and
combining the ungulate-derived polyclonal human immunoglobulins purified from the one or more transgenic ungulates;
wherein the ungulate-derived polyclonal human immunoglobulin composition is made.

14. The method of claim 13, comprising administering the influenza HA protein 3, 4, 5, or more times.

15. The method of claim 13, wherein the influenza HA protein comprises one or more of a full-length influenza HA1 protein and a full-length influenza HA2 protein.

16. The method of claim 13, wherein the population of ungulate-derived polyclonal human immunoglobulins has a neutralizing concentration of at least 0.01 μg/ml, at least 0.1 μg/ml, or at least 1.0 μg/ml.

17. The method of claim 13, wherein the population of ungulate-derived polyclonal human immunoglobulins has an avidity for influenza HA protein of at least 0.1 1/sec, at least 0.01 1/sec, at least 0.001 1/sec at least 0.0001 1/sec, or at least 0.00001 1/sec.

18. The method of claim 13, wherein comprising administering the transgenic ungulate with about 0.1 to 10 mg of the influenza HA protein.

19. An ungulate-derived polyclonal human immunoglobulin composition specific for influenza HA protein, prepared by the process of administering an effective amount of an influenza HA protein from at least two or more strains of influenza, or a polynucleotide encoding an influenza HA protein from at least two or more strains of influenza to one or more transgenic ungulates,
wherein the one or more transgenic ungulates comprise a genome comprising a human immunoglobulin locus or an artificial chromosome comprising a human immunoglobulin locus, and
purifying a population of ungulate-derived polyclonal human immunoglobulins from serum or plasma of the one or more transgenic ungulates;
combining the ungulate-derived polyclonal human immunoglobulins purified from the one or more transgenic ungulates,
wherein the ungulate-derived polyclonal human immunoglobulin composition is made.

20. A pharmaceutical composition, comprising the composition of claim 1 and optionally one or more pharmaceutically acceptable excipients.

21. A method of treating infection with influenza virus in a subject in need thereof, comprising administering an effective amount of the composition of claim 1 to the subject.
